SEQUENCE LISTING (36 sequences)

SEQ ID NO: 1 - DNA, 483 bp, synthetic construct
  source 1..483: mol_type = other DNA
agccctggccagggcacccaatctgaaaattcctgcacccacttccccggcaacctgcctaatatgctgagagatctgcgcgacgcattcagcagagtgaagacattcttccaaatgaaggaccagctggacaacctgctgcttaaagagagcctgctggaagattttaagggatatctgggatgtcaggctctgtctgagatgatccagttctacctggaagaggtaatgcctcaggccgagaaccaggaccccgatattaaggcccatgtgaactccctgggcgagaatctgaagaccctgcggctgagactgcggagatgccacagattcctgccatgtgaaaacaaaagcaaggccgtggaacaggtcaagaacgcatttaacaagctgcaggagaagggcatctacaaggctatgagcgagttcgacatcttcatcaactacatcgaggcctacatgacaatgaaaatcagaaactga

SEQ ID NO: 2 - DNA, 537 bp, synthetic construct
  source 1..537: mol_type = other DNA
atgcacagcagcgccctgctgtgctgcctggtgctgctcaccggagtgcgggccagccctggccagggcacccaatctgaaaattcctgcacccacttccccggcaacctgcctaatatgctgagagatctgcgcgacgcattcagcagagtgaagacattcttccaaatgaaggaccagctggacaacctgctgcttaaagagagcctgctggaagattttaagggatatctgggatgtcaggctctgtctgagatgatccagttctacctggaagaggtaatgcctcaggccgagaaccaggaccccgatattaaggcccatgtgaactccctgggcgagaatctgaagaccctgcggctgagactgcggagatgccacagattcctgccatgtgaaaacaaaagcaaggccgtggaacaggtcaagaacgcatttaacaagctgcaggagaagggcatctacaaggctatgagcgagttcgacatcttcatcaactacatcgaggcctacatgacaatgaaaatcagaaactga

SEQ ID NO: 3 - DNA, 483 bp, Homo sapiens
  source 1..483: mol_type = genomic DNA
agcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 4 - DNA, 537 bp, Homo sapiens
  source 1..537: mol_type = genomic DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 5 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 6 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 7 - DNA, 459 bp, synthetic construct
  source 1..459: mol_type = other DNA
agacctagcggaagaaagagcagcaagatgcaggcctttagaatctgggatgttaatcagaaaaccttctacctgcggaacaaccagctggtcgccggctacctgcagggccccaacgtgaatctggaagagaagatcgacgtggtgccaatcgagccccacgccctgttcctgggcatccatggcggaaaaatgtgcctgagctgcgtgaagtccggcgacgaaaccagactgcaactcgaggccgtgaacattaccgacctgtctgagaaccggaagcaagacaagcgcttcgcattcatcagaagcgacagcggccctaccacaagcttcgagtctgccgcttgtcctggctggttcctgtgcacagccatggaagctgatcagcctgtgtccctgaccaacatgcctgatgaaggcgtgatggtgaccaagttctacttccaggaggacgagtga

SEQ ID NO: 8 - DNA, 534 bp, synthetic construct
  source 1..534: mol_type = other DNA
atggaaatctgcaggggcctgagatctcacctgatcaccctgctgctttttctgttccacagcgagacaatctgtagacctagcggaagaaagagcagcaagatgcaggcctttagaatctgggatgttaatcagaaaaccttctacctgcggaacaaccagctggtcgccggctacctgcagggccccaacgtgaatctggaagagaagatcgacgtggtgccaatcgagccccacgccctgttcctgggcatccatggcggaaaaatgtgcctgagctgcgtgaagtccggcgacgaaaccagactgcaactcgaggccgtgaacattaccgacctgtctgagaaccggaagcaagacaagcgcttcgcattcatcagaagcgacagcggccctaccacaagcttcgagtctgccgcttgtcctggctggttcctgtgcacagccatggaagctgatcagcctgtgtccctgaccaacatgcctgatgaaggcgtgatggtgaccaagttctacttccaggaggacgagtga

SEQ ID NO: 9 - DNA, 459 bp, Homo sapiens
  source 1..459: mol_type = genomic DNA
cgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtag

SEQ ID NO: 10 - DNA, 534 bp, Homo sapiens
  source 1..534: mol_type = genomic DNA
atggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtag

SEQ ID NO: 11 - protein, 152 aa, Homo sapiens
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 12 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 13 - DNA, 1482 bp, synthetic construct
  source 1..1482: mol_type = other DNA
tgttgcggaccacctccacttagaccaccattgccagcagctgcagctgccgcagccggtggacagctgctgggagatggcggctctcctggcaggaccgagcagcctcctccaagccctcaatcttcgagcggcttcctgtaccggagactcaaaacccaggagaagcgggaaatgcagaaagagatcctgagcgtgctgggcctgcctcaccggcccagacctctgcacggcctgcagcaaccgcagccaccagccctgcggcaacaggaagagcagcaacagcaacagcaactgcctagaggcgagcctcctcctggcagactgaaatctgctcctctgttcatgctggatctgtataacgccctgagcgccgataacgacgaggacggcgcctctgagggagaaagacaacagagctggcctcatgaggcagccagcagctcccagagaagacagcctcctccaggagctgcacaccctctgaatagaaagagcctgctggctcctggttccggcagcggcggagcctcccccctgaccagcgctcaggacagcgcttttctgaacgacgccgacatggttatgagcttcgtgaacctggtggaatacgataaggaattcagccctagacagcggcaccacaaggagttcaagttcaacctgtctcagatccccgagggcgaggtggtgacagccgccgagttcagaatctataaggactgcgtgatgggcagcttcaaaaaccagaccttcctgatctccatctaccaggtgctgcaagaacaccagcatcgggatagcgacctgttcctgctggacaccagagtggtgtgggccagcgaagaaggctggctggaatttgacatcaccgccacaagcaacctgtgggtcgtcacacctcagcacaacatgggactgcagctgagcgtggttacccgcgacggcgtgcacgtgcaccccagagccgcaggcctggtgggccgggacggaccttacgacaagcagcctttcatggtggccttcttcaaggtgtccgaggtgcacgtgcggaccacccggtctgcctcatctagacggagacagcagagcagaaacagaagcacacagtctcaggatgtggccagggtgagcagcgcctctgactacaacagcagcgaactgaagacagcatgccggaagcacgagctgtacgtgtccttccaggatctgggctggcaggactggatcatcgcccccaagggctacgccgctaactactgcgacggagagtgcagctttcctctcaacgcccacatgaacgccaccaaccacgccattgtgcagaccctggtgcacctgatgaatcccgagtacgtgccaaagccctgttgcgcccctacaaagctgaatgccatcagcgtgctgtacttcgacgataattctaatgtgatcctgaagaagtacaggaacatggtggtcagagcttgtggttgtcactga

SEQ ID NO: 14 - DNA, 1542 bp, synthetic construct
  source 1..1542: mol_type = other DNA
atgcctggcctgggcagaagagcccagtggctgtgctggtggtggggactgctctgtagctgttgcggaccacctccacttagaccaccattgccagcagctgcagctgccgcagccggtggacagctgctgggagatggcggctctcctggcaggaccgagcagcctcctccaagccctcaatcttcgagcggcttcctgtaccggagactcaaaacccaggagaagcgggaaatgcagaaagagatcctgagcgtgctgggcctgcctcaccggcccagacctctgcacggcctgcagcaaccgcagccaccagccctgcggcaacaggaagagcagcaacagcaacagcaactgcctagaggcgagcctcctcctggcagactgaaatctgctcctctgttcatgctggatctgtataacgccctgagcgccgataacgacgaggacggcgcctctgagggagaaagacaacagagctggcctcatgaggcagccagcagctcccagagaagacagcctcctccaggagctgcacaccctctgaatagaaagagcctgctggctcctggttccggcagcggcggagcctcccccctgaccagcgctcaggacagcgcttttctgaacgacgccgacatggttatgagcttcgtgaacctggtggaatacgataaggaattcagccctagacagcggcaccacaaggagttcaagttcaacctgtctcagatccccgagggcgaggtggtgacagccgccgagttcagaatctataaggactgcgtgatgggcagcttcaaaaaccagaccttcctgatctccatctaccaggtgctgcaagaacaccagcatcgggatagcgacctgttcctgctggacaccagagtggtgtgggccagcgaagaaggctggctggaatttgacatcaccgccacaagcaacctgtgggtcgtcacacctcagcacaacatgggactgcagctgagcgtggttacccgcgacggcgtgcacgtgcaccccagagccgcaggcctggtgggccgggacggaccttacgacaagcagcctttcatggtggccttcttcaaggtgtccgaggtgcacgtgcggaccacccggtctgcctcatctagacggagacagcagagcagaaacagaagcacacagtctcaggatgtggccagggtgagcagcgcctctgactacaacagcagcgaactgaagacagcatgccggaagcacgagctgtacgtgtccttccaggatctgggctggcaggactggatcatcgcccccaagggctacgccgctaactactgcgacggagagtgcagctttcctctcaacgcccacatgaacgccaccaaccacgccattgtgcagaccctggtgcacctgatgaatcccgagtacgtgccaaagccctgttgcgcccctacaaagctgaatgccatcagcgtgctgtacttcgacgataattctaatgtgatcctgaagaagtacaggaacatggtggtcagagcttgtggttgtcactga

SEQ ID NO: 15 - DNA, 1482 bp, Homo sapiens
  source 1..1482: mol_type = genomic DNA
tgctgcgggcccccgccgctgcggccgcccttgcccgctgccgcggccgccgccgccggggggcagctgctgggggacggcgggagccccggccgcacggagcagccgccgccgtcgccgcagtcctcctcgggcttcctgtaccggcggctcaagacgcaggagaagcgggagatgcagaaggagatcttgtcggtgctggggctcccgcaccggccccggcccctgcacggcctccaacagccgcagcccccggcgctccggcagcaggaggagcagcagcagcagcagcagctgcctcgcggagagccccctcccgggcgactgaagtccgcgcccctcttcatgctggatctgtacaacgccctgtccgccgacaacgacgaggacggggcgtcggagggggagaggcagcagtcctggccccacgaagcagccagctcgtcccagcgtcggcagccgcccccgggcgccgcgcacccgctcaaccgcaagagccttctggcccccggatctggcagcggcggcgcgtccccactgaccagcgcgcaggacagcgccttcctcaacgacgcggacatggtcatgagctttgtgaacctggtggagtacgacaaggagttctcccctcgtcagcgacaccacaaagagttcaagttcaacttatcccagattcctgagggtgaggtggtgacggctgcagaattccgcatctacaaggactgtgttatggggagttttaaaaaccaaacttttcttatcagcatttatcaagtcttacaggagcatcagcacagagactctgacctgtttttgttggacacccgtgtagtatgggcctcagaagaaggctggctggaatttgacatcacggccactagcaatctgtgggttgtgactccacagcataacatggggcttcagctgagcgtggtgacaagggatggagtccacgtccacccccgagccgcaggcctggtgggcagagacggcccttacgacaagcagcccttcatggtggctttcttcaaagtgagtgaggtgcacgtgcgcaccaccaggtcagcctccagccggcgccgacaacagagtcgtaatcgctctacccagtcccaggacgtggcgcgggtctccagtgcttcagattacaacagcagtgaattgaaaacagcctgcaggaagcatgagctgtatgtgagtttccaagacctgggatggcaggactggatcattgcacccaagggctatgctgccaattactgtgatggagaatgctccttcccactcaacgcacacatgaatgcaaccaaccacgcgattgtgcagaccttggttcaccttatgaaccccgagtatgtccccaaaccgtgctgtgcgccaactaagctaaatgccatctcggttctttactttgatgacaactccaatgtcattctgaaaaaatacaggaatatggttgtaagagcttgtggatgccactaa

SEQ ID NO: 16 - DNA, 1542 bp, Homo sapiens
  source 1..1542: mol_type = genomic DNA
atgccggggctggggcggagggcgcagtggctgtgctggtggtgggggctgctgtgcagctgctgcgggcccccgccgctgcggccgcccttgcccgctgccgcggccgccgccgccggggggcagctgctgggggacggcgggagccccggccgcacggagcagccgccgccgtcgccgcagtcctcctcgggcttcctgtaccggcggctcaagacgcaggagaagcgggagatgcagaaggagatcttgtcggtgctggggctcccgcaccggccccggcccctgcacggcctccaacagccgcagcccccggcgctccggcagcaggaggagcagcagcagcagcagcagctgcctcgcggagagccccctcccgggcgactgaagtccgcgcccctcttcatgctggatctgtacaacgccctgtccgccgacaacgacgaggacggggcgtcggagggggagaggcagcagtcctggccccacgaagcagccagctcgtcccagcgtcggcagccgcccccgggcgccgcgcacccgctcaaccgcaagagccttctggcccccggatctggcagcggcggcgcgtccccactgaccagcgcgcaggacagcgccttcctcaacgacgcggacatggtcatgagctttgtgaacctggtggagtacgacaaggagttctcccctcgtcagcgacaccacaaagagttcaagttcaacttatcccagattcctgagggtgaggtggtgacggctgcagaattccgcatctacaaggactgtgttatggggagttttaaaaaccaaacttttcttatcagcatttatcaagtcttacaggagcatcagcacagagactctgacctgtttttgttggacacccgtgtagtatgggcctcagaagaaggctggctggaatttgacatcacggccactagcaatctgtgggttgtgactccacagcataacatggggcttcagctgagcgtggtgacaagggatggagtccacgtccacccccgagccgcaggcctggtgggcagagacggcccttacgacaagcagcccttcatggtggctttcttcaaagtgagtgaggtgcacgtgcgcaccaccaggtcagcctccagccggcgccgacaacagagtcgtaatcgctctacccagtcccaggacgtggcgcgggtctccagtgcttcagattacaacagcagtgaattgaaaacagcctgcaggaagcatgagctgtatgtgagtttccaagacctgggatggcaggactggatcattgcacccaagggctatgctgccaattactgtgatggagaatgctccttcccactcaacgcacacatgaatgcaaccaaccacgcgattgtgcagaccttggttcaccttatgaaccccgagtatgtccccaaaccgtgctgtgcgccaactaagctaaatgccatctcggttctttactttgatgacaactccaatgtcattctgaaaaaatacaggaatatggttgtaagagcttgtggatgccactaa

SEQ ID NO: 17 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 18 - protein, 493 aa, Homo sapiens
CCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 19 - DNA, 399 bp, synthetic construct
  source 1..399: mol_type = other DNA
gtgaagatgcacaccatgagcagcagccacctcttttacctggccctgtgcctgctgaccttcaccagcagcgctacagccggacctgagaccctgtgtggcgccgagctggtcgatgccctgcagttcgtgtgcggagacagaggcttctatttcaacaagcctacaggctacggctctagctccagaagagccccacagaccggcatcgtggacgagtgctgttttagaagctgtgatctgagaaggctggaaatgtactgcgcccctctgaaacccgctaagtcagccagatccgtgcgggcccagcggcacacagacatgcctaagacccaaaaggaagtgcatctgaagaatgcctctagaggatctgctggaaacaagaactaccggatgtga

SEQ ID NO: 20 - DNA, 462 bp, synthetic construct
  source 1..462: mol_type = other DNA
atgggcaagatcagcagcctgcccacacagctgttcaagtgctgcttctgcgacttcctgaaggtgaagatgcacaccatgagcagcagccacctcttttacctggccctgtgcctgctgaccttcaccagcagcgctacagccggacctgagaccctgtgtggcgccgagctggtcgatgccctgcagttcgtgtgcggagacagaggcttctatttcaacaagcctacaggctacggctctagctccagaagagccccacagaccggcatcgtggacgagtgctgttttagaagctgtgatctgagaaggctggaaatgtactgcgcccctctgaaacccgctaagtcagccagatccgtgcgggcccagcggcacacagacatgcctaagacccaaaaggaagtgcatctgaagaatgcctctagaggatctgctggaaacaagaactaccggatgtga

SEQ ID NO: 21 - DNA, 399 bp, Homo sapiens
  source 1..399: mol_type = genomic DNA
gtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtag

SEQ ID NO: 22 - DNA, 462 bp, Homo sapiens
  source 1..462: mol_type = genomic DNA
atgggaaaaatcagcagtcttccaacccaattatttaagtgctgcttttgtgatttcttgaaggtgaagatgcacaccatgtcctcctcgcatctcttctacctggcgctgtgcctgctcaccttcaccagctctgccacggctggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgtag

SEQ ID NO: 23 - protein, 132 aa, Homo sapiens
VKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 24 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 25 - DNA, 1170 bp, synthetic construct
  source 1..1170: mol_type = other DNA
ctgtctacatgcaccaccctggatttcggccacatcaagaaaaaacgggtcgaggccatcagaggtcagatcctgagcaagctgaggctgaccagccctccagagcctaccgtgatgacccacgtgccttaccaggtgctggccctgtataattctacaagagagctgctggaagagatgcacggagaaagagaggaaggctgtacccaggagaataccgagagcgagtactacgccaaggaaatccacaagttcgacatgatccagggcctggctgaacataacgagctggccgtgtgccctaagggcatcaccagcaaggtgttcagattcaacgtgtccagcgtggaaaagaacagaacaaacctgtttcgggccgagttccgggtgctgagagtgcccaatccttcttccaagcggaacgagcagagaatcgaactgttccaaatcctgagacccgatgagcacatcgctaaacagagatacatcggcggcaagaacctgcccacacggggcaccgccgagtggctgagctttgatgtgaccgacaccgtgagagagtggctgctgcggcgggagagcaacctgggcctcgaaatcagcatccactgcccttgccacacattccagcctaacggcgacatcctggaaaacatccacgaggtgatggaaattaagtttaagggcgtggacaacgaggacgaccacggccgcggcgatctgggcagactgaagaagcagaaggaccaccacaacccccacctgattctcatgatgatccctccacacagactggataaccctggacagggcggacaaagaaagaagagagccctggacaccaactactgcttccggaacctggaagagaactgctgcgtgcggccactgtacatcgacttcagacaggatctgggatggaagtgggtgcacgagcccaaaggctactacgctaacttctgctcaggcccttgcccttacctgagaagcgccgacacaacccacagcacagtgctgggactgtacaacaccctgaacccagaggccagcgccagcccctgttgtgtgccacaggacctcgagcctctgacaatcctgtactatgtgggcagaacccctaaggtcgaacagctgtctaatatggtggtgaagtcttgtaaatgcagctga

SEQ ID NO: 26 - DNA, 1239 bp, synthetic construct
  source 1..1239: mol_type = other DNA
atgaaaatgcacctgcagagagctctggtcgtgctggccctgctgaacttcgccaccgtatccctgagcctgtctacatgcaccaccctggatttcggccacatcaagaaaaaacgggtcgaggccatcagaggtcagatcctgagcaagctgaggctgaccagccctccagagcctaccgtgatgacccacgtgccttaccaggtgctggccctgtataattctacaagagagctgctggaagagatgcacggagaaagagaggaaggctgtacccaggagaataccgagagcgagtactacgccaaggaaatccacaagttcgacatgatccagggcctggctgaacataacgagctggccgtgtgccctaagggcatcaccagcaaggtgttcagattcaacgtgtccagcgtggaaaagaacagaacaaacctgtttcgggccgagttccgggtgctgagagtgcccaatccttcttccaagcggaacgagcagagaatcgaactgttccaaatcctgagacccgatgagcacatcgctaaacagagatacatcggcggcaagaacctgcccacacggggcaccgccgagtggctgagctttgatgtgaccgacaccgtgagagagtggctgctgcggcgggagagcaacctgggcctcgaaatcagcatccactgcccttgccacacattccagcctaacggcgacatcctggaaaacatccacgaggtgatggaaattaagtttaagggcgtggacaacgaggacgaccacggccgcggcgatctgggcagactgaagaagcagaaggaccaccacaacccccacctgattctcatgatgatccctccacacagactggataaccctggacagggcggacaaagaaagaagagagccctggacaccaactactgcttccggaacctggaagagaactgctgcgtgcggccactgtacatcgacttcagacaggatctgggatggaagtgggtgcacgagcccaaaggctactacgctaacttctgctcaggcccttgcccttacctgagaagcgccgacacaacccacagcacagtgctgggactgtacaacaccctgaacccagaggccagcgccagcccctgttgtgtgccacaggacctcgagcctctgacaatcctgtactatgtgggcagaacccctaaggtcgaacagctgtctaatatggtggtgaagtcttgtaaatgcagctga

SEQ ID NO: 27 - DNA, 1170 bp, Homo sapiens
  source 1..1170: mol_type = genomic DNA
ctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagctga

SEQ ID NO: 28 - DNA, 1239 bp, Homo sapiens
  source 1..1239: mol_type = genomic DNA
atgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagctga

SEQ ID NO: 29 - protein, 389 aa, Homo sapiens
LSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 30 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 31 - DNA, 1005 bp, synthetic construct
  source 1..1005: mol_type = other DNA
agctaccccatctggtggtccctcgctgtgggccctcagtacagctctctgggatctcagcctatcctgtgcgccagcatccctggcctggtccccaagcagctgagattctgcagaaactacgtggaaatcatgcccagcgtggcagaaggcatcaagatcggaattcaggagtgccaacatcaattcagaggccggcgctggaactgcacaacagtgcacgacagcctggccatcttcggccctgtgctggataaggccaccagggagtcagctttcgtgcacgcaatcgcatcagcaggtgtagcctttgcggtgaccagaagctgtgcagagggcacagccgctatctgtggctgtagcagccggcaccagggcagccccggcaagggctggaagtggggaggttgtagcgaggacatcgagttcggcggcatggtttctagagaattcgccgacgctagagagaaccggccagatgcccgcagcgccatgaacagacacaacaacgaggccggacggcaggccatcgcctctcatatgcacctgaagtgcaagtgccacggcctgtctggcagctgcgaggtgaagacctgctggtggagccagcctgattttcgggccatcggcgacttcctgaaagacaagtacgacagcgccagcgagatggtcgtggagaagcacagagaaagcagaggatgggtggaaaccctgcggcctagatacacctacttcaaggtgcctaccgagcgggacctggtgtactacgaggccagccctaatttctgtgaacccaatccagagaccggcagcttcggaacaagagatagaacatgcaacgtgtccagccacggcatcgacggctgcgacctgctttgctgcggcagaggccacaacgccagagctgaaagaagacgggaaaaatgtagatgcgtgttccactggtgctgctacgtgtcctgccaggaatgtacacgggtgtatgatgtgcacacctgcaaatga

SEQ ID NO: 32 - DNA, 1059 bp, synthetic construct
  source 1..1059: mol_type = other DNA
atggcccctctgggctactttctgctgctgtgctccctgaagcaggccctgggcagctaccccatctggtggtccctcgctgtgggccctcagtacagctctctgggatctcagcctatcctgtgcgccagcatccctggcctggtccccaagcagctgagattctgcagaaactacgtggaaatcatgcccagcgtggcagaaggcatcaagatcggaattcaggagtgccaacatcaattcagaggccggcgctggaactgcacaacagtgcacgacagcctggccatcttcggccctgtgctggataaggccaccagggagtcagctttcgtgcacgcaatcgcatcagcaggtgtagcctttgcggtgaccagaagctgtgcagagggcacagccgctatctgtggctgtagcagccggcaccagggcagccccggcaagggctggaagtggggaggttgtagcgaggacatcgagttcggcggcatggtttctagagaattcgccgacgctagagagaaccggccagatgcccgcagcgccatgaacagacacaacaacgaggccggacggcaggccatcgcctctcatatgcacctgaagtgcaagtgccacggcctgtctggcagctgcgaggtgaagacctgctggtggagccagcctgattttcgggccatcggcgacttcctgaaagacaagtacgacagcgccagcgagatggtcgtggagaagcacagagaaagcagaggatgggtggaaaccctgcggcctagatacacctacttcaaggtgcctaccgagcgggacctggtgtactacgaggccagccctaatttctgtgaacccaatccagagaccggcagcttcggaacaagagatagaacatgcaacgtgtccagccacggcatcgacggctgcgacctgctttgctgcggcagaggccacaacgccagagctgaaagaagacgggaaaaatgtagatgcgtgttccactggtgctgctacgtgtcctgccaggaatgtacacgggtgtatgatgtgcacacctgcaaatga

SEQ ID NO: 33 - DNA, 1005 bp, Homo sapiens
  source 1..1005: mol_type = genomic DNA
agctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtag

SEQ ID NO: 34 - DNA, 1059 bp, Homo sapiens
  source 1..1059: mol_type = genomic DNA
atggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtag

SEQ ID NO: 35 - protein, 334 aa, Homo sapiens
SYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 36 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

(19) United States
(12) Patent Application Publication
(10) Pub. No.: US 2026/0021143 A1
(43) Pub. Date: Jan. 22, 2026

(54) Treatment of osteoarthritis and/or rheumatoid arthritis with pro-chondrogenic and/or chondrocyte protective factors

(71) Applicant: SmartCella Solutions AB, Tullinge (SE)

(72) Inventors: Ran YANG, Solna (SE); Kylie Foo, Stockholm (SE); Eduarde Rohner, Göteborg (SE)

(21) Appl. No.: 19/272,704

(22) Filed: Jul. 17, 2025

(30) Foreign Application Priority Data
Jul. 18, 2024 (EP) 24189402.1
Jul. 18, 2024 (EP) 24189403.9
Jul. 18, 2024 (EP) 24189404.7
Jul. 18, 2024 (EP) 24189406.2
Jul. 18, 2024 (EP) 24189408.8
Jul. 18, 2024 (EP) 24189409.6
Jun. 24, 2025 (EP) 25184805.7
Jun. 24, 2025 (EP) 25184812.3

(51) Int. Cl. A61K 35/28; A61P 19/02; A61P 25/02; C07K 14/47; C07K 14/495; C07K 14/51; C07K 14/54; C07K 14/65; C07K 14/715; C12N 5/00; C12N 5/0775

(52) U.S. Cl. CPC A61K 35/28; A61P 19/02; A61P 25/02; C07K 14/4702; C07K 14/495; C07K 14/51; C07K 14/5428; C07K 14/65; C07K 14/7155; C12N 5/0018; C12N 5/0662; C07K 2319/02; C12N 2501/105; C12N 2501/135; C12N 2501/415; C12N 2501/60

(57) ABSTRACT

The present disclosure relates to mesenchymal stem cells (MSCs) for use in a method of treatment of arthritis. The MSCs are transfected with an mRNA construct encoding a pro-chondrogenic and/or chondrocyte protective factor. The pro-chondrogenic and/or chondrocyte protective factor can be TGFβ3.

### TECHNICAL FIELD

The present disclosure relates to mesenchymal stem cells (MSCs) for use in a method of treatment of arthritis. The MSCs are transfected with an mRNA construct encoding a pro-chondrogenic and/or chondrocyte protective factor. The pro-chondrogenic and/or chondrocyte protective factor can be TGFβ3.

### REFERENCE TO SEQUENCE LISTING

The instant application contains a Sequence Listing which has been submitted electronically in xml format and is hereby incorporated by reference in its entirety. Said xml copy, created on Jul. 17, 2025 is named KIJ-008_SL and is 55,916 bytes in size.

### BACKGROUND

Osteoarthritis (OA) is characterized by a progressive loss of articular cartilage accompanied by new bone formation and synovial proliferation that may culminate in pain, loss of joint function, and disability. Risk factors such as age, sex, trauma, overuse, genetics, and obesity can each make contributions to the process of injury in different compartments of the joint. Such risk factors can serve as initiators that promote abnormal biochemical processes involving the cartilage, bone, and synovium, which over a period of years result in the characteristic features of OA: degradation of articular cartilage, osteophyte formation, subchondral sclerosis, meniscal degeneration, bone marrow lesions, and synovial proliferation (Abramson et al., 2009).

The aging population and the obesity epidemic contribute to the steady increase of OA prevalence observed worldwide. However, the current pharmaceutical treatments recommended by international guidelines to treat OA are merely symptomatic, and are also characterized by relatively small effect sizes and uncertainties around their long-term efficacy and safety. Therefore, there is a need for safe pharmaceutical treatments that can not only treat symptoms but also slows down or even reverts the progression of OA.

Mesenchymal stem cells (MSCs) are found in various tissues, including fat, bone marrow, the umbilical cord, and placental tissue. They are capable of differentiating into mesodermal lineage cells. MSC transplantation has been regarded as a promising approach for OA treatment while engrafting cells alone might not be adequate for effective regeneration. Genetic modification has been used to optimize MSC-based therapy; however, there are still significant limitations that prevent the clinical translation of this therapy including low efficacy and safety concerns (Zhu et al., 2021).

### SUMMARY

Based on the absence of safe and effective treatments, there is an unmet need for safe and effective methods of treatments for arthritis. Thus, the disclosure provides MSCs as the method of treatment for arthritis. Thereby, the MSCs are transfected with mRNA encoding a pro-chondrogenic and/or chondrocyte protective factor, such as TGFβ3. This approach overcomes the limitations of the prior art, since the treatment is more effective compared to non-transfected MSC transplantation and safer compared to transplantation of MSCs that are genetically modified through e.g. viral vectors. Furthermore, the MSCs of this invention provide a stable and long-term expression of the pro-chondrogenic and/or chondrocyte protective factor.

Specifically, an advantage of the here disclosed MSCs for the treatment of arthritis is the prolong duration of the mRNA in the MSCs and the prolong duration of the expression of the pro-chondrogenic and/or chondrocyte protective factor. Therefore, less frequent injections of MSCs into the joints of patients are required to achieve the desired effects of the proliferation of chondrocytes and differentiation of MSCs to chondrocytes.

A first aspect of the disclosure relates to mesenchymal stem cells (MSCs) for use in a method of treatment of arthritis, wherein the MSCs are transfected with an mRNA construct encoding a pro-chondrogenic and/or chondrocyte protective factor.

A second aspect of the disclosure relates to a method of generating MSCs as described herein, wherein the method comprises:
a) providing MSCs; and
b) transfecting the MSCs with an mRNA construct encoding TGFβ3 of SEQ ID NO: 29 or a functional homologue of said TGFβ3 sharing at least 70%, such as at least 80%, for example at least 90%, such as at least 95% sequence identity therewith.

A third aspect of the disclosure relates to a composition comprising MSCs as described herein.

A fourth aspect of the disclosure relates to a method of expressing TGFβ3, the method comprising introducing an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 25 into MSCs as described herein by a non-endosomal pathway of delivery.

A fifth aspect of the disclosure relates to an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 25.

A sixth aspect of the disclosure relates to an engineered MSC comprising an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 25.

### DESCRIPTION OF DRAWINGS

FIG. 1: Quantification of TGFβ3 mRNA for up to 7 days after electroporation. The modRNA level was stable on the first day after electroporation and decreased gradually in the following 6 days. At the end of the 7-day course, the iMSCs still maintained a certain level of modRNA.

FIG. 2: Quantification of TGFβ3 protein products for up to 7 days after electroporation. The protein products of the modRNA were first detected at 6 hours post transfection (6hpt). The protein level increased to reach a plateau from 18 hpt. The expression level declined from 3dpt but remained detectable at the end of the experimental time point (15 pg/1000 cells).

FIG. 3: Quantification of TGFβ3 mRNA in cultured media for up to 7 days after electroporation from cells remained in cultured following electroporation (fresh) or thawed from cryopreservation (frozen). The mRNA level of the TGFβ3 mRNA was comparable across all timepoints after the freeze-and-thaw cycle compared to freshly prepared (never been frozen) iMSCs.

FIG. 4: Quantification of TGFβ3 protein products for up to 4 days after electroporation with cells remained in cultured following electroporation (fresh) or thawed from cryopreservation (frozen). The TGFβ3 expression was lower across all timepoints after the freeze-and-thaw cycle compared to never been frozen iMSCs.

FIG. 5: Immunofluorescence staining indicates an upregulation of aggrecan expression of human articular chondrocytes when co-cultured with iMSCs electroporated with TGFβ3 modRNA. Human articular chondrocytes were co-cultured with iMSCs alone or iMSCs electroporated with TGFβ3 for 3 days (upper panel) or 7 days (lower panel).

FIG. 6: Bar graph of the quantification of 5-Ethynyl-2′-deoxyuridine (EdU) positive cells in human articular chondrocytes. Since EdU is incorporated into the DNA of dividing cells, this shows that iMSCs electroporated with TGFβ3 upregulate the proliferation of human articular chondrocytes.

FIGS. 7A-7B: FIG. 7A Pain assessment of surgical induced OA rat models with or without cell injection. This data shows that iMSCs electroporated with TGFβ3 modRNA can significantly reduce the joint pain in osteoarthritic rats and improve the joint function. FIG. 7B Comparison of therapeutic efficacy between different doses of administered iMSCs. Rats received either a low dose (1 million cells) or a high dose (3 million cells) of TGFβ3 modRNA-transfected iMSCs. The results indicate that both cell doses produce comparable effects on the reduction of joint pain.

FIGS. 8A-8C: FIG. 8A Cartilage histology staining demonstrated a single low dose administration of iMSC+ modTGFβ3 reduced cartilage degeneration from 3 weeks and effect is sustained until end of study in 7 weeks. FIG. 8B Single low dose of iMSC+ modTGFβ3 showed a 42% structural improvement in the cartilage volume (zone 2) after 7 weeks. FIG. 8C Mild structural improvement in both iMSC+WNT3a or TGFβ3 mRNA treated groups in the most severely damaged and the least damaged region.

FIG. 9: TGFβ3 mRNA inside cells after certain time of electroporation. Ctrl is control group without transfection, while 3 hours, 1 week and 2 weeks represent 3 hours, 1 week and 2 weeks after transfection respectively.

### DETAILED DESCRIPTION

### Definitions

As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. Preferably, the method of administration is delivery to a joint of a subject.

Arthritis is defined as diseases of joint disorders that involve inflammation caused by various factors. Osteoarthritis, the most common form of arthritis, is a disease characterized by a slow degenerative processes in the articular cartilage, subchondral bone associated with marginal osteophyte formation, and low grade inflammation. Osteoarthritis is believed to affect 15% of the population in its chronic form. Of those, one-quarter are severely disabled. Most cases of osteoarthritis have no known cause and are referred to as primary osteoarthritis. When the cause of the osteoarthritis is known, the condition is referred to as secondary osteoarthritis. Secondary osteoarthritis is caused by another disease or condition. Conditions that can lead to secondary osteoarthritis include repeated trauma or surgery to the joint structures, abnormal joints at birth (congenital abnormalities), gout, diabetes, and other hormone disorders. Other forms of arthritis are systemic illnesses, such as rheumatoid arthritis and systemic lupus erythematosus (SLE). As used herein “rheumatoid arthritis” refers to a chronic, systemic inflammatory disorder that may affect many tissues and organs, but principally attacks flexible (synovial) joints.

As used herein, the term “anti-inflammatory factor” refers to a naturally occurring or recombinant protein, analogue thereof or fragment thereof that elicits an anti-inflammatory response in a subject. Anti-inflammatory factors of the invention can be Interleukin-10 (IL-10) or Interleukin-1 receptor antagonist (IL1RN).

As used herein, the term “pro-chondrogenic and/or chondrocyte protective factor” refers to a naturally occurring or recombinant protein, analogue thereof or fragment thereof that elicits a pro-chondrogenic and/or chondrocyte protective response in a subject. Pro-chondrogenic and/or chondrocyte protective factors of the invention can be BMP6, IGF1, TGFβ3 or WNT3a.

The term “functional homologue” of an amino acid sequence, refers to a polypeptide comprising said amino acid sequence with the proviso that one or more amino acids are substituted, deleted, added, and/or inserted, and which polypeptide has (qualitatively) the same functionality. Preferably, a functional homologue shares at least 70% sequence identity, preferably at least 80%, preferably at least 85% sequence identity, preferably at least 90% sequence identity, preferably at least 95% sequence identity, more preferred at least 98% sequence identity to said amino acid sequence.

As used herein, a “mesenchymal stem cell” or “MSC” means a mesenchymal cell possessing the ability of differentiating into mesenchymal cells of one or more types and the ability of self-replication. Mesenchymal stem cells are able to differentiate into osteoblasts, chondrocytes, myoblasts, adipocytes, stroma cells, tendon cells, and the like, as with mesodermal cells. The MSCs of this invention can be autologous MSCs from a mammal. The MSCs of this invention can be differentiated from human induced pluripotent stem cells (hiPSCs). The medium for differentiating the hiPSCs to MSCs can be a two-step medium comprising in the first step a WNT pathway agonist and BET pathway antagonist and in the second step a PDGF pathway agonist, an IGF1 pathway agonist and an FGF-beta pathway agonist. For MSCs that are differentiated from hiPSCs with said aforementioned two step medium, the term “induced MSCs” or “iMSCs” is used herein.

As used herein the term “mRNA construct” comprises a coding region. Furthermore, the “mRNA construct” can comprise 5′UTR, 3′UTR, Poly(a) tail, 5′cap and a region encoding a signal peptide. The mRNA construct can consist of chemically unmodified nucleotide bases or can comprise chemically modified nucleotide bases. Furthermore, the mRNA construct can be codon optimized as described in the chapter “mRNA construct”. The region coding for a signal peptide can code for the native signal peptide of the encoded protein of the mRNA or a heterologous signal peptide. The mRNA construct can be purified.

As used herein, the term “pain” comprises any sensory and/or emotional experience that may arise from actual or potential tissue damage, including but not limited to nociceptive, neuropathic, and/or inflammatory pain. Pain may be acute or chronic and may manifest locally, regionally, or systemically. The term encompasses both subjective reports of discomfort and objectively measurable responses such as behavioural or physiological indicators. Pain may, for example, be associated with conditions such as arthritis, injury, or degenerative disease and may include symptoms such as joint tenderness, reduced mobility, stiffness, and hypersensitivity to stimuli (hyperalgesia or allodynia). Pain may be quantified or assessed using standardised and validated clinical tools. Such tools include, but are not limited to, physical examinations and patient-reported assessment tools. Physical examinations that provide appropriately directed neurological and musculoskeletal evaluation include, but are not limited to selective nerve root block, medial branch block, provocative discography, facet joint injection and sacroiliac joint injection. Other tool including the Visual Analogue Scale (VAS), the Numerical Rating Scale (NRS), the McGill Pain Questionnaire (MPQ), and the Brief Pain Inventory (BPI). These tools provide subjective scoring based on patient self-report, as well as objective assessments including behavioural or functional indicators. Such assessments may be used to evaluate the severity, duration, interference with daily activities, and qualitative characteristics of pain.

The term “polypeptide” or “protein” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

The term “sequence identity” as used herein describes the relatedness between two amino acid sequences or between two nucleotide sequences, i.e. a candidate sequence (e.g. a mutant sequence) and a reference sequence (such as a wild type sequence) based on their pairwise alignment. For purposes of the present invention, the sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277,), preferably version 5.0.0 or later (available at https://www.ebi.ac.uk/Tools/psa/emboss_needle/). The parameters used are gap open penalty of 10, gap extension penalty of 0.5, and the EBLOSUM62 (EMBOSS version of BLOSUM62) substitution matrix. The output of Needle labelled “longest identity” (obtained using the −nobrief option) is used as the percent identity and is calculated as follows:

(Identical Residues×100)/(Length of Alignment−Total Number of Gaps in Alignment)

The Needleman-Wunsch algorithm is also used to determine whether a given amino acid in a sequence other than the reference sequence corresponds to a given position in a reference sequence. For purposes of the present invention, the sequence identity between two nucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the DNAFULL (EMBOSS version of NCBI NUC4.4) substitution matrix. The output of Needle labelled “longest identity” (obtained using the −nobrief option) is used as the percent identity and is calculated as follows:

(Identical Deoxyribonucleotides×100)/(Length of Alignment−Total Number of Gaps in Alignment).

As used herein “signal peptide” refers to a short (less than 60 amino acids, for example, 3 to 60 amino acids) polypeptide present on precursor proteins (typically at the N terminus), and which is typically absent from the mature protein. The signal peptide directs the transport and/or secretion of the translated protein through the membrane. Signal peptides may also be called targeting signals, transit peptides, localization signals, or signal sequences. For example, the signal sequence may be a co-translational or post-translational signal peptide. The signal peptide may be, e.g., the native signal peptide of the protein to be produced, a heterologous signal peptide, or a hybrid of native and heterologous signal peptide. Numerous signal peptides are used for production of secreted proteins.

As used herein, the term “subject” includes humans and mammals (e.g., mice, rats, rabbits, pigs, cats, dogs, and horses). In preferred embodiments, subjects are mammals, particularly primates, especially humans. In some embodiments the subjects are patients.

The term “transfection” refers to the introduction of nucleic acid molecules, such as DNA or RNA (e.g. mRNA) molecules, into cells, preferably into eukaryotic cells. In the context of the present disclosure, the term “transfection” encompasses any method known to the skilled person for introducing nucleic acid molecules into cells, preferably into eukaryotic cells, such as into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. Preferably, the introduction is non-viral.

As used herein, the terms “treat,” “treating” and “treatment,” contemplate an action that occurs while a patient is suffering from or susceptible to a specified disease, disorder or condition, which delays onset of and/or reduces the frequency or severity of one or more symptoms or features of the disease disorder or condition. Thus, “treat”, “treating”, and “treatment” refer to any type of treatment that imparts a benefit to a subject afflicted with a disease, disorder or condition, including improvement in the condition of the subject (e.g., in one or more symptoms), delay in the progression of the disease, disorder or condition, prevention or delay of the onset of the disease, disorder or condition, etc. In the context of arthritis, the term “treatment” may comprise, for example, alleviating pain associated with the arthritic condition, restoring joint function, or inhibiting cartilage degradation. The treatment may also include modulation of the inflammation and/or immune response, reduction in joint effusion, stabilization of bone structure, or promotion of tissue regeneration, such as through the enhancement of chondrogenesis or the reconstitution of the synovial microenvironment.

### Mesenchymal Stem Cells for Use in a Method of Treatment

The disclosure provides MSCs for treatment of arthritis. Thereby, the MSCs are transfected with mRNA encoding a pro-chondrogenic and/or chondrocyte protective factor, such as TGFβ3. This approach overcomes the limitations of the prior art, since the treatment is more effective compared to non-transfected MSC transplantation and safer compared to transplantation of MSCs that are genetically modified through e.g. viral vectors.

Thus, in a main aspect, the present disclosure relates to mesenchymal stem cells (MSCs) for use in a method of treatment of arthritis, wherein the MSCs are transfected with an mRNA construct encoding a pro-chondrogenic and/or chondrocyte protective factor.

In another aspect, the present disclosure relates to a composition comprising MSCs as described herein.

### TGFβ3

TGF3β is a cytokine, which is involved in cell differentiation, embryogenesis and development. TGF3β also plays an essential role in controlling the development of lungs in mammals, by also regulating cell adhesion and ECM formation in this tissue, and controls wound healing by regulating the movements of epidermal and dermal cells in injured skin.

Thus, in some embodiments of the present disclosure, the mRNA construct comprises mRNA encoding an TGFβ3 of SEQ ID NO: 29 or a functional homologue of said TGFβ3 sharing at least 70%, such as at least 80%, for example at least 90%, such as at least 95% sequence identity therewith. In some embodiments, the mRNA construct comprises mRNA encoding an TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, for example at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In some embodiments of the present disclosure, the mRNA construct comprises mRNA encoding an TGFβ3 of SEQ ID NO: 30 or a functional homologue of said TGFβ3 sharing at least 70%, such as at least 80%, for example at least 90%, such as at least 95% sequence identity therewith. In some embodiments of the present disclosure, the mRNA construct comprises mRNA encoding an TGFβ3 of SEQ ID NO: 30 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, for example at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In some embodiments of the present disclosure, the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 25. In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, for example at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 25.

In some embodiments of the present disclosure, the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 26. In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, for example at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 26.

In some embodiments of the present disclosure, the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 27. In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, for example at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 27.

In some embodiments of the present disclosure, the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 28. In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, for example at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 28.

In another aspect, the present disclosure relates to a method of expressing TGFβ3, the method comprising introducing an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 25 into MSCs as described herein by a non-endosomal pathway of delivery. In some embodiments of the present disclosure, the mRNA construct introduced into the MSCs comprises mRNA having at least 70%, such as at least 80%, such as at least 85%, such as at least 90%, such as at least 95%, such as at least 97%, such as at least 99%, such as 100% sequence identity to SEQ ID NO: 26. In some embodiments of the present disclosure, the mRNA construct introduced into the MSCs comprises mRNA having at least 70%, such as at least 80%, such as at least 85%, such as at least 90%, such as at least 95%, such as at least 97%, such as at least 99%, such as 100% sequence identity to SEQ ID NO: 27. In some embodiments of the present disclosure, the mRNA construct introduced into the MSCs comprises mRNA having at least 70%, such as at least 80%, such as at least 85%, such as at least 90%, such as at least 95%, such as at least 97%, such as at least 99%, such as 100% sequence identity to SEQ ID NO: 28. In another aspect, the present disclosure relates to a method of expressing TGFβ3, the method comprising introducing an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 25 into MSCs as described herein by a non-endosomal pathway of delivery. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 26. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 27. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 28

In another aspect, the present disclosure relates to an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 25. In another aspect, the present disclosure relates to an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 26. In another aspect, the present disclosure relates to an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 25. In another aspect, the present disclosure relates to an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 26.

In some embodiments of the present disclosure, the activity of TGFβ3 is determined via immunoassays, such as ELISA or AlphaLISA. In some embodiments of the present disclosure, the amount of TGFβ3 in the supernatant of the MSCs in cell culture is determined via immunoassays, such as ELISA or AlphaLISA.

### IGF1

IGF1 is mainly secreted by the liver as a result of stimulation by growth hormone (GH). IGF1 is important for both the regulation of normal physiology, as well as a number of pathological states, including cancer. The IGF axis has been shown to play a role in the promotion of cell proliferation and the inhibition of cell death (apoptosis).

Thus, in some embodiments of the present disclosure, the mRNA construct comprises mRNA encoding an IGF1 of SEQ ID NO: 23 or a functional homologue of said IGF1 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In some embodiments of the present disclosure, the mRNA construct comprises mRNA encoding an IGF1 of SEQ ID NO: 24 or a functional homologue of said IGF1 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 19.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 20.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 21.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 22.

In another aspect, the present disclosure relates to a method of expressing IGF1, the method comprising introducing an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 19 into MSCs as described herein by a non-endosomal pathway of delivery. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 20. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 21. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 22.

In another aspect, the present disclosure relates to an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 19. In another aspect, the present disclosure relates to an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 20.

In some embodiments of the present disclosure, the activity of IGF1 is determined via immunoassays, such as ELISA or AlphaLISA. In some embodiments of the present disclosure, the amount of IGF1 in the supernatant of the MSCs in cell culture is determined via immunoassays, such as ELISA or AlphaLISA.

### BMP6

BMPs are known for their ability to induce the growth of bone and cartilage. The bone morphogenetic proteins (BMPs) are a family of secreted signaling molecules that can induce ectopic bone growth. BMP6 is able to induce all osteogenic markers in mesenchymal stem cells.

Thus, in some embodiments of the present disclosure, the mRNA construct comprises mRNA encoding an BMP6 of SEQ ID NO: 17 or a functional homologue of said BMP6 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In some embodiments of the present disclosure, the mRNA construct comprises mRNA encoding an BMP6 of SEQ ID NO: 18 or a functional homologue of said BMP6 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 13.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 14.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 15.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 16.

In another aspect, the present disclosure relates to a method of expressing BMP6, the method comprising introducing an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 13 into MSCs as described herein by a non-endosomal pathway of delivery. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 14. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 15. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 16.

In another aspect, the present disclosure relates to an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 13. In another aspect, the present disclosure relates to an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 14.

In some embodiments of the present disclosure, the activity of BMP6 is determined via immunoassays, such as ELISA or AlphaLISA. In some embodiments of the present disclosure, the amount of BMP6 in the supernatant of the MSCs in cell culture is determined via immunoassays, such as ELISA or AlphaLISA.

### IL-10

Interleukins (ILs) are a group of cytokines (secreted proteins and signal molecules) that are expressed and secreted by white blood cells (leukocytes) as well as some other body cells. Interleukin 10 (IL-10) is a protein that inhibits the synthesis of a number of cytokines, including IFN-gamma, IL-2, IL-3, TNF, and GM-CSF produced by activated macrophages and by helper T-cells. The terms “IL-10”, “IL10” and “Interleukin 10” are used interchangeably throughout the description. In arthritis, activated CD4 T helper cells and macrophages are believed to be the primary driving force behind joint inflammation. IL-10 can inhibit the synthesis of cytokines produced by said CD4 T-helper cells and macrophages.

Thus, in some embodiments of the present disclosure, the mRNA construct comprises mRNA encoding an IL-10 of SEQ ID NO: 5 or a functional homologue of said IL-10 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In some embodiments of the present disclosure, the mRNA construct comprises mRNA encoding an IL-10 of SEQ ID NO: 6 or a functional homologue of said IL-10 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 1.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 2.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 3.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 4.

In another aspect, the present disclosure relates to a method of expressing IL-10, the method comprising introducing an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 1 into MSCs as described herein by a non-endosomal pathway of delivery. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 2. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 3. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 4.

In another aspect, the present disclosure relates to an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 1. In another aspect, the present disclosure relates to an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 2.

In some embodiments of the present disclosure, the activity of IL-10 is determined via immunoassays, such as ELISA or AlphaLISA. In some embodiments of the present disclosure, the amount of IL-10 in the supernatant of the MSCs in cell culture is determined via immunoassays, such as ELISA or AlphaLISA.

### IL1RN

IL1RN is a member of the interleukin 1 cytokine family. IL1RN is secreted by various types of cells including immune cells, epithelial cells, and adipocytes, and is a natural inhibitor of the pro-inflammatory effect of IL1β. The terms “IL1RN”, “IL-1RA” and “Interleukin-1 receptor antagonist” are used interchangeably throughout the description.

Thus, in some embodiments of the present disclosure, the mRNA construct comprises mRNA encoding an IL1RN of SEQ ID NO: 11 or a functional homologue of said IL1RN sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In some embodiments of the present disclosure, the mRNA construct comprises mRNA encoding an IL1RN of SEQ ID NO: 12 or a functional homologue of said IL1RN sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 7.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 8.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 9.

In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 10.

In another aspect, the present disclosure relates to a method of expressing IL1RN, the method comprising introducing an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 7 into MSCs as described herein by a non-endosomal pathway of delivery. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 8. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 9. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 10.

In another aspect, the present disclosure relates to an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 7. In another aspect, the present disclosure relates to an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 8.

In some embodiments of the present disclosure, the activity of IL-10 is determined via immunoassays, such as ELISA or AlphaLISA. In some embodiments of the present disclosure, the amount of IL-10 in the supernatant of the MSCs in cell culture is determined via immunoassays, such as ELISA or AlphaLISA.

### WNT3a

WNT3a is highly related to the WNT3 gene in sequence and protein function. WNT3a and WNT3 signal similarly through primarily the beta-catenin/Tcf pathway. WNT proteins are critical in tissue homeostasis, embryonic development, and disease.

Thus, in some embodiments of the present disclosure, the mRNA construct comprises mRNA encoding an WNT3a of SEQ ID NO: 35 or a functional homologue of said WNT3a sharing at least 70%, such as at least 80%, for example at least 90%, such as at least 95% sequence identity therewith. In some embodiments, the mRNA construct comprises mRNA encoding an WNT3a of SEQ ID NO: 35 or a functional homologue of said WNT3a sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, for example at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In some embodiments of the present disclosure, the mRNA construct comprises mRNA encoding an WNT3a of SEQ ID NO: 36 or a functional homologue of said WNT3a sharing at least 70%, such as at least 80%, for example at least 90%, such as at least 95% sequence identity therewith. In some embodiments, the mRNA construct comprises mRNA encoding an WNT3a of SEQ ID NO: 36 or a functional homologue of said WNT3a sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, for example at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In some embodiments of the present disclosure, the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 31. In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 31.

In some embodiments of the present disclosure, the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 32. In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 32.

In some embodiments of the present disclosure, the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 33. In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 33.

In some embodiments of the present disclosure, the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 34. In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity to SEQ ID NO: 34.

In another aspect, the present disclosure relates to a method of expressing WNT3a, the method comprising introducing an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 31 into MSCs as described herein by a non-endosomal pathway of delivery. In some embodiments of the present disclosure, the mRNA construct introduced into the MSCs comprises mRNA having at least 70%, such as at least 80%, such as at least 85%, such as at least 90%, such as at least 95%, such as at least 97%, such as at least 99%, such as 100% sequence identity to SEQ ID NO: 32. In some embodiments of the present disclosure, the mRNA construct introduced into the MSCs comprises mRNA having at least 70%, such as at least 80%, such as at least 85%, such as at least 90%, such as at least 95%, such as at least 97%, such as at least 99%, such as 100% sequence identity to SEQ ID NO: 33. In some embodiments of the present disclosure, the mRNA construct introduced into the MSCs comprises mRNA having at least 70%, such as at least 80%, such as at least 85%, such as at least 90%, such as at least 95%, such as at least 97%, such as at least 99%, such as 100% sequence identity to SEQ ID NO: 34. In another aspect, the present disclosure relates to a method of expressing WNT3a, the method comprising introducing an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 31 into MSCs as described herein by a non-endosomal pathway of delivery. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 32. In some embodiments of the present disclosure, the mRNA construct introduced into the MSCs comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 34.

In another aspect, the present disclosure relates to an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 31. In another aspect, the present disclosure relates to an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 32. In another aspect, the present disclosure relates to an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 31. In another aspect, the present disclosure relates to an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 32.

In some embodiments of the present disclosure, the activity of WNT3a is determined via immunoassays, such as ELISA or AlphaLISA. In some embodiments of the present disclosure, the amount of WNT3a in the supernatant of the MSCs in cell culture is determined via immunoassays, such as ELISA or AlphaLISA.

### Signal Peptide

The disclosure regards MSCs expressing pro-chondrogenic and/or chondrocyte protective factors, such as TGFβ3. The pro-chondrogenic and/or chondrocyte protective factor can be secreted by the MSCs into the joints of a subject. Therefore, the factor can have a signal peptide directing the transport and/or secretion of the translated protein through the membrane. The signal peptide may be, e.g., the native signal peptide of the protein to be produced, a heterologous signal peptide, or a hybrid of native and heterologous signal peptide. Numerous signal peptides are used for production of secreted proteins. The mRNA construct used in the methods and compositions of the disclosure can encode a secreted protein. As used herein, a “secreted protein” refers to a protein that is made inside a cell (intracellularly) and then is released outside the cell (extracellularly). The term “secreted protein” is intended to encompass any protein that comprises a signal peptide that directs the protein toward the secretory pathway as well as proteins that are non-classically secreted. Bioinformatics tools exist that can predict secretion via one or the other mechanism with very high likelihood. In an embodiment, the secreted protein comprises a signal peptide that has been engineered into the protein (e.g., a heterologous signal peptide that directs the protein toward the secretory pathway). Numerous classes of secreted proteins are well-established in the art, including a wide variety of secreted proteins that have been demonstrated to be useful therapeutically.

In some embodiments, the signal peptide is at least 3 amino acids, such as at least 5 amino acids, such as at least 7 amino acids, such as at least 10 amino acids, such as at least 13 amino acids, such as at least 15 amino acids long. In some embodiments, the signal peptide is at most 60 amino acids, such as at most 58 amino acids, such as at most 55 amino acids, such as at most 53 amino acids, such as at most 50 amino acids long. In some embodiments, the signal peptide is from 3 to 60 amino acids, such as from 5 to 55 amino acids, such as from 7 to 50 amino acids, such as from 10 to 45 amino acids long.

In some embodiments of the present disclosure, the mRNA construct comprises mRNA encoding TGFβ3 of SEQ ID NO: 29 or a functional homologue of said TGFβ3 sharing at least 70%, such as at least 80%, for example at least 90%, such as at least 95% sequence identity therewith and a signal peptide. In some embodiments of the present disclosure, the mRNA construct comprises mRNA encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith linked to a signal peptide.

In some embodiments of the present disclosure, the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 25 and a signal peptide. In some embodiments of the present disclosure, the mRNA construct comprises mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 25 linked to a signal peptide.

In some embodiments of the present disclosure, the signal peptide is a heterologous or native signal peptide.

In some embodiments of the present disclosure, the mRNA construct encodes an TGFβ3 of SEQ ID NO: 30 or a functional homologue of said TGFβ3 sharing at least 70%, such as at least 80%, for example at least 90%, such as at least 95% sequence identity therewith. In some embodiments of the present disclosure, the mRNA construct encodes an TGFβ3 of SEQ ID NO: 30 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith. Thus, in some embodiments, the mRNA construct encodes for TGFβ3 and a signal peptide.

In some embodiments of the present disclosure, the mRNA construct comprises an mRNA construct having at least 70%, such as at least 80%, such as at least 85%, such as at least 90%, such as at least 95%, such as at least 97%, such as at least 99%, such as 100% sequence identity to SEQ ID NO: 26. In some embodiments of the present disclosure, the mRNA construct comprises an mRNA construct having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 26. Thus, in some embodiments, the mRNA construct encodes for TGFβ3 and a signal peptide.

### Mesenchymal Stem Cells

As used herein, the term “mesenchymal stem cell” (MSC) refers to multipotent stem cells established in the art that can self-renew by dividing and can differentiate into multiple tissues including bone, cartilage, muscle cells, fat cells and connective tissue. The term MSC encompasses both natural stem cells that are naturally present in certain tissues and induced MSCs that can be prepared in vitro.

In some embodiments of the present disclosure, the MSCs are derived from adult tissue, such as bone marrow, adipose tissue or peripheral blood. In some embodiments of the present disclosure, the MSCs are derived from prenatal, neonatal or umbilical tissue, such as umbilical cord, amniotic fluid, cord blood, placenta or Wharton's jelly. In some embodiments of the present disclosure, the MSCs are derived from a tissue selected from the group consisting of bone marrow, adipose tissue, peripheral blood, umbilical cord, amniotic fluid, cord blood, placenta and Wharton's jelly. In some embodiments of the present disclosure, the MSCs are induced mesenchymal stem cells. In some embodiments of the present disclosure, the MSCs are derived from stem cells. To the extent that reference to “stem cell” is made herein, this should be understood as a reference to a cell exhibiting multilineage differentiative potential. The stem cell is not obtained by methods that involve the destruction of a human embryo. It is possible to establish embryonic stem cells without destroying human embryos.

In some embodiments of the present disclosure, the MSCs are bone marrow mesenchymal stem cells (BMSCs), which can be directly isolated from subjects.

Although some functional diversity exists within mesenchymal stem cells derived from different subjects and/or different tissue sources, for mesenchymal stem cells to maintain their identity they should possess three functional attributes: 1) self-renewal potential; 2) ability to grow on plastics; and 3) ability to differentiate into three major cell types including osteoblast (bone), chondrocyte (cartilage) and adipocyte (fat). Additionally, regardless of the source of MSCs, the MSCs should have differentiation markers such as CD73, CD90 and the lack of CD14, CD34, and CD45 (Ullah et al., 2015; Fitzsimmons et al. (2018) Stem Cells Int. 2018:8031718).

In some embodiments of the present disclosure, the MSCs are induced MSCs, that have been prepared from pluripotent stem cells, such as human embryonic stem cells (ESCs) or human induced pluripotent stem cells (iPSCs). Methods of preparing iMSCs from pluripotent stem cells have been described in the art (see e.g., Soontararak et al., 2018; Yang et al., 2019; Xu et al., 2019).

In some embodiments of the present disclosure, the MSCs are autologous MSCs from a mammal. In some embodiments of the present disclosure, the MSCs are obtained from a mammalian donor, such as a dog, cat, horse, lion, giraffe, elephant, zebra, chimpanzee, gorilla, rhinoceros, hippopotamus, leopard, bear or human. In some embodiments of the present disclosure, the MSCs are obtained from a human donor. In some embodiments of the present disclosure, the MSCs are freshly-obtained cells. As used herein, both the terms “freshly-obtained cells” and “cryopreserved cells” refer to cells, that are thawed, cultured and where subsequently the mRNA is introduced. As used herein, the term “cryopreserved cells” refers to cells that are cryopreserved comprising the mRNA composition and thawed afterwards for use. As used herein, the term “freshly-obtained cells” refers to cells that are not cryopreserved comprising the mRNA composition.

In some embodiments of the present disclosure, the MSCs are freshly-obtained cells. In other embodiments, the MSCs are cryopreserved cells that are thawed prior to introducing the mRNA construct.

Accordingly, in some embodiments of the present disclosure, the mRNA-loaded MSC are used fresh after mRNA loading. In another embodiment, the mRNA-loaded MSC are cryopreserved after mRNA loading and then thawed prior to use. In some embodiments of the present disclosure, the MSCs are cryopreserved in 10% DMSO. In some embodiments of the present disclosure, the MSCs are cryopreserved in at least 5% DMSO.

### Method of Producing MSCs

The present disclosure shows a surprisingly prolong duration of mRNA in the MSCs and protein expression. This is of advantage for the treatment of arthritis, since less injections of MSCs into the joints of subjects are required to achieve the desired effects of proliferation of chondrocytes and differentiation of MSCs to chondrocytes. One reason for this surprising effect can be the method of generating the MSCs used for the method of treatment.

Thus, in one aspect, the present disclosure relates to a method of generating MSCs as described herein, wherein the method comprises:
a) Providing MSCs; and
b) Transfecting the MSCs with an mRNA construct encoding TGFβ3 of SEQ ID NO: 29 or a functional homologue of said TGFβ3 sharing at least 70%, such as at least 80%, for example at least 90%, such as at least 95% sequence identity therewith.

In some embodiments of this disclosure, the method of generating MSCs as described herein comprises:
a) Providing MSCs; and
b) Transfecting the MSCs with an mRNA construct encoding TGFβ3 of SEQ ID NO: 30 or a functional homologue of said TGFβ3 sharing at least 70%, such as at least 80%, for example at least 90%, such as at least 95% sequence identity therewith.

In some embodiments of this disclosure, the method of generating MSCs as described herein comprises:
a) Providing MSCs; and
b) Transfecting the MSCs with an mRNA construct having at least 70%, such as at least 80%, such as at least 85%, such as at least 90%, such as at least 95%, such as at least 97%, such as at least 99%, such as 100% sequence identity to SEQ ID NO: 25.

In some embodiments of this disclosure, the method of generating MSCs as described herein comprises:
a) Providing MSCs; and
b) Transfecting the MSCs with an mRNA construct having at least 70%, such as at least 80%, such as at least 85%, such as at least 90%, such as at least 95%, such as at least 97%, such as at least 99%, such as 100% sequence identity to SEQ ID NO: 26.

In some embodiments of this disclosure, the method of generating MSCs as described herein comprises:
a) Providing MSCs; and
b) Transfecting the MSCs with an mRNA construct encoding TGFβ3 of SEQ ID NO: 29 or a functional homologue of said TGFβ3 sharing at least 70%, such as at least 80%, for example at least 90%, such as at least 95% sequence identity therewith and a signal peptide.

In some embodiments of the present disclosure, the MSCs in step a) of the method of generating MSCs are cultured for at least 1, such as at least 2, such as at least 5, such as at least 10 passages before transfection. In some embodiments of the present disclosure, the MSCs in step a) of the method of generating MSCs are cultured for at least 1 passage before transfection. In some embodiments of the present disclosure, the MSCs in step a) of the method of generating MSCs are cultured for at least 2 passages before transfection. In some embodiments of the present disclosure, the MSCs in step a) of the method of generating MSCs are cultured for at least 3 passages before transfection. In some embodiments of the present disclosure, the MSCs in step a) of the method of generating MSCs are cultured for at least 4 passages before transfection. In some embodiments of the present disclosure, the MSCs in step a) of the method of generating MSCs are cultured for at least 5 passages before transfection. In some embodiments of the present disclosure, the MSCs in step a) of the method of generating MSCs are cultured for at least 6 passages before transfection. In some embodiments of the present disclosure, the MSCs in step a) of the method of generating MSCs are cultured for at least 7 passages before transfection. In some embodiments of the present disclosure, the MSCs in step a) of the method of generating MSCs are cultured for at least 8 passages before transfection. In some embodiments of the present disclosure, the MSCs in step a) of the method of generating MSCs are cultured for at least 9 passages before transfection. In some embodiments of the present disclosure, the MSCs in step a) of the method of generating MSCs are cultured for at least 10 passages before transfection.

In some embodiments of the present disclosure, the MSCs in step a) of the method of generating MSCs are cultured for at most 10, such as at most 5, such as at most 2, such as at most 1 passage(s) before transfection. In some embodiments of the present disclosure, the MSCs in step a) of the method of generating MSCs are cultured for at most 9 passages before transfection. In some embodiments of the present disclosure, the MSCs in step a) of the method of generating MSCs are cultured for at most 8 passages before transfection. In some embodiments of the present disclosure, the MSCs in step a) of the method of generating MSCs are cultured for at most 7 passages before transfection. In some embodiments of the present disclosure, the MSCs in step a) of the method of generating MSCs are cultured for at most 6 passages before transfection. In some embodiments of the present disclosure, the MSCs in step a) of the method of generating MSCs are cultured for at most 5 passages before transfection. In some embodiments of the present disclosure, the MSCs in step a) of the method of generating MSCs are cultured for at most 4 passages before transfection. In some embodiments of the present disclosure, the MSCs in step a) of the method of generating MSCs are cultured for at most 3 passages before transfection. In some embodiments of the present disclosure, the MSCs in step a) of the method of generating MSCs are cultured for at most 2 passages before transfection. In some embodiments of the present disclosure, the MSCs in step a) of the method of generating MSCs are cultured for at most 1 passage before transfection.

In some embodiments of the present disclosure, passage 0 is the passage from the day where the medium from PSCs maintenance medium is changed to MSC induction medium. In some embodiments of the present disclosure, passage 0 is the passage from the day where the medium from PSCs maintenance medium is changed to medium comprising a WNT pathway agonist and a BET pathway antagonist.

In some embodiments of the present disclosure, the MSCs are generated by
a. culturing human pluripotent stem cells in a culture medium comprising a WNT pathway agonist and a BET pathway antagonist for at least two days to generate induced cells; and
b. culturing the induced cells from step (a) in a culture medium comprising a PDGF pathway agonist, an IGF1 pathway agonist and an FGF-beta pathway agonist for at least ten days.

In some embodiments of the present disclosure, the human pluripotent stem cells are human embryonic stem cells. In some embodiments, the pluripotent stem cells are derived from a mammalian donor, such as a dog, cat, horse, lion, giraffe, elephant, zebra, chimpanzee, gorilla, rhinoceros, hippopotamus, leopard, bear, or human. In some embodiments of the present disclosure, the starting cells used in the cultures of the disclosure are human pluripotent stem cells. As used herein, the term “human pluripotent stem cell” (abbreviated as hPSC) refers to a human stem cell that has the capacity to differentiate into a variety of different cell types. The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, for example, using a nude mouse and teratomas formation assay. Pluripotency can also be evidenced by the expression of embryonic stem(ES) cell markers, although the preferred test for pluripotency is the demonstration of the capacity to differentiate into cells of each of the three germ layers.

Human pluripotent stem cells include, for example, human embryonic stem cells, such as ES cell lines, and induced pluripotent stem cells (iPSC). Human pluripotent stem cells (hPSCs) express cellular markers that can be used to identify cells as being hPSCs. Non-limiting examples of pluripotent stem cell markers include TRA-1-60, TRA-1-81, TRA-2-54, SSEA1, SSEA3, SSEA4, CD9, CD24, OCT3, OCT4, NANOG and/or SOX2. In some embodiments of the present disclosure, the mature iMSCs generated by the method lack expression of the pluripotent stem cell marker TRA-1-60.

The pluripotent stem cells can be subjected to culture conditions, as described herein, that induce cellular differentiation. As used herein, the term “differentiation” refers to the development of a cell from a more primitive stage towards a more mature (i.e., less primitive) cell, typically exhibiting phenotypic features of commitment to a particular cellular lineage. In some embodiments of the present disclosure, progenitors of iMSCs are first generated, followed by further differentiation into mature iMSCs.

In some embodiments of the present disclosure, the mature iMSCs generated by the differentiation methods express mesenchymal surface markers, such as CD73, CD90, CD105, CD29 and/or CD44. In some embodiments of the present disclosure, the iMSCs express one or more mesenchymal surface markers selected from the group consisting of CD73, CD90, CD105, CD29, CD44, and combinations thereof. In some embodiments of the present disclosure, in a population of iMSCs at least 90%, such as at least 95%, such as least 98%, such as at least 99% of live cells express one or more mesenchymal surface markers selected from the group consisting of CD73, CD90, CD105, CD29, CD44, and combinations thereof. In some embodiments of the present disclosure, in a population of iMSCs at least 90%, such as at least 95%, such as least 98%, such as at least 99% of live cells express the mesenchymal surface makers CD73, CD90, CD105, CD29 and CD44.

In some embodiments of the present disclosure, the mature iMSCs generated by the differentiation methods lack expression of at least one pluripotent stem cell marker, such as TRA-1-60 and/or at least one hematopoietic cell marker, such as CD34, CD45 and/or at least one immunogenic marker, such as HLA-DR. In some embodiments of the present disclosure, the mature iMSCs lack expression of one or more markers selected from the group consisting of TRA-1-60, CD11b, CD19, CD34, CD45, HLA-DR, and combinations thereof. In some embodiments of the present disclosure, in a population of iMSCs less than 5%, such as less than 3%, such as less than 2%, such as less than 1% of live cells express one or more surface markers selected from the group consisting of TRA-1-60, CD11b, CD19, CD34, CD45, HLA-DR, and combinations thereof.

In some embodiments of the present disclosure, the method for generating mesenchymal stem cells comprises culturing human pluripotent stem cells in culture medium comprising specific agonist and/or antagonists of cellular receptors and/or signalling pathways.

In some embodiments of the present disclosure, human pluripotent stem cells are cultured for at least two days, or at least three days or for four days (day 0 to day 4) in an induction medium that comprises a WNT pathway agonist and a BET pathway antagonist. In some embodiments of the present disclosure, day 0 is defined as the day when the hiPSCs start to differentiate. In some embodiments of the present disclosure, day 0 is defined as the day when the hiPSC culture medium is changed to iMSC induction medium. In some embodiments of the present disclosure, day 0 is defined as the day when the hiPSC culture medium is changed to medium comprising a WNT pathway agonist and a BET pathway antagonist.

In some embodiments of the present disclosure, the induced cells which were cultured in cell culture medium comprising a WNT pathway agonist and a BET pathway antagonist are further cultured for at least ten days (or at least 11, 12, 13, or 14 days), such as on day 4 to day 14 (following a 4-day induction step) in an enhancement medium comprising a PDGF pathway agonist, an IGF1 pathway agonist and an FGF-beta pathway agonist, which generates iMSC progenitor cells. In some embodiments of the present disclosure, the iMSC progenitor cells are cultured first in cell culture medium comprising a WNT pathway agonist and a BET pathway antagonist and second in an enhancement medium comprising a PDGF pathway agonist, an IGF1 pathway agonist and an FGF-beta pathway agonist. In some embodiments of the present disclosure, the iMSC progenitor cells are further cultured for at least 7 days or more in a maintenance medium comprising an L-glutamine/GlutaMAX supplement to generate mature iMSCs.

As used herein, an “agonist” of a cellular receptor or signaling pathway is intended to refer to an agent that stimulates (upregulates) the cellular receptor or signaling pathway. Stimulation of the cellular signaling pathway can be initiated extracellularly, for example by use of an agonist that activates a cell surface receptor involved in the signaling pathway (e.g., the agonist can be a receptor ligand). Additionally or alternatively, stimulation of cellular signaling can be initiated intracellularly, for example by use of a small molecule agonist that interacts intracellularly with a component(s) of the signaling pathway.

As used herein, an “antagonist” of a cellular signaling pathway is intended to refer to an agent that inhibits (downregulates) the cellular signaling pathway. Inhibition of the cellular signaling pathway can be initiated extracellularly, for example by use of an antagonist that blocks a cell surface receptor involved in the signaling pathway. Additionally or alternatively, inhibition of cellular signaling can be initiated intracellularly, for example by use of a small molecule antagonist that interacts intracellularly with a component(s) of the signaling pathway.

In some embodiments of the present disclosure, agonists of the WNT pathway include agents, molecules, compounds, or substances capable of stimulating (upregulating) the canonical Wnt/β-catenin signaling pathway, which biologically is activated by binding of a Wnt-protein ligand to a Frizzled family receptor. In some embodiments of the present disclosure, a WNT pathway agonist is a glycogen synthase kinase 3 (Gsk3) inhibitor, such as CHIR98014. In some embodiments of the present disclosure, the WNT pathway agonist is selected from the group consisting of CHIR98014, CHIR99021, SB 216763, SB 415286, LY2090314, 3F8, A 1070722, AR-A 014418, BIO, AZD1080, WNT3A, and combinations thereof. In some embodiments of the present disclosure, the WNT pathway agonist is present in the culture medium at a concentration within a range of 0.25-0.75 μM, 0.3-0.7 μM, 0.4-0.6 μM or 0.45-0.55 μM. In some embodiments of the present disclosure, the WNT pathway agonist is present in the culture medium at a concentration of 0.5 μM. In some embodiments of the present disclosure, the WNT pathway agonist is CHIR98014. In some embodiments of the present disclosure, the WNT pathway agonist is CHIR98014, which is present in the culture medium at a concentration within a range of 0.25-0.75 μM, 0.3-0.7 μM, 0.4-0.6 μM or 0.45-0.55 μM. In some embodiments of the present disclosure, the WNT pathway agonist is CHIR98014, which is present in the culture medium at a concentration of 0.5 μM.

Antagonists of the BET pathway include agents, molecules, compounds, or substances capable of inhibiting (downregulating) BET proteins (“bromodomain and extra-terminal motif” proteins), which comprise a bromodomain(s). Bromodomains are protein interaction modules that selectively recognize e-N-acetylated lysine residues (Kac). The human BET family (BRD2, BRD3, BRD4 and BRDT), which all contain two conserved bromodomains per target, plays a key role regulating transcription of growth stimulating genes.

In some embodiments of the present disclosure, the BET pathway antagonist is a triazolo-diazepine compound, non-limiting examples of which include (+)-JQ1 (described in, e.g., Filippakopoulos et al., 2010), TEN-010, OTX015, as well as structurally-related compounds. In some embodiments of the present disclosure, the triazolo-diazepine compound is (+)-JQ1. The person skilled in the art will appreciate, that various other BET pathway antagonists have been described. In some embodiments of the present disclosure, the BET pathway antagonist is selected from the group consisting of (+)-JQ1, TEN-010, OTX015, I-BET762, I-BET151, BAY1238097, ABBV-744, ABBV-075, iBET-BD1, iBET-BD2, SJ432, RVX-208, MS417, AZD5153, and combinations thereof. In some embodiments of the present disclosure, the BET pathway antagonist is present in the culture medium at a concentration within a range of 25-75 nM, 30-70 nM, 40-60 nM or 45-55 nM. In some embodiments of the present disclosure, the BET pathway antagonist is present in the culture medium at a concentration of 50 nM. In some embodiments of the present disclosure, the BET pathway antagonist is (+)-JQ1, which is present in the culture medium at a concentration within a range of 25-75 nM, 30-70 nM, 40-60 nM or 45-55 nM. In some embodiments of the present disclosure, the BET pathway antagonist is (+)-JQ1 at a concentration of 50 nM.

Agonists of the PDGF (platelet-derived growth factor) pathway include agents, molecules, compounds, or substances capable of stimulating (upregulating) a signaling pathway initiated by binding of a PDGF (e.g., PDGF-AA, PDGF-AB or PDGF-BB) to its receptor. In some embodiments of the present disclosure, the PDGF pathway agonist is PDGF-BB. In some embodiments of the present disclosure, the PDGF agonist is 740Y-P. In some embodiments of the present disclosure, the PDGF pathway agonist is PDGF-BB, which is present in the culture medium at a concentration within a range of 7.5-12.5 ng/ml, 8.0-12.0 ng/ml, 9.0-11.0 ng/ml, 9.5-10.5 ng/ml or 10 ng/ml.

Agonists of the IGF1 (insulin-like growth factor 1) pathway include agents, molecules, compounds, or substances capable of stimulating (upregulating) a signaling pathway initiated by binding of IGF1 to its receptor. In some embodiments of the present disclosure, the IGF1 pathway agonist is IGF1. Other examples of IGF1 pathway agonists include agonistic peptides, such as IGF1 30-41 peptide and IGF1 24-41 peptide. In some embodiments of the present disclosure, the IGF1 pathway agonist is IGF1, which is present in the culture medium at a concentration within a range of 15-25 ng/ml, 16.5-23.5 ng/ml, 17.5-22.0 ng/ml, 19.0-21.0 ng/ml or 20 ng/ml.

Agonists of the FGF-beta (fibroblast growth factor-beta) pathway include agents, molecules, compounds, or substances capable of stimulating (upregulating) a signaling pathway initiated by binding of FGF-beta to its receptor. In some embodiments of the present disclosure, the FGF-beta pathway agonist is FGF-beta. In some embodiments of the present disclosure, the FGF-beta pathway agonist is FGF-beta, which is present in the culture medium at a concentration within a range of 7.5-12.5 ng/ml, 8.0-12.0 ng/ml, 9.0-11.0 ng/ml, 9.5-10.5 ng/ml or 10 ng/ml.

In some embodiments of the present disclosure, the culture medium comprises a base medium. In some embodiments of the present disclosure, the base medium is DMEM/F12 medium, although other media of similar components to DMEM/F12 are also suitable for use as base medium. In some embodiments of the present disclosure, the base medium comprises serum. In some embodiments of the present disclosure, the serum is selected from fetal bovine serum and human serum. In some embodiments of the present disclosure, the base medium is DMEM/F12 media with 10% fetal bovine serum. In some embodiments of the present disclosure, the base medium does not comprise serum. In some embodiments of the present disclosure, the base medium is xeno-free.

In some embodiments of the present disclosure, the L-glutamine supplement is an L-alanine-L-glutamine dipeptide.

In combination with the defined culture media described above, the methods of generating iMSCs utilize standard culture conditions established in the art for cell culture. For example, cells can be cultured at 37° C. and under 20% O₂ and 5% CO₂ conditions.

In some embodiments of the present disclosure, the cells cultured in the above described differentiation medium are cultured for a sufficient time such that at least 90% (more preferably at least 91%, 92%, 93%, 94%, 95%, 96%, 97%, 98% or 99%) of the cells within the cell population express at least one, and preferably multiple (e.g., at least two, three, four or five) mesenchymal cell surface markers. Non-limiting examples of mesenchymal cell surface markers include CD73, CD90, CD105, CD29, CD44, and combinations thereof. In some embodiments of the present disclosure, the cells cultured in the above described differentiation medium are cultured for a sufficient time such that less than 5% (more preferably less than 4%, 3%, 2%, 1%, or 0.5%) of the cells within the cell population express one or more markers selected from the group consisting of TRA-1-60, CD34, CD45, HLA-DR, and combinations thereof.

In some embodiments of the present disclosure, the mRNA construct can be detected in the MSCs at least 1 day, such as at least 2 days, such as at least 3 days, such as at least 5 days, such as at least 7 days, such as at least 8 days, such as at least 10 days, such as at least 12 days, such as at least 15 days, such as at least 20 days after transfection. In some embodiments of the present disclosure, the mRNA construct can be detected in the MSCs at least 1 day after transfection. In some embodiments of the present disclosure, the mRNA construct can be detected in the MSCs at least 2 days after transfection. In some embodiments of the present disclosure, the mRNA construct can be detected in the MSCs at least 3 days after transfection. In some embodiments of the present disclosure, the mRNA construct can be detected in the MSCs at least 4 days after transfection. In some embodiments of the present disclosure, the mRNA construct can be detected in the MSCs at least 5 days after transfection. In some embodiments of the present disclosure, the mRNA construct can be detected in the MSCs at least 6 days after transfection. In some embodiments of the present disclosure, the mRNA construct can be detected in the MSCs at least 7 days after transfection. In some embodiments of the present disclosure, the mRNA construct can be detected in the MSCs at least 8 days after transfection. In some embodiments of the present disclosure, the mRNA construct can be detected in the MSCs at least 9 days after transfection. In some embodiments of the present disclosure, the mRNA construct can be detected in the MSCs at least 10 days after transfection. In some embodiments of the present disclosure, the mRNA construct can be detected in the MSCs at least 11 days after transfection. In some embodiments of the present disclosure, the mRNA construct can be detected in the MSCs at least 12 days after transfection. In some embodiments of the present disclosure, the mRNA construct can be detected in the MSCs at least 13 days after transfection. In some embodiments of the present disclosure, the mRNA construct can be detected in the MSCs at least 14 days after transfection. In some embodiments of the present disclosure, the mRNA construct can be detected in the MSCs at least 15 days after transfection. In some embodiments of the present disclosure, the mRNA construct can be detected in the MSCs at least 16 days after transfection. In some embodiments of the present disclosure, the mRNA construct can be detected in the MSCs at least 17 days after transfection. In some embodiments of the present disclosure, the mRNA construct can be detected in the MSCs at least 18 days after transfection. In some embodiments of the present disclosure, the mRNA construct can be detected in the MSCs at least 19 days after transfection. In some embodiments of the present disclosure, the mRNA construct can be detected in the MSCs at least 20 days after transfection.

In some embodiments of the present disclosure, after 1 day at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct per 1×10⁶ mesenchymal stem cells can be detected. In some embodiments of the present disclosure, after 2 days at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct per 1×10⁶ mesenchymal stem cells can be detected. In some embodiments of the present disclosure, after 3 days at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA per 1×10⁶ mesenchymal stem cells can be detected. In some embodiments of the present disclosure, after 5 days at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct per 1×10⁶ mesenchymal stem cells can be detected. In some embodiments of the present disclosure, after 7 days at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct per 1×10⁶ mesenchymal stem cells can be detected. In some embodiments of the present disclosure, after 8 days at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct per 1×10⁶ mesenchymal stem cells can be detected. In some embodiments of the present disclosure, after 10 days at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct per 1×10⁶ mesenchymal stem cells can be detected. In some embodiments of the present disclosure, after 12 days at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct per 1×10⁶ mesenchymal stem cells can be detected.

The person skilled in the art will appreciate that several methods can be used to detect the amount of mRNA molecules in a cell. In some embodiments of the present disclosure, the method of detecting the amount of mRNA molecules in the mesenchymal stem cells is selected from the group consisting of RNA sequencing, northern analysis, nuclease protection assays, In-situ hybridization and RT-PCR.

In some embodiments of the present disclosure, the protein encoded by said mRNA construct can be detected in the supernatant at least 1 day, such as at least 2 days, such as at least 3 days, such as at least 5 days, such as at least 7 days, such as at least 8 days, such as at least 10 days, such as at least 12 days, such as at least 15 days, such as at least 20 days after transfection. As used herein, the term “supernatant” refers to the non-cellular material produced following the in vitro culturing of mesenchymal precursor cells, and/or progeny cells thereof, in a suitable medium, preferably liquid medium. Typically, the supernatant is produced by culturing the cells in the medium under suitable conditions and time, followed by removing the cellular material by a process such as centrifugation.

In some embodiments of the present disclosure, 1 day after transfection 1000 cells express at least 10 pg, such as at least 100 pg, such as at least 1 ng, such as at least 1.25 ng, such as at least 1.5 ng, such as at least 1.75 ng, such as at least 2 ng protein, wherein the protein encoded by said mRNA construct can be detected in the supernatant. In some embodiments of the present disclosure, 2 days after transfection 1000 cells express at least 10 pg, such as at least 100 pg, such as at least 1 ng, such as at least 1.25 ng, such as at least 1.5 ng, such as at least 1.75 ng, such as at least 2 ng protein, wherein the protein encoded by said mRNA construct can be detected in the supernatant. In some embodiments of the present disclosure, 3 days after transfection 1000 cells express at least 10 pg, such as at least 100 pg, such as at least 1 ng, such as at least 1.25 ng, such as at least 1.5 ng, such as at least 1.75 ng, such as at least 2 ng protein, wherein the protein encoded by said mRNA construct can be detected in the supernatant. In some embodiments of the present disclosure, 5 days after transfection 1000 cells express at least 10 pg, such as at least 100 pg, such as at least 1 ng, such as at least 1.25 ng, such as at least 1.5 ng, such as at least 1.75 ng, such as at least 2 ng protein, wherein the protein encoded by said mRNA construct can be detected in the supernatant. In some embodiments of the present disclosure, 7 days after transfection 1000 cells express at least 10 pg, such as at least 100 pg, such as at least 1 ng, such as at least 1.25 ng, such as at least 1.5 ng, such as at least 1.75 ng, such as at least 2 ng protein, wherein the protein encoded by said mRNA construct can be detected in the supernatant.

In some embodiments of the present disclosure, 8 days after transfection 1000 cells express at least 10 pg, such as at least 100 pg, such as at least 1 ng, such as at least 1.25 ng, such as at least 1.5 ng, such as at least 1.75 ng, such as at least 2 ng protein, wherein the protein encoded by said mRNA construct can be detected in the supernatant.

In some embodiments of the present disclosure, 10 days after transfection 1000 cells express at least 10 pg, such as at least 100 pg, such as at least 1 ng, such as at least 1.25 ng, such as at least 1.5 ng, such as at least 1.75 ng, such as at least 2 ng protein. In some embodiments of the present disclosure, 12 days after transfection 1000 cells express at least 10 pg, such as at least 100 pg, such as at least 1 ng, such as at least 1.25 ng, such as at least 1.5 ng, such as at least 1.75 ng, such as at least 2 ng protein.

In another aspect, the present disclosure relates to an engineered MSC comprising an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 25. In another aspect, the present disclosure relates to an engineered MSC comprising an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 26. In another aspect, the present disclosure relates to an engineered MSC comprising an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 25. In another aspect, the present disclosure relates to an engineered MSC comprising an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 26.

In some embodiments of the present disclosure, the MSC is generated by a method as described herein.

The MSCs generated by the method as described in this section can also be called iMSCs. The MSCs generated by the methods described in this section, which include the use of human induced pluripotent stem cells (hiPSCs), can be referred to as iMSCs. Thus, the term “iMSCs” specifically denotes mesenchymal stem cells obtained through directed differentiation of hiPSCs using the defined protocols described in this section. In certain embodiments, the present disclosure leverages induced mesenchymal stem cells (iMSCs) as a delivery platform for mRNA constructs. iMSCs can offer a range of advantages over conventional mRNA delivery methods. Compared to lipid nanoparticles (LNPs), iMSCs can exhibit substantially lower systemic toxicity and reduced immunogenicity, making them particularly suitable for chronic or repeated dosing regimens. Unlike viral vectors, iMSCs may not pose an inherent risk of insertional mutagenesis or oncogenesis, and they can circumvent size limitations associated with viral capsids, thereby enabling the delivery of large or complex nucleic acid payloads. In contrast to extracellular vesicles (EVs) and viral vectors, iMSCs may demonstrate superior efficiency in cargo loading and sustained payload delivery. From a therapeutic perspective, iMSCs may provide additional benefits over other cell types. Mesenchymal stem cells (MSCs) may exert beneficial effects in conditions such as osteoarthritis (OA), yet traditional sources such as bone marrow-derived MSCs display limited regenerative potential and poor scalability. Similarly, fetal MSCs suffer from restricted scalability and donor availability. iMSCs may overcome these limitations by offering an expandable and renewable source of therapeutic cells that are not patient-specific and can be manufactured at scale for allogeneic use, thereby enabling off-the-shelf therapeutic applications.

Methods of generating induced mesenchymal stem cells are also described in US Patent Publication US 2023/0257714 and PCT Publication WO 2023/148556, the entire contents of each of which are incorporated herein by reference.

### LNPs

Lipid nanoparticles (LNPs) are well-established carriers for the delivery of nucleic acids, particularly mRNA, and have been successfully employed in clinical settings, including vaccine and gene therapy applications. LNPs can consist of an ionizable lipid, helper lipid(s), cholesterol, and/or PEGylated lipids, which self-assemble into nanoparticles capable of encapsulating and protecting nucleic acid cargo. The LNP structure can facilitate cellular uptake and endosomal escape, allowing the release of functional mRNA into the cytoplasm of target cells. The preparation of LNPs comprising mRNA constructs is considered routine in the art, and the skilled person would be able to incorporate a desired mRNA sequence into an LNP formulation using known techniques such as microfluidic mixing or ethanol injection.

In some embodiments, the present disclosure provides a lipid nanoparticle (LNP) formulation comprising an mRNA construct encoding TGFβ3 of SEQ ID NO: 29 or a functional homologue thereof, said homologue sharing at least 70%, such as at least 80%, for example at least 90%, such as at least 95% sequence identity with SEQ ID NO: 29, for use in the treatment of arthritis.

In some embodiments, the present disclosure provides a lipid nanoparticle (LNP) formulation comprising an mRNA construct encoding IGF1 of SEQ ID NO: 23 or a functional homologue thereof, said homologue sharing at least 70%, such as at least 80%, for example at least 90%, such as at least 95% sequence identity with SEQ ID NO: 23, for use in the treatment of arthritis.

In some embodiments, the present disclosure provides a lipid nanoparticle (LNP) formulation comprising an mRNA construct encoding BMP6 of SEQ ID NO: 17 or a functional homologue thereof, said homologue sharing at least 70%, such as at least 80%, for example at least 90%, such as at least 95% sequence identity with SEQ ID NO: 17, for use in the treatment of arthritis.

In some embodiments, the present disclosure provides a lipid nanoparticle (LNP) formulation comprising an mRNA construct encoding IL-10 of SEQ ID NO: 5 or a functional homologue thereof, said homologue sharing at least 70%, such as at least 80%, for example at least 90%, such as at least 95% sequence identity with SEQ ID NO: 5, for use in the treatment of arthritis.

In some embodiments, the present disclosure provides a lipid nanoparticle (LNP) formulation comprising an mRNA construct encoding IL1RN of SEQ ID NO: 11 or a functional homologue thereof, said homologue sharing at least 70%, such as at least 80%, for example at least 90%, such as at least 95% sequence identity with SEQ ID NO: 11, for use in the treatment of arthritis.

In some embodiments, the present disclosure provides a lipid nanoparticle (LNP) formulation comprising an mRNA construct encoding WNT3a of SEQ ID NO: 35 or a functional homologue thereof, said homologue sharing at least 70%, such as at least 80%, for example at least 90%, such as at least 95% sequence identity with SEQ ID NO: 35, for use in the treatment of arthritis.

In some embodiments, the mRNA is a chemically modified mRNA, comprising one or more modified nucleotides selected from pseudouridine, 5-methylcytidine, and N1-methylpseudouridine.

In another embodiment, the mRNA construct is chemically unmodified, and comprises only naturally occurring nucleotides. In a further embodiment, the mRNA construct does not contain chemically modified bases.

In some embodiments, the LNP comprises one or more lipids selected from ionizable lipids, helper lipids, cholesterol, and PEGylated lipids. Non-limiting examples include DLin-MC3-DMA, DSPC, cholesterol, and DMG-PEG2000.

In some embodiments, the LNP formulation is intended for intra-articular administration.

The amount of mRNA encapsulated in each nanoparticle may vary depending on formulation conditions and intended dose. In some embodiments, the LNPs may be formulated such that each nanoparticle comprises between 10 and 10,000 mRNA molecules, such as between 50 and 5,000 molecules, for example between 100 and 2,000 molecules. In further embodiments, the total mRNA dose per intra-articular administration may range from 10 ng to 1 mg, such as from 100 ng to 100 μg, for example from 1 μg to 10 μg, depending on disease severity, joint size, and patient response.

The skilled person will understand that various known formulation techniques may be used to achieve the desired encapsulation efficiency and particle size, including control of lipid-to-RNA ratio, flow rates, and buffer conditions during mixing.

mRNA Construct

In some embodiments of the present disclosure, at least some residues, and in some cases all residues of the mRNA construct, are chemically unmodified. In some embodiments of the present disclosure, at least some residues, and in some cases all residues of the open-reading frame of the mRNA construct, are chemically unmodified. As used herein, a “chemically unmodified” residue within an mRNA construct refers to a nucleobase, nucleoside or nucleotide within the construct that does not contain a chemical modification, such as a methylation. As used herein “nucleoside” refers to a compound containing a sugar molecule (e.g., a pentose or ribose) or derivative thereof in combination with an organic base (e.g., a purine or pyrimidine) or a derivative thereof (also referred to herein as “nucleobase”). As used herein, “nucleotide” refers to a nucleoside including a phosphate group. A “chemically unmodified” residue within an mRNA construct differs from a “chemically modified” residue within an mRNA construct in that the latter comprises a chemical modification of the nucleobase, nucleoside or nucleotide.

For example, a common modification in an mRNA construct comprises use of a chemically modified uridine nucleoside, such as N1-Methylpseudouridine (m1ψ), in place of the chemically unmodified uridine residues throughout the mRNA construct, referred to herein as a “uridine-modified” mRNA construct. In contrast, a “uridine-unmodified” mRNA construct of the disclosure uses chemically unmodified uridines throughout the construct. In some embodiments of the present disclosure, an mRNA construct of the disclosure is a uridine-unmodified mRNA construct, wherein all uridine nucleosides in the mRNA construct are chemically unmodified. For example, a chemically unmodified mRNA construct of the disclosure can lack N1-Methylpseudouridine (m1ψ) at one or more (e.g., at all) uridine positions within the sequence of the mRNA construct.

Another common mRNA modification comprises use of a chemically modified cytidine nucleoside, such as 5-methyl cytidine, in place of the chemically unmodified cytidine residues throughout the mRNA construct, referred to herein as a “cytidine-modified” mRNA construct. In contrast, a “cytidine-unmodified” mRNA construct of the disclosure uses chemically unmodified cytidines throughout the construct. In some embodiments of the present disclosure, an mRNA construct of the disclosure is a cytidine-unmodified mRNA construct, wherein all cytidine nucleosides in the mRNA construct are chemically unmodified. For example, a chemically unmodified mRNA construct of the disclosure can lack 5-methyl cytidine at one or more (e.g., at all) cytidine positions within the sequence of the mRNA construct.

In some embodiments of the present disclosure, an mRNA construct of the disclosure is a fully unmodified mRNA construct, wherein all nucleosides in the mRNA construct are chemically unmodified. In some embodiments of the present disclosure, an mRNA construct of the disclosure comprises a fully unmodified open-reading frame, wherein all nucleosides in the open-reading frame are chemically unmodified. The person skilled in the art will appreciate that the mRNA will consist of the nucleosides U, A, C and G before introducing the mRNA into the cell. Furthermore, the skilled person in the art will appreciate, that the mRNA can be modified by the cellular machinery after the mRNA is introduced into the cell.

An mRNA construct of the disclosure comprises an open reading frame (ORF) that encodes a protein. In some embodiments of the present disclosure, the mRNA construct encodes a secreted protein, such as TGFβ3.

In addition to the ORF, the mRNA construct typically comprises additional regulatory elements such as a 5′ untranslated region (5′-UTR), a 3′ untranslated region (3′-UTR) and/or a poly-A tail. Such additional mRNA elements are well established and available in the art. In some embodiments of the present disclosure, the mRNA construct comprises a 5′ untranslated region (UTR). In some embodiments of the present disclosure, the mRNA construct comprises a 3′ untranslated region (UTR). In some embodiments of the present disclosure, the mRNA construct comprises a 5′ cap. In some embodiments of the present disclosure, the mRNA construct comprises a poly-A tail. In some embodiments of the present disclosure, the UTR is a α-globin UTR. In some embodiments of the present disclosure, the UTR is a β-globin UTR. In some embodiments of the present disclosure, the UTR is customized for the mesenchymal stem cell. In some embodiments of the present disclosure, the UTR is customized for the disease microenvironment.

The mRNA construct comprises a suitable number of bases to encompass the ORF and additional elements.

In some embodiments of the present disclosure, the mRNA construct comprises a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG.

An mRNA construct can comprise a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′, 3′-dideoxycytosine, 2′,3′-dideoxyguanosine, and 2′,3′-dideoxythymine.

In some embodiments of the present disclosure, the mRNA construct does not comprise modified nucleosides in the ORF.

An mRNA construct can comprise a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments of the present disclosure, a polyA sequence may affect the nuclear export, translation, and/or stability of an mRNA. An mRNA construct can comprise a microRNA binding site. The sequences of numerous microRNA binding sites are well known in the art.

In some embodiments of the present disclosure, an mRNA construct may be codon optimized. For example, an mRNA construct can be codon optimized to enhance expression of the unmodified mRNA. Moreover, additional codon optimization approaches are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, Calif.) and/or proprietary methods. In some embodiments of the present disclosure, the mRNA sequence is optimized using optimization algorithms, e.g., to optimize expression in mammalian cells or enhance mRNA stability.

In some embodiments of the present disclosure, the mRNA construct is codon optimized
a. to enhance the expression in a subject; or
b. to optimize the uridine content; or
c. to increase the stability in the MSCs.

For example, constructs can be generated after basic codon optimization using IDT codon optimization for expression in humans. This codon optimization algorithm relies on rebalancing codon usage, decreasing sequence complexity and avoiding rare codons. It is mainly used to minimize secondary structures and reduce complexity. In some embodiments, the IDT codon optimization is performed as essentially described in https://eu.idtdna.com/pages/education/decoded/article/idt-codon-optimization-tool-makes-synthetic-gene-design-easy (25 Jun. 2024).

In some embodiments, the mRNA construct is codon optimized to reduce the uridine content.

An mRNA construct of the disclosure may be produced by standard means available in the art, including but not limited to in vitro transcription (IVT) and synthetic methods. Enzymatic (IVT), solid-phase, liquid-phase, combined synthetic methods, small region synthesis, and ligation methods may be utilized. In some embodiments of the present disclosure, mRNAs are made using IVT enzymatic synthesis methods. Methods of making polynucleotides by IVT are well known in the art. In some embodiments of the present disclosure, the method to synthesize the mRNA construct is selected from the group of Enzymatic (IVT) methods, solid-phase methods, liquid-phase methods, combined synthetic methods, small region synthesis, and ligation methods. In a preferred embodiment, the mRNA construct is synthesized via in vitro transcription (IVT). In some embodiments of the present disclosure, a T7 polymerase is used for synthesizing the mRNA construct.

In some embodiments of the present disclosure, at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct, such as at least 7 μg mRNA construct, such as at least 10 μg mRNA construct, such as at least 100 μg mRNA construct is introduced into 1×10⁶ mesenchymal stem cells.

In some embodiments of the present disclosure, at most 1 mg mRNA construct, such as at most 100 μg mRNA construct, such as 50 μg mRNA construct, such as at most 25 μg mRNA construct, such as at most 20 μg mRNA construct, such as at most 10 μg mRNA construct is introduced into 1×10⁶ mesenchymal stem cells.

In some embodiments of the present disclosure, between 1 ng mRNA construct and 1 mg mRNA construct, such as between 10 ng mRNA construct and 100 μg mRNA construct, such as between 0.5 μg mRNA construct and 50 μg mRNA construct, such as between 1 μg mRNA construct and 25 μg mRNA construct, such as between 5 μg mRNA construct and 20 μg mRNA construct is introduced into 1×10⁶ mesenchymal stem cells.

In some embodiments of the present disclosure, 1×10⁶ MSCs comprise at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct, such as at least 7 μg mRNA construct, such as at least 10 μg mRNA construct, such as at least 100 μg mRNA construct.

In some embodiments of the present disclosure, 1×10⁶ mesenchymal stem cells comprise at most 1 mg mRNA construct, such as at most 100 μg mRNA construct, such as 50 μg mRNA construct, such as at most 25 μg mRNA construct, such as at most 20 μg mRNA construct, such as at most 10 μg mRNA construct.

In some embodiments of the present disclosure, 1×10⁶ mesenchymal stem cells comprise between 1 ng mRNA construct and 1 mg mRNA construct, such as between 10 ng mRNA construct and 100 μg mRNA construct, such as between 0.5 μg mRNA construct and 50 μg mRNA construct, such as between 1 μg mRNA construct and 25 μg mRNA construct, such as between 5 μg mRNA construct and 20 μg mRNA construct.

In some embodiments of the present disclosure, 1×10⁶ MSCs comprise at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct, such as at least 7 μg mRNA construct, such as at least 10 μg mRNA construct, such as at least 100 μg mRNA construct at the time of administration.

In some embodiments of the present disclosure, 1×10⁶ mesenchymal stem cells comprise at most 1 mg mRNA construct, such as at most 100 μg mRNA construct, such as 50 μg mRNA construct, such as at most 25 μg mRNA construct, such as at most 20 μg mRNA construct, such as at most 10 μg mRNA construct at the time of administration.

In some embodiments of the present disclosure, 1×10⁶ mesenchymal stem cells comprise between 1 ng mRNA construct and 1 mg mRNA construct, such as between 10 ng mRNA construct and 100 μg mRNA construct, such as between 0.5 μg mRNA construct and 50 μg mRNA construct, such as between 1 μg mRNA construct and 25 μg mRNA construct, such as between 5 μg mRNA construct and 20 μg mRNA construct. at the time of administration.

In some embodiments of the present disclosure, the method of detecting the amount of mRNA molecules in the mesenchymal stem cells is selected from the group consisting of: RNA sequencing, northern analysis, nuclease protection assays, In-situ hybridization, labelled mRNAs, ddPCR and RT-PCR.

A number of approaches can be used to enhance expression of uridine-unmodified mRNA constructs when used either alone or, more preferably, in combination, as compared to uridine-modified mRNA constructs. These approaches include optimization of certain codons within the unmodified mRNA construct, purification of the unmodified mRNA preparation prior to introduction into the MSCs and introduction of the unmodified mRNA construct into the MSCs by a means that utilizes a non-endosomal pathway.

In some embodiments of the present disclosure, the mRNA construct is codon optimized prior to introduction into the MSCs, e.g., to enhance expression of the encoded secreted protein. In some embodiments of the present disclosure, the mRNA construct is engineered to reduce overall uridine content. Algorithms available in the art can be used to reduce uridine content in the mRNA construct. For example, the genescript algorithm can be used as a starting template for reducing uridine. This algorithm, described as “GenSmart™ Codon Optimization” uses an algorithm based on the “Population Immune Algorithm”, which takes advantage of both population genetics and immunology theories. In this approach, more than 200 factors involved in gene expression, including GC content, codon usage and content index, RNase splicing sites, and cis-acting mRNA destabilizing motifs, are screened and validated. Instead of applying a single-factor simulation to computing, a multifactor approach is employed to ensure that all key factors in a certain target gene sequence carry weight. As a result, each gene optimization is fully customized to maximize the likelihood of obtaining a functional and active protein. Additionally, algorithm-optimized sequences can be further processed by manually reducing their uridine content using codon substitution until the lowest level possible is obtained without generating aberrant structure complexities or secondary structures.

In some embodiments of the present disclosure, the codons of the coding sequence have been selected to reduce the uridine content by a method, wherein the method comprises following steps:
a. Codon optimization;
b. Reduction of uridine content by selecting uridine low or uridine free codons.

In some embodiments of the present disclosure, the codons are optimized to rebalance codon usage, decrease sequence complexity, avoid rare codons, minimize secondary structures and/or reduce complexity. In some embodiments of the present disclosure, the IDT codon optimization tool is used for codon optimization. In some embodiments of the present disclosure, the “GenSmart™ Codon Optimization tool is used to reduce the uridine content. In some embodiments of the present disclosure, the construct manufacturing complexity test is used to indicate foreseeable manufacturing problems. Therefore, in some embodiments of the present disclosure, the codons of the coding sequence have been selected to reduce the uridine content by a method, wherein the method comprises following steps:
a) Codon optimization;
b) Reduction of uridine content by selecting uridine low or uridine free codons;
c) Adjusting the codon to circumvent manufacturing problems.

Examples for manufacturing problems are hairpin structures or an unfavorable GC content. In some embodiments of the present disclosure, the absolute uridine content of the mRNA construct in the mRNA composition is reduced by at least 0.5%, such as at least 1%, such as at least 2.5%, such as at least 5%, such as at least 10%, such as at least 15% compared to the uridine content of the naturally occurring mRNA. In some embodiments of the present disclosure, the absolute uridine content of the mRNA construct in the mRNA composition is reduced by at most 15%, such as at most 10%, such as at most 5% compared to the uridine content of the naturally occurring mRNA. In some embodiments of the present disclosure, the absolute uridine content of the mRNA construct in the mRNA composition is reduced by between 0.5% and 15%, such as between 1% and 15%, such as between 2.5% and 15%, such as between 5% and 15%, such as by between 10% and 15% compared to the uridine content of the naturally occurring mRNA. Thus, in some embodiments of the present disclosure, the mRNA construct has been designed to reduce the uridine content.

In some embodiments of the present disclosure, the uridine content in the mRNA construct expressing IL-10 of SEQ ID NO: 6 or a functional homologue of said IL-10 sharing at least 70%, such as at least 80%, for example at least 90%, such as at least 95% sequence identity therewith is reduced to 19.2% while the uridine content of the naturally occurring mRNA of SEQ ID NO: 4 is 21.4%.

In some embodiments of the present disclosure, the uridine content in the mRNA construct expressing IL1RN of SEQ ID NO: 12 or a functional homologue of said ILRN1 sharing at least 70%, such as at least 80%, for example at least 90%, such as at least 95% sequence identity therewith is reduced to 19.7% while the uridine content of the naturally occurring mRNA of SEQ ID NO: 10 is 22.1%.

In some embodiments of the present disclosure, the uridine content in the mRNA construct expressing BMP6 of SEQ ID NO: 18 or a functional homologue of said BMP6 sharing at least 70%, such as at least 80%, for example at least 90%, such as at least 95% sequence identity therewith is reduced to 17.3% while the uridine content of the naturally occurring mRNA of SEQ ID NO: 16 is 18.2%.

In some embodiments of the present disclosure, the uridine content in the mRNA construct expressing IGF1 of SEQ ID NO: 24 or a functional homologue of said IGF1 sharing at least 70%, such as at least 80%, for example at least 90%, such as at least 95% sequence identity therewith is reduced to 19.3% while the uridine content of the naturally occurring mRNA of SEQ ID NO: 22 is 22.3%.

In some embodiments of the present disclosure, the uridine content in the mRNA construct expressing TGFB of SEQ ID NO: 30 or a functional homologue of said TGFB sharing at least 70%, such as at least 80%, for example at least 90%, such as at least 95% sequence identity therewith is reduced to 17.1% while the uridine content of the naturally occurring mRNA of SEQ ID NO: 28 is 20%.

In some embodiments of the present disclosure, the uridine content in the mRNA construct expressing WNT3a of SEQ ID NO: 36 or a functional homologue of said WNT3a sharing at least 70%, such as at least 80%, for example at least 90%, such as at least 95% sequence identity therewith is changed to 17.9% while the uridine content of the naturally occurring mRNA of SEQ ID NO: 34 is 16.5%.

In some embodiments of the present disclosure, the mRNA construct is purified prior to introducing the mRNA preparation into the MSCs, for example to remove double-stranded RNA (dsRNA) from the preparation. In some embodiments of the present disclosure, the mRNA preparation is subjected to at least one chromatography procedure prior to introduction into MSCs, and may be subjected to additional rounds of purification as needed. Methods for removing dsRNA using chromatographic procedures are well-established in the art. In some embodiments of the present disclosure, dsRNA is removed from the mRNA construct preparation using cellulose purification. Alternatively, other forms of purification that remove dsRNA or other impurities can be used (e.g., HPLC, silica based, polyA affinity annealing, etc). Assays known in the art, such as the Dotblot assay, can be used to evaluate the amount of dsRNA in an mRNA preparation, e.g., before and after purification of the preparation on a cellulose column.

In some embodiments of the present disclosure, at most 10% of all RNA in the mRNA composition is double-stranded RNA, such as at most 8%, such as at most 6%, such as at most 5%, such as at most 3%, such as at most 1%. In some embodiments of the present disclosure, between 0% and 10% of all RNA in the mRNA composition is double-stranded RNA, such as between 0% and 8%, such as between 0% and 6%, such as between 0% and 4%. Thus, in some embodiments of the present disclosure, the MSCs comprise a substantially single stranded composition of a mRNA construct introduced into the cell by a non-endosomal pathway of delivery, the mRNA construct comprising a coding sequence, wherein all nucleosides within the mRNA construct are chemically unmodified.

In some embodiments of the present disclosure, the method to remove dsRNA of the mRNA composition is selected from the group consisting of cellulose chromatography, High-performance liquid chromatography, silica based chromatography and polyA affinity annealing. In a preferred embodiment of the present disclosure, the dsRNA is removed by cellulose chromatography. In some embodiments of the present disclosure, the mRNA composition is purified at least once, such as at least twice, such as at least three times. In some embodiments of the present disclosure, the method to detect the amount of dsRNA in the mRNA composition is selected from the group consisting of Dot blot, lateral flow immunoassay, ELISA and dsRNA-sequencing. In a preferred embodiment of the present disclosure, the amount of dsRNA in the mRNA composition is determined via dot blot.

In some embodiments of the present disclosure, the mRNA construct is introduced into MSCs by means that utilize a non-endosomal pathway or approach of delivery. Endosomal pathways are a subset of endocytic pathways for internalizing molecules from the plasma membrane in which the internalized cargo is delivered into endosomes, where they are sorted for onward transport to distinct cellular destinations. Most chemically-based transfection reagents that are used to deliver mRNA constructs into cells, including liposomes and lipid nanoparticles (LNPs), utilize an endosomal pathway of delivery, resulting in the mRNA constructs being exposed to Toll-like receptors (TLRs). While not intending to be limited by mechanism, it has been found that avoiding exposure to these TLRs enables stronger tolerance and/or subsequent translation of mRNA constructs in MSCs.

Accordingly, the methods of the disclosure utilize a non-endosomal pathway or approach of delivery (i.e., the means of delivery does not deliver the mRNA construct into endosomes). In some embodiments of the present disclosure, the non-endosomal means of delivery utilizes a non-endocytic approach, such as a physical delivery method. A non-limiting example of such a method is electroporation, which creates temporary holes in the cell membrane and therefore avoids detection from TLRs situated in the intracellular vesicles. Use of electroporation to introduce nucleic acids into cells is well-established in the art. Electroporation can be performed using a commercially available device (e.g., Nucleofector 2b; Lonza) according to manufacturer's guidance. Other methods are known in the art that allow for a similar “perforation”-mediated delivery, non-limiting examples of which include biolistic particle delivery (e.g., using a gene gun) and ultrasonic nebulization (also referred to as sonification). Accordingly, in some embodiments of the present disclosure, the mRNA construct is delivered into cells by means selected from the group consisting of electroporation, biolistic particle delivery and ultrasonic nebulization.

In some embodiments of the present disclosure, the non-endosomal pathway of delivery is selected from the group consisting of Microinjection, Streptolysin-O permeabilization, permeabilization using anionic peptides and electroporation. In a preferred embodiment, the non-endosomal pathway of delivery is electroporation. Another example known in the art of a non-endosomal, physical delivery method for delivery of nucleic acids into cells is buffer-mediated delivery of naked mRNA, such as using a citrate solution. Accordingly, in some embodiments of the present disclosure, the mRNA construct is delivered into cells by buffer-mediated delivery of naked mRNA into the MSCs.

In some embodiments of the present disclosure, the MSCs comprise an mRNA composition comprising naked mRNA.

In other embodiments, the mRNA construct comprises chemically modified nucleotides. Thus, in some embodiments of the present disclosure, the mRNA construct comprises at least one modified nucleotide base. In some embodiments of the present disclosure, at least one uridine is replaced with a modified nucleotide base, such as Pseudouridine, 5-methoxyuridine, 2-thiouridine or N1-methyl-pseudouridine. In some embodiments of the present disclosure, at least one cytosine is replaced with a modified nucleotide base, such as 5-methylcytosine, 2′-O-methylcytosine, or N4-acetylcytidine.

In yet other embodiments, modifications may include adenosine analogues, such as N6-methyladenosine (m⁶A) or N6,2′-O-dimethyladenosine (m⁶Am), or guanosine analogues, such as 7-methylguanosine (m⁷G).

Expression and use of unmodified mRNA constructs in MSCs is also described in PCT Publication WO 2025/078604, the entire contents of which is incorporated herein by reference.

### Arthritis

The disclosure provides MSCs for the method of treatment of arthritis. Arthritis is a term often used to imply any disorder that affects joints. In some embodiments of the present disclosure, the MSCs can be used for the treatment of osteoarthritis and rheumatoid arthritis.

Thus, in some embodiments of the present disclosure, the arthritis is hemarthrosis, osteoarthritis, rheumatoid arthritis, Gout, septic arthritis, ankylosing spondylitis, Juvenile idiopathic arthritis, still's disease or psoriatic arthritis, preferably osteoarthritis or rheumatoid arthritis.

The MSCs can be administered to the joints of a subject. The effect can be achieved by a single-injection or by repeated injections. Thus, in some embodiments of the present disclosure, the MSCs are administered repeatedly to a subject. In some embodiments of the present disclosure, the MSCs are administered at least once, such as at least twice, such as at least three times, such as at least 5 times. In some embodiments of the present disclosure, the MSCs are administered at most 5 times, such as at most three times, such as at most twice, such as at most once. In some embodiments of the present disclosure, the MSCs are administered repeatedly to a subject with a 1-4-week interval. In some embodiments of the present disclosure, the MSCs are administered repeatedly to a subject, such as at least once a week, such as at least every two weeks, such as at least once a month, such as at least every two month, such as at least every six month, such as at least once a year, such as at least every second year, such as at least every 3 years.

In some embodiments of the present disclosure, the subject is a subject diagnosed with arthritis, such as osteoarthritis or rheumatoid arthritis. Thus, in some embodiments, the MSCs are administered only to subjects affected by the disease, such as arthritis and/or pain.

In some embodiments of the present disclosure, the MSCs are administered in combination with a treatment selected from the group consisting of acetaminophen, NSAID, glucosamine, chondroitin, opioid, steroid and lubricants, such as hyaluronic acid.

In some embodiments of the present disclosure, the administration of the MSCs leads to joint damage repair. In some embodiments of the present disclosure, the administration of the MSCs leads to restoration of joint functions. Joint damage repair can be achieved by for example increase of cartilage in the joints. Restoration of joint function can be achieved by for example differentiation of MSCs into chondrocytes. In some embodiments of the present disclosure, the administration of the MSCs leads to pain relief. The desired level of pain relief may be, for example, complete removal of the pain or a reduction in the severity of the pain.

The arthritic condition to be treated may include, for example, osteoarthritis, rheumatoid arthritis, psoriatic arthritis, juvenile idiopathic arthritis, gout, septic arthritis, Still's disease, or ankylosing spondylitis, among others. Treatment may be administered during early, moderate, or advanced stages of the disease and may be performed as a monotherapy or in combination with other pharmacological or biological agents. Such treatment may be achieved, for instance, by the administration of mesenchymal stem cells (MSCs) engineered to express pro-chondrogenic or anti-inflammatory factors.

### Pain

In some embodiments, the administration of the MSCs may result in restoration of joint function. Thus, in some embodiments, the administration of MSCs may result in the restoration of joint function across various anatomical sites. This may include the knee joint, shoulder joint, and hand joints. Additional examples may include the hip joint, elbow joint, ankle joint, wrist joint, and temporomandibular joint (TMJ). Restoration of joint function may manifest as improved range of motion, reduced pain, enhanced stability, and increased load-bearing capacity. This effect may be mediated by differentiation of the MSCs into chondrocytes and/or osteoblasts, or through paracrine secretion of factors that promote cartilage regeneration, synovial homeostasis, and suppression of inflammation. Restoration of joint function may include, for example, improved joint mobility, increased weight-bearing capacity, reduced joint stiffness, and normalization of gait or range of motion.

As used herein, the term “synovial homeostasis” comprises the maintenance of a physiologically stable and functional state within the synovial joint, particularly with respect to the composition, function, and regulation of the synovial membrane and synovial fluid. Synovial homeostasis may include, for example balanced production and turnover of synovial fluid components, such as hyaluronic acid and lubricin, regulation of pro-inflammatory and anti-inflammatory cytokines and/or preservation of the joint cartilage microenvironment.

In some embodiments, the administration of the MSCs may lead to alleviation of pain associated with joint disorders. Pain relief may arise from multiple biological mechanisms, including reduction of joint inflammation, inhibition of pain-mediating cytokines such as TNF-α and IL-1β, repair/regeneration of damaged neural and/opr musculoskeletal tissues, and modulation of peripheral nociceptive pathways. In some embodiments, the level of pain relief may comprise a substantial reduction in the severity of pain, such as a reduction in pain score by at least 30%, such as at least 50%, such as at least 75%. In some embodiments, the administration of the MSCs as described herein may lead to complete elimination of pain symptoms. The person skilled in the art, will appreciate that there are several ways to assess pain, such as reported by the subject or measured by standardized assessment tools. For example, standardized assessments tools may comprise the Visual Analog Scale (VAS), the Numerical Rating Scale (NRS), the Western Ontario and McMaster Universities Osteoarthritis Index (WOMAC), the McGill Pain Questionnaire (MPQ), the Brief Pain Inventory (BPI), and/or the Short Form-36 (SF-36) Health Survey, as well as directed neurological and musculoskeletal evaluation like selective nerve root block, medial branch block, provocative discography, facet joint injection and sacroiliac joint injection.

In the context of pain, the treatment may be directed toward various forms of pain including, but not limited to, nociceptive pain, neuropathic pain and central sensitization, like inflammatory pain, degenerative pain, as well as mixed or complex pain syndromes associated with musculoskeletal or joint-related conditions. The duration of pain could be acute or chronic.

For example, treatment of nociceptive pain may involve reducing mechanical or chemical stimuli that activate peripheral nociceptors; treatment of inflammatory pain may include downregulating pro-inflammatory cytokines or disrupting sensitization pathways; treatment of degenerative pain may comprise preserving or restoring structural integrity of joint components to reduce biomechanical stress; and treatment of neuropathic pain may involve modulation of neuronal excitability, neuroinflammation, or aberrant signaling in the somatosensory system.

Such treatment may be achieved, for instance, by the administration of mesenchymal stem cells (MSCs) capable of exerting anti-inflammatory, immunomodulatory, neuroprotective, or regenerative effects, either directly or via paracrine mechanisms. The MSCs may secrete factors such as IL-10, TGF-β3, WNT3a, BMP6, II1RN and IGF1. In some embodiments, the treatment may also reduce the need for systemic analgesics or enable recovery of pain-free mobility.

In some embodiments, the therapeutic effect may be sustained over an extended period following administration, for example at least one day, such as at least one week, such as at least one month, such as at least three months, such as at least six months. In some embodiments, repeated or sequential administrations of MSCs may be employed to achieve or maintain the desired therapeutic outcome, including long-term pain relief, sustained improvement of joint function, and durable tissue repair.

In some embodiments, the administration of the MSCs leads to nociceptive pain relief. The desired level of pain relief may be, for example, complete removal of the nociceptive pain or a reduction in the severity of the nociceptive pain.

In some embodiments, the administration of the MSCs leads to inflammatory pain relief. The desired level of pain relief may be, for example, complete removal of the inflammatory pain or a reduction in the severity of the inflammatory pain. In some embodiments, the administration of the MSCs leads to relief of pain, wherein the therapeutic effect is mediated, at least in part, by paracrine secretion of at least one of the factors of WNT3a, IL-10, IL1RN, BMP6, TGFβ3 or IGF1, preferably TGFβ3. The mechanism of action of the factor may involve a direct or indirect modulatory effect on neurons located in the dorsal root ganglion (DRG).

In some embodiments, at least one of the factors of WNT3a, IL-10, IL1RN, BMP6, TGFβ3 or IGF1, preferably WNT3a exerts its effect through retrograde axonal transport, wherein the signal is conveyed from the peripheral nerve terminals at the site of administration through the axon of the sensory neuron toward the cell body located in the DRG.

In further embodiments, the therapeutic modulation of DRG neurons by at least one of the factors of WNT3a, TGF-33, IL-10, IL1RN, BMP6, or IGF1, preferably WNT3a may involve downregulation of pro-nociceptive ion channels, suppression of inflammatory signaling cascades, or enhancement of neuroprotective or anti-nociceptive mediators.

In some embodiments, the administration of the MSCs leads to degenerative pain relief. The desired level of pain relief may be, for example, complete removal of the degenerative pain or a reduction in the severity of the degenerative pain.

In some embodiments, the administration of the MSCs leads to neuropathic pain relief. The desired level of pain relief may be, for example, complete removal of the neuropathic pain or a reduction in the severity of the neuropathic pain.

As used herein, the term “nociceptive pain” comprises pain that arises from the activation of nociceptors—specialized sensory receptors—by actual or potential tissue-damaging stimuli. Nociceptive pain may result from mechanical, thermal, or chemical stimuli and is typically associated with acute injury or pathological conditions involving tissue stress. It may be localized, transient, or persistent, and may include sensations such as sharp, aching, or throbbing pain originating from joints, muscles, or other somatic structures.

As used herein, the term “inflammatory pain” comprises pain that occurs as a consequence of the inflammatory response to tissue injury, infection, or chronic immune activation. Inflammatory pain may result from the release of pro-inflammatory mediators such as prostaglandins, bradykinin, interleukins, or tumor necrosis factor-alpha (TNF-α), which sensitize or directly activate nociceptors. This type of pain is commonly observed in conditions such as rheumatoid arthritis, synovitis, or other immune-mediated joint disorders, and may present as persistent, throbbing, or pressure-like discomfort often accompanied by swelling, warmth, and stiffness.

As used herein, the term “degenerative pain” comprises pain that arises due to the structural deterioration of joint components, including cartilage, subchondral bone, and interarticular tissues, typically as part of chronic degenerative diseases such as osteoarthritis. Degenerative pain may result from mechanical instability, joint space narrowing, formation of osteophytes, or biochemical alterations in the extracellular matrix. It may manifest as intermittent or chronic pain, worsened by weight-bearing or joint use, and may be associated with reduced mobility and joint function.

As used herein, the term “neuropathic pain” comprises pain that results from damage to or dysfunction of the nervous system, including peripheral or central neural pathways. Neuropathic pain may arise from direct nerve injury, neuroinflammation, or abnormal neural signaling and may be characterized by burning, tingling, electric shock-like sensations, or hypersensitivity to normally non-painful stimuli (allodynia). In the context of joint disorders, neuropathic pain may develop secondary to chronic inflammation, nerve compression, or joint degeneration. Neuropathic pain can occur in a wide range of medical conditions. These include diabetes mellitus, multiple sclerosis, multiple myeloma, thyroid dysfunction, facial nerve disorders, and following procedures such as amputation or spine surgery. In addition, infections such as herpes zoster (shingles) and HIV, as well as chemotherapy-induced nerve damage, can cause neuropathic pain.

As used herein, the term “chronic pain” refers to pain that persists for longer than three months. It may originate as nociceptive pain—resulting from tissue damage and initially presenting as acute pain—which transitions into a chronic condition if unresolved. Alternatively, chronic pain may arise from neuropathic mechanisms, involving nerve injury or dysfunction, or from central sensitization, where there is no apparent tissue or nerve damage but the central nervous system becomes hypersensitive.

As used herein, the term “acute pain” refers to pain that arises suddenly in response to a clearly defined cause, such as injury, surgery, or inflammation. It is typically short-lived, lasting less than three months, and serves a protective biological function by alerting the body to harm and prompting a behavioral response to minimize injury. Acute pain is usually nociceptive in nature and resolves as the underlying cause heals. If the pain persists beyond the expected healing period, it may evolve into chronic pain.

As used herein, the term “central sensitization”, also referred to as “nociplastic pain”, describes a condition in which the central nervous system (CNS) undergoes structural, functional, or chemical changes. These changes result in long-lasting hypersensitivity to pain stimuli, even in the absence of ongoing peripheral tissue damage or nociceptor input. Individuals with central sensitization may experience allodynia (pain due to a normally non-painful stimulus) or hyperalgesia (increased sensitivity to painful stimuli).

In some embodiments, the administration of the MSCs leads to pain relief, wherein the therapeutic effect is achieved through a sequential administration protocol involving distinct populations of engineered MSCs. In particular, a first administration may comprise MSCs transfected with an mRNA construct encoding WNT3a, and a second administration, delivered thereafter in a defined temporal sequence, may comprise MSCs transfected with an mRNA construct encoding TGF-β3 and/or WNT3a. The two populations of MSCs may be administered, for example, at separate time points spaced by at least one day, such as at least two days, at least five days, or at least one week.

In some embodiments, MSCs transfected with an mRNA construct encoding WNT3a may provide an anti-inflammatory effect, while the subsequent administration of MSCs transfected with an mRNA construct encoding TGF-β3 may enhance matrix synthesis, resolution of inflammation, and long-term tissue stabilization. Together, the combined and temporally spaced action of MSCs transfected with an mRNA construct encoding WNT3a and TGF-β3 may lead to a synergistic or complementary effect on pain reduction.

The pain relief achieved through this sequential administration may be reflected in decreased joint pain scores, reduced mechanical hypersensitivity, or improved patient-reported pain-related outcomes, and may be associated with modulation of nociceptive signaling at the level of the joint, dorsal root ganglia, or spinal cord.

### Administration

In some embodiments of the present disclosure, at least 1×10⁵, such as at least 5×10⁵, such as at least 1×10⁶, such as at least 5×10⁶, such as at least 1×10⁷, such as at least 5×10⁷ cells are administered to the subject. In some embodiments of the present disclosure, the cells suspended in a pharmaceutically acceptable solution. In some embodiments of the present disclosure, the suspended cells are administered to the subject in a volume of at least 0.05 ml, such as at least 0.5 ml, such as at least 1 ml, such as at least 2 ml. In some embodiments of the present disclosure, the suspended cells are administered to the subject in a volume of at most 6 ml, such as at most 5 ml, such as at most 4 ml, such as at most 3 ml, such as at most 2 ml. In some embodiments of the present disclosure, the suspended cells are administered to the subject in a volume of 2 ml.

In some embodiments, the MSCs are not differentiated to chondrocytes and/or osteoblasts at the time of administration to the subject. In some embodiments, the administered MSCs may retain a surface marker profile characteristic of undifferentiated mesenchymal stem cells, including, for example, expression of CD73, CD90, and CD105, and absence of hematopoietic or lineage-specific markers such as CD34, CD45, CD19, CD11b, or HLA-DR.

In further embodiments, the absence of differentiation may be confirmed by a lack of expression of differentiation-associated markers such as SOX9 or ACAN (chondrocyte lineage), and RUNX2 or osteocalcin (BGLAP) (osteocyte lineage), as determined by flow cytometry, RT-qPCR, or immunohistochemical analysis.

In some embodiments, the method may be optimized to support the preservation of the undifferentiated phenotype, for example by avoiding prolonged exposure to lineage-inducing factors or cryopreserving the MSCs at early passages.

In some embodiments, the MSCs are administered at most 1 minute, such as at most 10 minutes, such as at most 30 minutes, such as at most 1 hour, such as at most 2 hours, such as at most 3 hours, such as at most 4 hours, such as at most 5 hours, such as at most 10 hours, such as at most 12 hours, such as at most 15 hours, such as at most 24 hours, such as at most 36 hours, such as at most 48 hours after transfection. In some embodiments, the MSCs are cryopreserved at most 1 minute, such as at most 10 minutes, such as at most 30 minutes, such as at most 1 hour, such as at most 2 hours, such as at most 3 hours, such as at most 4 hours, such as at most 5 hours, such as at most 10 hours, such as at most 12 hours, such as at most 15 hours, such as at most 24 hours, such as at most 36 hours, such as at most 48 hours after transfection. In some embodiments, the transfected MSCs are administered at most 1 minute, such as at most 10 minutes, such as at most 30 minutes, such as at most 1 hour, such as at most 2 hours, such as at most 3 hours, such as at most 4 hours, such as at most 5 hours, such as at most 10 hours, such as at most 12 hours, such as at most 15 hours, such as at most 24 hours, such as at most 36 hours, such as at most 48 hours after thawing.

In some embodiments of the present disclosure, the MSCs are used in a method of treatment of arthritis and/or prevention of arthritis as described herein, wherein said MSCs are generated by a method as described herein.

In some embodiments of the present disclosure, the MSCs are for use for a method of treatment of arthritis and/or prevention of arthritis as described herein, wherein said MSCs are MSCs as described herein.

### Combinations

The disclosure provides MSCs for the method of treatment of arthritis. Thereby, the MSCs are transfected with mRNA encoding a chondrogenesis and/or chondrocyte protection factor, such as TGFβ3. The treatment can be combined with MSCs transfected with mRNA encoding another anti-inflammatory factor, such as IL1RN or IL-10 or a factor promoting regeneration of chondrocytes, such as WNT3a, IGF1 or BMP6.

Interleukins (ILs) are a group of cytokines (secreted proteins and signal molecules) that are expressed and secreted by white blood cells (leukocytes) as well as some other body cells. Interleukin 10 (IL-10) is a protein that inhibits the synthesis of a number of cytokines, including IFN-gamma, IL-2, IL-3, TNF, and GM-CSF produced by activated macrophages and by helper T-cells. The terms “IL-10”, “IL10” and “Interleukin 10” are used interchangeably throughout the description. In arthritis, activated CD4 T helper cells and macrophages are believed to be the primary driving force behind joint inflammation. IL-10 can inhibit the synthesis of cytokines produced by said CD4 T-helper cells and macrophages.

IL1RN is a member of the interleukin 1 cytokine family. IL1RN is secreted by various types of cells including immune cells, epithelial cells, and adipocytes, and is a natural inhibitor of the pro-inflammatory effect of IL1β.

WNT3A is highly related to the WNT3 gene in sequence and protein function. WNT3A and WNT3 signal similarly through primarily the beta-catenin/Tcf pathway. WNT proteins are critical in tissue homeostasis, embryonic development, and disease. IGF1 is mainly secreted by the liver as a result of stimulation by growth hormone (GH). IGF1 is important for both the regulation of normal physiology, as well as a number of pathological states, including cancer. The IGF axis has been shown to play a role in the promotion of cell proliferation and the inhibition of cell death (apoptosis).

BMPs are known for their ability to induce the growth of bone and cartilage. The bone morphogenetic proteins (BMPs) are a family of secreted signaling molecules that can induce ectopic bone growth. BMP6 is able to induce all osteogenic markers in mesenchymal stem cells.

The MSCs transfected with mRNA encoding TGFβ3 can be injected into the subject before or after injection of MSCs transfected with a different mRNA construct. Furthermore, one MSC can be transfected with mRNA constructs encoding different proteins.

Thus, in some embodiments of the present disclosure, the cells are transfected with at least one mRNA construct, such as at least two different mRNA constructs, such as at least three different mRNA constructs, such as at least 4 different mRNA constructs, such as at least 5 different mRNA constructs, such as 6 different mRNA constructs.

In some embodiments of the present disclosure, the cells are transfected with an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 25 and one or more mRNA construct(s), such as one or more mRNA construct(s) comprising mRNA having at least 70%, such as at least 80%, such as at least 85%, such as at least 90%, such as at least 95%, such as at least 97%, such as at least 99%, such as 100% sequence identity to
a. SEQ ID NO: 31; or
b. SEQ ID NO: 1; or
c. SEQ ID NO: 19; or
d. SEQ ID NO: 13; or
e. SEQ ID NO: 7.

In some embodiments of the present disclosure, the cells are transfected with an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 25 and one or more mRNA construct(s) comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to
a. SEQ ID NO: 31; or
b. SEQ ID NO: 1; or
c. SEQ ID NO: 19; or
d. SEQ ID NO: 13; or
e. SEQ ID NO: 7.

In some embodiments of the present disclosure, the cells are transfected with an mRNA construct comprising mRNA encoding TGFβ3 of SEQ ID NO: 29 or a functional homologue of said TGFβ3 sharing at least 70%, such as at least 80%, for example at least 90%, such as at least 95% sequence identity therewith and one or more mRNA construct(s), such as one or more mRNA construct(s) comprising an mRNA construct encoding a factor of the group comprising:
a. WNT3a of SEQ ID NO: 35 or a functional homologue of said WNT3a sharing at least 70%, such as at least 80%, for example at least 90%, such as at least 95% sequence identity therewith; or
b. IL-10 of SEQ ID NO: 5 or a functional homologue of said IL-10 sharing at least 70%, such as at least 80%, for example at least 90%, such as at least 95% sequence identity therewith; or
c. IGF1 of SEQ ID NO: 23 or a functional homologue of said IGF1 sharing at least 70%, such as at least 80%, for example at least 90%, such as at least 95% sequence identity therewith;
d. BMP6 of SEQ ID NO: 17 or a functional homologue of said BMP6 sharing at least 70%, such as at least 80%, for example at least 90%, such as at least 95% sequence identity therewith;
e. IL1RN of SEQ ID NO: 11 or a functional homologue of said IL1RN sharing at least 70%, such as at least 80%, for example at least 90%, such as at least 95% sequence identity therewith.

In some embodiments of the present disclosure, the cells are transfected with an mRNA construct comprising mRNA encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith and one or more mRNA construct(s) comprising an mRNA construct encoding a factor of the group comprising:
a. WNT3a of SEQ ID NO: 35 or a functional homologue of said WNT3a sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
b. IL-10 of SEQ ID NO: 5 or a functional homologue of said IL-10 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
c. IGF1 of SEQ ID NO: 23 or a functional homologue of said IGF1 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith;
d. BMP6 of SEQ ID NO: 17 or a functional homologue of said BMP6 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith;
e. IL1RN of SEQ ID NO: 11 or a functional homologue of said IL1RN sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments of the present disclosure, the ratio between the mRNA construct encoding TGFβ3 and one of the mRNA constructs encoding one of the factors a to e is at least 1:0.5, such as at least 1:0.7, such as at least 1:0.9, such as at least 1:1, such as at least 1:1.1, such as at least 1:1.2, such as at least 1:1.5, such as at least 1:2.

In some embodiments of the present disclosure, the mRNA construct(s) comprise a sequence encoding a heterologous or native signal peptide.

In some embodiments of the present disclosure, WNT3a is encoded by an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 31 or SEQ ID NO: 32. In some embodiments of the present disclosure, WNT3a is encoded by an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 31 or SEQ ID NO: 32.

In some embodiments of the present disclosure, IL-10 is encoded by an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 1 or SEQ ID NO: 2. In some embodiments of the present disclosure, IL-10 is encoded by an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 1 or SEQ ID NO: 2.

In some embodiments of the present disclosure, IGF1 is encoded by an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 19 or SEQ ID NO: 20. In some embodiments of the present disclosure, IGF1 is encoded by an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 19 or SEQ ID NO: 20.

In some embodiments of the present disclosure, BMP6 is encoded by an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 13 or SEQ ID NO: 14. In some embodiments of the present disclosure, BMP6 is encoded by an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 13 or SEQ ID NO: 14.

In some embodiments of the present disclosure, IL1RN is encoded by an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 7 or SEQ ID NO: 8. In some embodiments of the present disclosure, IL1RN is encoded by an mRNA construct comprising mRNA having at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity to SEQ ID NO: 7 or SEQ ID NO: 8.

In some embodiments of the present disclosure, prior to administering the MSCs comprising an mRNA construct comprising mRNA encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding WNT3a of SEQ ID NO: 35 or a functional homologue of said WNT3a sharing at least 70%, such as at least 80%, for example at least 90%, such as at least 95% sequence identity therewith. In some embodiments of the present disclosure, prior to administering the MSCs comprising an mRNA construct comprising mRNA encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith, MSCs are administered comprising an mRNA construct encoding WNT3a of SEQ ID NO: 35 or a functional homologue of said WNT3a sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments of the present disclosure, prior to administering the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding IL-10 of SEQ ID NO: 5 or a functional homologue of said IL-10 sharing at least 70%, such as at least 80%, for example at least 90%, such as at least 95% sequence identity therewith. In some embodiments of the present disclosure, prior to administering the MSCs comprising an mRNA construct encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith, MSCs are administered comprising an mRNA construct encoding IL-10 of SEQ ID NO: 5 or a functional homologue of said IL-10 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments of the present disclosure, prior to administering the cells comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding IGF1 of SEQ ID NO: 23 or a functional homologue of said IGF1 sharing at least 70%, such as at least 80%, for example at least 90%, such as at least 95% sequence identity therewith. In some embodiments of the present disclosure, prior to administering the cells comprising an mRNA construct encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith, MSCs are administered comprising an mRNA construct encoding IGF1 of SEQ ID NO: 23 or a functional homologue of said IGF1 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments of the present disclosure, prior to administering the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding BMP6 of SEQ ID NO: 17 or a functional homologue of said BMP6 sharing at least 70%, such as at least 80%, for example at least 90%, such as at least 95% sequence identity therewith. In some embodiments of the present disclosure, prior to administering the MSCs comprising an mRNA construct encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith, MSCs are administered comprising an mRNA construct encoding BMP6 of SEQ ID NO: 17 or a functional homologue of said BMP6 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments of the present disclosure, prior to administering the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding IL1RN of SEQ ID NO: 11 or a functional homologue of said IL1RN sharing at least 70%, such as at least 80%, for example at least 90%, such as at least 95% sequence identity therewith. In some embodiments of the present disclosure, prior to administering the MSCs comprising an mRNA construct encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith, MSCs are administered comprising an mRNA construct encoding IL1RN of SEQ ID NO: 11 or a functional homologue of said IL1RN sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments of the present disclosure, the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith are administered at least 1 day, such as at least 2 days, such as at least 5 days, such as at least 1 week, such as at least 2 weeks, such as at least 3 weeks, such as at least 4 weeks after administration of MSCs comprising mRNA construct encoding
d. WNT3a of SEQ ID NO: 35 or a functional homologue of said WNT3a sharing at least 70%, such as at least 80%, for example at least 90%, such as at least 95% sequence identity therewith; or
e. IL-10 of SEQ ID NO: 5 or a functional homologue of said IL-10 sharing at least 70%, such as at least 80%, for example at least 90%, such as at least 95% sequence identity therewith; or
f. IGF1 of SEQ ID NO:23 or a functional homologue of said IGF1 sharing at least 70%, such as at least 80%, for example at least 90%, such as at least 95% sequence identity therewith;
g. BMP6 of SEQ ID NO: 17 or a functional homologue of said BMP6 sharing at least 70%, such as at least 80%, for example at least 90%, such as at least 95% sequence identity therewith; or
h. IL1RN of SEQ ID NO: 11 or a functional homologue of said IL1RN sharing at least 70%, such as at least 80%, for example at least 90%, such as at least 95% sequence identity therewith.

In some embodiments of the present disclosure, the MSCs comprising an mRNA construct encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith are administered at least 1 day, such as at least 2 days, such as at least 5 days, such as at least 1 week, such as at least 2 weeks, such as at least 3 weeks, such as at least 4 weeks after administration of MSCs comprising mRNA construct encoding
a. WNT3a of SEQ ID NO: 35 or a functional homologue of said WNT3a sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
b. IL-10 of SEQ ID NO: 5 or a functional homologue of said IL-10 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
c. IGF1 of SEQ ID NO:23 or a functional homologue of said IGF1 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith;
d. BMP6 of SEQ ID NO: 17 or a functional homologue of said BMP6 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
e. IL1RN of SEQ ID NO: 11 or a functional homologue of said IL1RN sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments of the present disclosure, the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith are administered at most 1 day, such as at most 2 days, such as at most 5 days, such as at most 1 week, such as at most 2 weeks, such as at most 3 weeks, such as at most 4 weeks after administration of MSCs comprising mRNA construct encoding
a. WNT3a of SEQ ID NO: 35 or a functional homologue of said WNT3a sharing at least 70%, such as at least 80%, for example at least 90%, such as at least 95% sequence identity therewith; or
b. IL-10 of SEQ ID NO: 5 or a functional homologue of said IL-10 sharing at least 70%, such as at least 80%, for example at least 90%, such as at least 95% sequence identity therewith; or
c. IGF1 of SEQ ID NO: 23 or a functional homologue of said IGF1 sharing at least 70%, such as at least 80%, for example at least 90%, such as at least 95% sequence identity therewith;
d. BMP6 of SEQ ID NO: 17 or a functional homologue of said BMP6 sharing at least 70%, such as at least 80%, for example at least 90%, such as at least 95% sequence identity therewith; or
e. IL1RN of SEQ ID NO: 11 or a functional homologue of said IL1RN sharing at least 70%, such as at least 80%, for example at least 90%, such as at least 95% sequence identity therewith.

In some embodiments of the present disclosure, the MSCs comprising an mRNA construct encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith are administered at most 1 day, such as at most 2 days, such as at most 5 days, such as at most 1 week, such as at most 2 weeks, such as at most 3 weeks, such as at most 4 weeks after administration of MSCs comprising mRNA construct encoding
a. WNT3a of SEQ ID NO: 35 or a functional homologue of said WNT3a sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
b. IL-10 of SEQ ID NO: 5 or a functional homologue of said IL-10 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
c. IGF1 of SEQ ID NO: 23 or a functional homologue of said IGF1 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith;
d. BMP6 of SEQ ID NO: 17 or a functional homologue of said BMP6 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
e. IL1RN of SEQ ID NO: 11 or a functional homologue of said IL1RN sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments of the present disclosure, after administering the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding WNT3a of SEQ ID NO: 35 or a functional homologue of said WNT3a sharing at least 70%, such as at least 80%, for example at least 90%, such as at least 95% sequence identity therewith. In some embodiments of the present disclosure, after administering the MSCs comprising an mRNA construct encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith, MSCs are administered comprising an mRNA construct encoding WNT3a of SEQ ID NO: 35 or a functional homologue of said WNT3a sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments of the present disclosure, after administering the cells comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding IL-10 of SEQ ID NO: 5 or a functional homologue of said IL-10 sharing at least 70%, such as at least 80%, for example at least 90%, such as at least 95% sequence identity therewith. In some embodiments of the present disclosure, after administering the cells comprising an mRNA construct encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith, MSCs are administered comprising an mRNA construct encoding IL-10 of SEQ ID NO: 5 or a functional homologue of said IL-10 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments of the present disclosure, after administering the cells comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding IGF1 of SEQ ID NO: 23 or a functional homologue of said IGF1 sharing at least 70%, such as at least 80%, for example at least 90%, such as at least 95% sequence identity therewith. In some embodiments of the present disclosure, after administering the cells comprising an mRNA construct encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith, MSCs are administered comprising an mRNA construct encoding IGF1 of SEQ ID NO: 23 or a functional homologue of said IGF1 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments of the present disclosure, after administering the cells comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding BMP6 of SEQ ID NO: 17 or a functional homologue of said BMP6 sharing at least 70%, such as at least 80%, for example at least 90%, such as at least 95% sequence identity therewith. In some embodiments of the present disclosure, after administering the cells comprising an mRNA construct encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith, MSCs are administered comprising an mRNA construct encoding BMP6 of SEQ ID NO: 17 or a functional homologue of said BMP6 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments of the present disclosure, after administering the cells comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding IL1RN of SEQ ID NO: 11 or a functional homologue of said IL1RN sharing at least 70%, such as at least 80%, for example at least 90%, such as at least 95% sequence identity therewith. In some embodiments of the present disclosure, after administering the cells comprising an mRNA construct encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith, MSCs are administered comprising an mRNA construct encoding IL1RN of SEQ ID NO: 11 or a functional homologue of said IL1RN sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments of the present disclosure, the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith are administered at least 1 day, such as at least 2 days, such as at least 5 days, such as at least 1 week, such as at least 2 weeks, such as at least 3 weeks, such as at least 4 weeks before administration of MSCs comprising mRNA construct encoding
a. WNT3a of SEQ ID NO: 35 or a functional homologue of said WNT3a sharing at least 70%, such as at least 80%, for example at least 90%, such as at least 95% sequence identity therewith; or
b. IL-10 of SEQ ID NO: 5 or a functional homologue of said IL-10 sharing at least 70%, such as at least 80%, for example at least 90%, such as at least 95% sequence identity therewith; or
c. IGF1 of SEQ ID NO: 23 or a functional homologue of said IGF1 sharing at least 70%, such as at least 80%, for example at least 90%, such as at least 95% sequence identity therewith;
d. BMP6 of SEQ ID NO: 17 a functional homologue of said BMP6 sharing at least 70%, such as at least 80%, for example at least 90%, such as at least 95% sequence identity therewith; or
e. IL1RN of SEQ ID NO: 11 or a functional homologue of said IL1RN sharing at least 70%, such as at least 80%, for example at least 90%, such as at least 95% sequence identity therewith.

In some embodiments of the present disclosure, the MSCs comprising an mRNA construct encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith are administered at least 1 day, such as at least 2 days, such as at least 5 days, such as at least 1 week, such as at least 2 weeks, such as at least 3 weeks, such as at least 4 weeks before administration of MSCs comprising mRNA construct encoding
a. WNT3a of SEQ ID NO: 35 or a functional homologue of said WNT3a sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
b. IL-10 of SEQ ID NO: 5 or a functional homologue of said IL-10 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
c. IGF1 of SEQ ID NO: 23 or a functional homologue of said IGF1 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith;
d. BMP6 of SEQ ID NO: 17 a functional homologue of said BMP6 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
e. IL1RN of SEQ ID NO: 11 or a functional homologue of said IL1RN sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments of the present disclosure, the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith are administered at most 1 day, such as at most 2 days, such as at most 5 days, such as at most 1 week, such as at most 2 weeks, such as at most 3 weeks, such as at most 4 weeks before administration of MSCs comprising mRNA construct encoding
a. WNT3a of SEQ ID NO: 35 or a functional homologue of said WNT3a sharing at least 70%, such as at least 80%, for example at least 90%, such as at least 95% sequence identity therewith; or
b. IL-10 of SEQ ID NO: 5 or a functional homologue of said IL-10 sharing at least 70%, such as at least 80%, for example at least 90%, such as at least 95% sequence identity therewith; or
c. IGF1 of SEQ ID NO: 23 or a functional homologue of said IGF1 sharing at least 70%, such as at least 80%, for example at least 90%, such as at least 95% sequence identity therewith;
d. BMP6 of SEQ ID NO: 17 or a functional homologue of said BMP6 sharing at least 70%, such as at least 80%, for example at least 90%, such as at least 95% sequence identity therewith; or
e. IL1RN of SEQ ID NO: 11 or a functional homologue of said IL1RN sharing at least 70%, such as at least 80%, for example at least 90%, such as at least 95% sequence identity therewith.

In some embodiments of the present disclosure, the MSCs comprising an mRNA construct encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith are administered at most 1 day, such as at most 2 days, such as at most 5 days, such as at most 1 week, such as at most 2 weeks, such as at most 3 weeks, such as at most 4 weeks before administration of MSCs comprising mRNA construct encoding
a. WNT3a of SEQ ID NO: 35 or a functional homologue of said WNT3a sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
b. IL-10 of SEQ ID NO: 5 or a functional homologue of said IL-10 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
c. IGF1 of SEQ ID NO: 23 or a functional homologue of said IGF1 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith;
d. BMP6 of SEQ ID NO: 17 or a functional homologue of said BMP6 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
e. IL1RN of SEQ ID NO: 11 or a functional homologue of said IL1RN sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments of the present disclosure, the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith are administered simultaneously with MSCs comprising mRNA construct encoding
a. WNT3a of SEQ ID NO: 35 or a functional homologue of said WNT3a sharing at least 70%, such as at least 80%, for example at least 90%, such as at least 95% sequence identity therewith; or
b. IL-10 of SEQ ID NO: 5 or a functional homologue of said IL-10 sharing at least 70%, such as at least 80%, for example at least 90%, such as at least 95% sequence identity therewith; or
c. IGF1 of SEQ ID NO: 23 or a functional homologue of said IGF1 sharing at least 70%, such as at least 80%, for example at least 90%, such as at least 95% sequence identity therewith;
d. BMP6 of SEQ ID NO: 17 or a functional homologue of said BMP6 sharing at least 70%, such as at least 80%, for example at least 90%, such as at least 95% sequence identity therewith; or
e. IL1RN of SEQ ID NO: 11 or a functional homologue of said IL1RN sharing at least 70%, such as at least 80%, for example at least 90%, such as at least 95% sequence identity therewith.

In some embodiments of the present disclosure, the MSCs comprising an mRNA construct encoding TGFβ3 of SEQ ID NO: 29 or a functional homologue of said TGFβ3 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith are administered simultaneously with MSCs comprising mRNA construct encoding
a. WNT3a of SEQ ID NO: 35 or a functional homologue of said WNT3a sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
b. IL-10 of SEQ ID NO: 5 or a functional homologue of said IL-10 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
c. IGF1 of SEQ ID NO: 23 or a functional homologue of said IGF1 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith;
d. BMP6 of SEQ ID NO: 17 or a functional homologue of said BMP6 sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith; or
e. IL1RN of SEQ ID NO: 11 or a functional homologue of said IL1RN sharing at least 70%, such as at least 72%, such as at least 74%, such as at least 75%, such as at least 77%, such as at least 79%, such as at least 80%, such as at least 82%, such as at least 84%, such as at least 85%, such as at least 87%, such as at least 89%, such as at least 90%, such as at least 92%, such as at least 94%, such as at least 95%, such as at least 96%, such as at least 97%, such as at least 98%, such as at least 99%, such as 100% sequence identity therewith.

In some embodiments, of the present disclosure, one MSC comprises at least 1×104 copies of the mRNA construct, such as at least 5×10⁴ copies of the mRNA construct, such as at least 1×10⁵ copies of the mRNA construct, such as at least 2×10⁵ copies of the mRNA construct, such as at least 3×10⁵ copies of the mRNA construct at the time of administration.

### EXAMPLES

### Example 1: Stability of Codon Optimized TGFβ3 modRNA in iMSCs

### Aim:

To investigate the stability of codon optimized TGFβ3 modRNA in iMSCs.

### Material and Methods:

iMSCs according to the iMSC differentiation protocol as described in WO2023/148556 were prepared. Cells between passage 5 (P5) to P10 were used for the electroporation. Codon optimized TGFβ3 modified mRNA (modRNA) were synthesized in vitro and then electroporated into iMSCs at the ratio of 5 μg modRNA per million cells. iMSCs were electroporated with the mRNA by using Lonza Nucleofector and the P1 Primary Cell 4D-Nucleofector X Kit, basically as described in WO2023/199113. The electroporated cells recovered in iMSC medium.

Cells were then seeded in cell culture plates until the time of harvest. Cell pellets and culture media supernatant were collected at 3 hours post transfection (hpt), 6 hpt, 12 hpt, 18 hpt, 1 day post transfection (dpt), 2 dpt, 3 dpt, 4 dpt, 5 dpt, 6 dpt and 7 dpt. At each timepoint, cell pellets were used to extract total RNA and analyze the retention of transfected modRNA inside iMSCs by using real-time quantitative PCR with modRNA specific primers.

### Results:

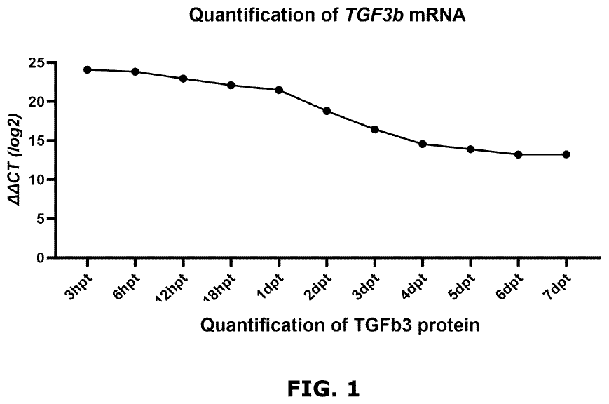

The modRNA level was stable on the first day after electroporation and decreased gradually in the following 6 days. At the end of the 7-day course, the iMSCs still maintained a certain level of modRNA (FIG. 1).

### Conclusion:

The inventors of the present disclosure have shown that codon optimized mRNA can be detected even 7 days post transfection in iMSCs.

### Example 2: Expression Levels of TGFβ3

### Aim:

To investigate the duration of TGFβ3 expression in iMSCs.

### Material and Methods:

The cells were prepared as described in example 1. The protein products of the transfected modRNA were quantified by ELISA on the culture media supernatant.

### Results:

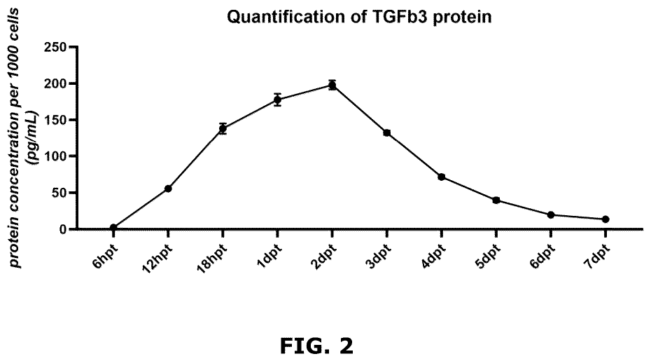

The protein products of the modRNA was first detected at 12hpt. The protein level increased to reach a plateau from 18 hours post transfection and declined gradually from 3dpt (days post transfection) (FIG. 2).

### Conclusion:

The inventors of the present disclosure have shown that a certain amount of TGFβ3 is expressed by the iMSCs even at 7dpt.

### Example 3: Effect of Cryopreservation of Transfected iMSCs on mRNA Stability

### Aim:

To investigate the effect of freezing and thawing of transfected iMSCs on the stability of TGFβ3 mRNA.

### Material and Methods:

The cells were prepared as described in example 1, except that the transfected cells were cryopreserved in liquid nitrogen after recovery in iMSC medium and weeks later thawed. The mRNA was measured as described in example 1.

### Results:

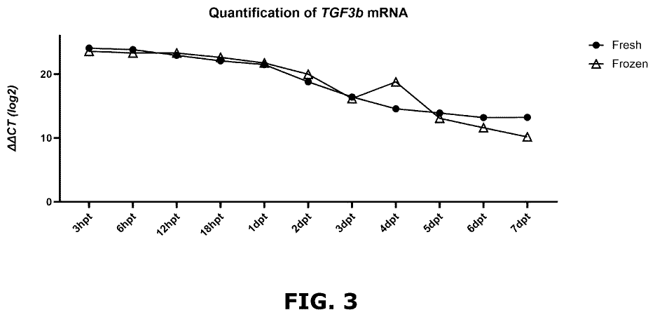

The mRNA level of the TGFβ3 mRNA was comparable across all timepoints after the freeze-and-thaw cycle compared to freshly prepared (never been frozen) iMSCs (FIG. 3).

### Conclusion:

The inventors of the present disclosure have shown that the transfected iMSCs can be frozen without diminishing the stability of the TGFβ3 mRNA.

### Example 4: Effect of Cryopreservation of Transfected iMSCs on Protein Expression

### Aim:

To investigate the effect of freeze and thaw on transfected iMSCs' TGFβ3 protein expression.

### Material and Methods:

The cells were prepared as described in example 1, except that the transfected cells were cryopreserved with freezing media in liquid nitrogen following a recovery period in iMSC medium and thawed weeks later. The protein expression was measured as described in example 2.

### Results:

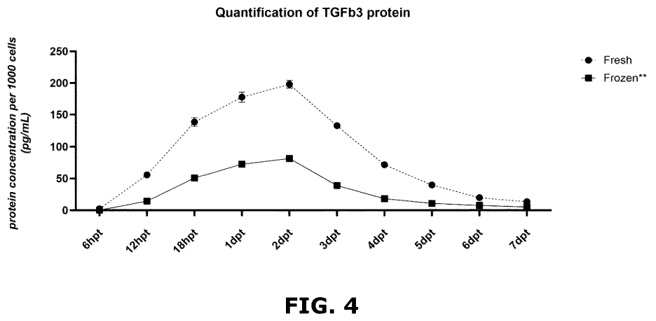

The TGFβ3 protein expression was comparatively lower across all timepoints after the freeze-and-thaw cycle when compared to never been frozen iMSCs (FIG. 4).

### Conclusion:

The inventors of the present disclosure have demonstrated that the transfected iMSCs can be frozen without diminishing the protein expression level of TGFβ3.

### Example 5: Upregulation of the Extracellular Matrix Protein Expression

### Aim:

To investigate the chondroprotective function of iMSCs electroporated with TGFβ3.

### Material and Methods:

Human articular chondrocytes (hACh) were seeded on glass coverslips in 12-well tissue culture plates to set up for the co-culture system with iMSCs. The hACh were seeded and maintained in Chondrocyte Growth Medium (Lonza). When reaching 80-90% confluence, cells were rinsed once with PBS and switched to 1 mL iMSC medium per well. Cell culture inserts (0.4 μm) were then placed into each well and covered with 0.5 mL of iMSC medium per insert. Control iMSCs (120k/inserts) and iMSCs electroporated with modRNA were seeded onto inserts with the density of 120k/insert. The co-culture system was maintained in 37° C. until the time of harvest; 3 days after co-culture initiation (Day 3) and on Day 7. At the end of hACh-iMSC co-culture on Day 3 and Day 7, hACh were fixed with 4% PFA followed by immunofluorescence staining with anti-ACAN antibody. Staining was imaged and analyzed by fluorescent microscope.

### Results:

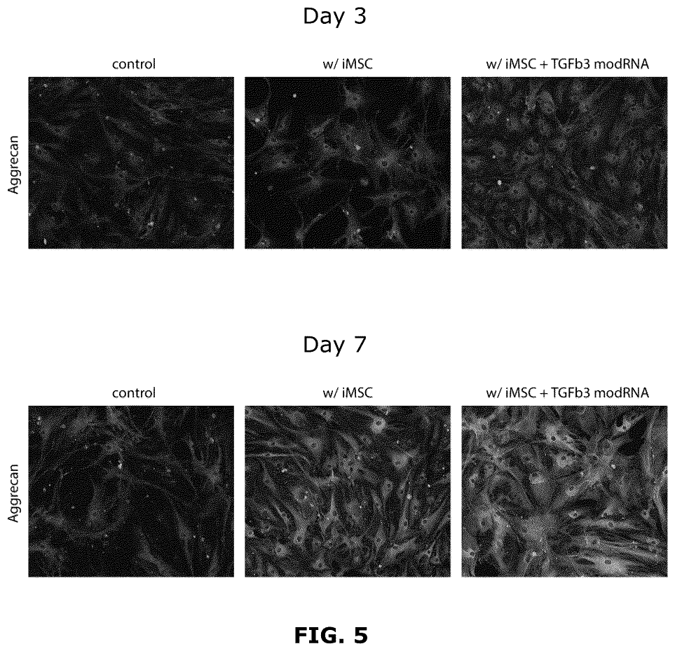

The intensity signals of anti-ACAN increased in hACh co-cultured with iMSCs electroporated with TGFβ3 modRNA, illustrating an upregulation of aggrecan expression in hACh. Moreover, such an increase is dose and time-dependent (FIG. 5).

### Conclusion:

The inventors of the present disclosure have shown a chondroprotective function of iMSCs electroporated with TGFβ3.

### Example 6: Promotion of the Chondrocyte Proliferation

### Aim:

To investigate, if iMSCs electroporated with TGFβ3 promote chondrocyte proliferation.

### Material and Methods:

Human articular chondrocytes (hACh) were seeded on glass coverslip in 12-well plates to set up the co-culture system with iMSCs as described in example 5. Following the co-culture of hACh with either iMSCs or iMSCs electroporated with TGFβ3 for 3 days, hACh were incubated with EdU containing medium for 4 hours in 37° C. The incorporation of EdU represents the genome synthesis during cell cycle, which indicates cells that are active in mitosis. After cultivation, cells were fixed in 4% PFA. The incorporation of EdU was detected by using Click-iT™ EdU Cell Proliferation Kit for Imaging (Thermo Fisher).

### Results:

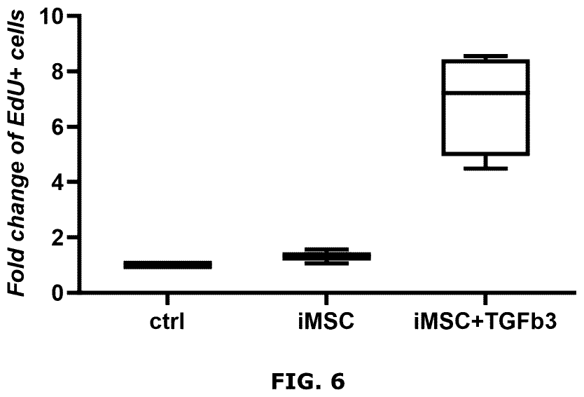

Compared to the control group (without the co-culture of any iMSCs), the number of EdU positive cells were significantly elevated in hACh co-cultured with iMSCs electroporated with TGFβ3 modRNA (FIG. 6).

### Conclusion:

The inventors of the present disclosure have shown that the proliferation of chondrocyte was enhanced by co-culturing the chondrocytes with transfected iMSCs. Thus, transfected iMSC can promote the chondrogenesis in hACh.

### Example 7: Functional Improvement of the Osteoarthritis Joints with TGFβ3 modRNA Electroporated iMSCs

### Aim:

To investigate the functional efficacy of the iMSCs electroporated with TGFβ3 in an in vivo OA disease model.

### Material and Methods:

The surgical operation of unilateral medial meniscal tear (MMT) and medial collateral ligament transection (MCLT) was performed in young adult male rats. An unoperated group was reserved as healthy group. Three weeks after surgery, intra-articular injection was performed with either 50 μl (1 million) or 50 μL (3 million) of iMSCs, iMSCs electroporated with modRNA as well as the cell suspension solution. Both iMSCs and iMSCs electroporated with modRNA were prepared and cryopreserved as described in examples 1 and 3. On the day of injection, cells were thawed and resuspended in cell suspension solution, which is PBS supplemented with 1% hyaluronic acid. The cell mixtures were used within two hours.

Animals were monitored closely after the surgery, and the static mechanical allodynia was determined by paw withdrawal threshold. Briefly, animals were placed in the modular holder cage until it can stand still with all four paws. Then a series of von Frey filaments with different weights were applied to the operated limb and a response to this filament was evaluated. A positive response included an abrupt withdrawal of the hind paw from the filament stimulus or flinching behaviour immediately following the removal of the stimulus. Each measurement started from a medium weight filament and consecutive filaments were applied by using an up-down method described by Chaplan and co-workers (Chaplan et al. 1994). After obtaining the first positive response, the paw withdrawal threshold was considered as the filament that preceded the next negative response.

### Results:

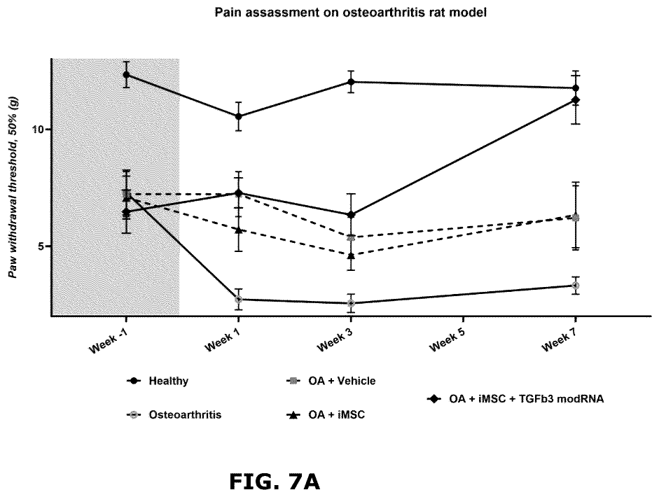

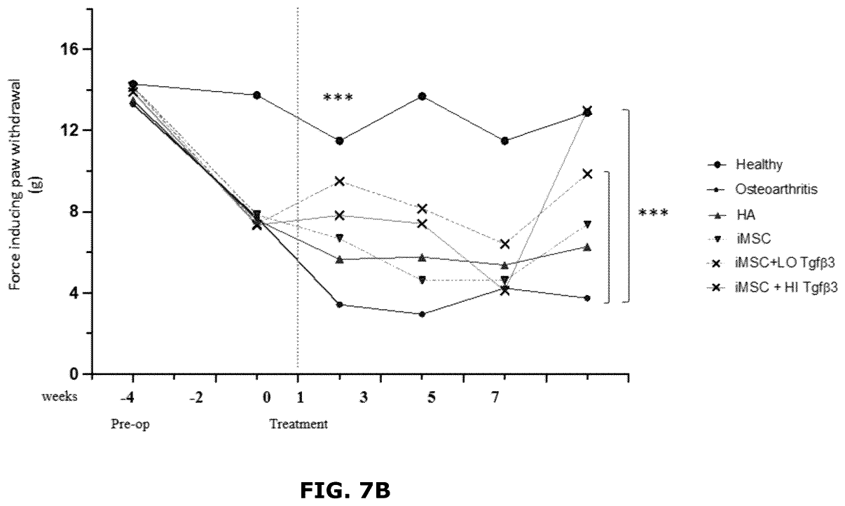

Healthy control animal could consistently bear a heavy weight of filament through the whole experiment (Healthy), while all surgical groups started experiencing significant pain on week −1 before injection (2 weeks after surgery). Untreated osteoarthritis disease group (Osteoarthritis group) experienced increased pain from week −1 to week 1, and the pain remained at that level until the end of the experiment. Surgical (OA disease) groups injected with cell suspension solution (OA+hyaluronic acid as vehicle) and iMSCs (OA+iMSC) on week 0 (border of grey box) had some mild pain relief from week 1, however such a pain relief was not as prominent as in the surgical group injected with iMSCs electroporated with TGFβ3. The pain relief and functional improvement was achieved by one single injection and the effect sustained for at least 7 weeks (FIG. 7A). The results indicate that both cell doses produce comparable effects on the reduction of joint pain (FIG. 7B).

### Conclusion:

The inventors of the present disclosure have shown that iMSCs electroporated with TGFβ3 modRNA can significantly reduce the joint pain in osteoarthritis rats and improve the joint function.

### Example 8: Cartilage Histology Stainings

### Aim:

To investigate the cartilage degeneration effect of the iMSCs electroporated with TGFβ3.

### Material and Methods:

At the end of in-life phase on 3 weeks after administration (Week 3, n=13 animals) and 7 weeks after administration (Week 7, n=9 animals), knee joints were harvested from each rat. Joint samples including distal femur and proximal tibia were then detached and immediately fixed in 4% PFA for two to three days, after which samples were transferred into 70% EtOH for further histological processing and analysis. Joint samples were first decalcified in 20% aqueous ethylenediaminetetraacetic acid (EDTA; VWR International, Leuven, Belgium), dehydrated in an increasing series of EtOH concentrations, defatted in xylene and processed for embedding in paraffin (Tissue-Tek Paraffin Wax; Sakura Finetek, Alphen aan den Rijn, The Netherlands). Frontal sections with a thickness of 4 μm were obtained from six levels per knee joint at approximately 200 μm distance between each level. Two microscope sections in each of the six levels were deparaffinized, rehydrated, stained with toluidine blue, dehydrated, embedded with xylene-based mountain medium and then covered with coverslips for histological OA assessment. Histological assessment of OA changes in the medial compartment and tibial plateau of the ipsilateral right knee joints was performed according to the guidelines recommended by the OARSI histopathology initiative (Gerwin et al. 2010).

### Results:

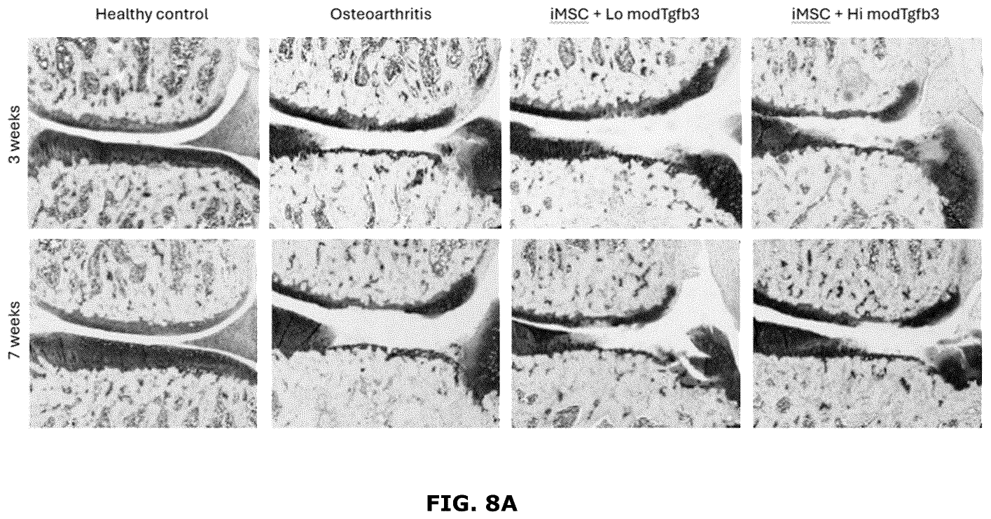

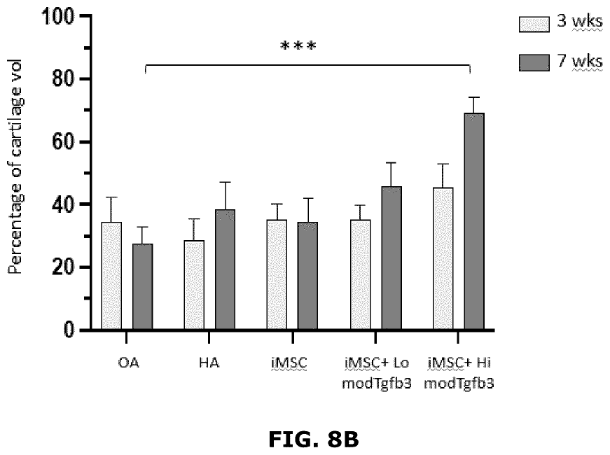

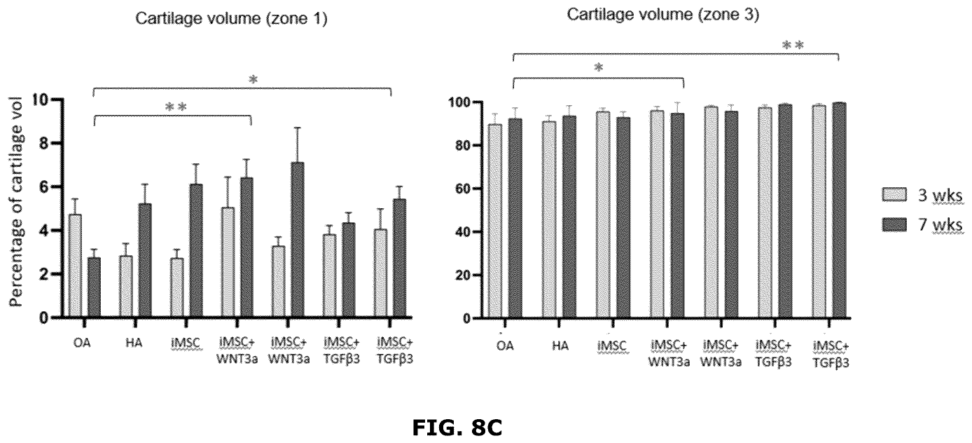

As shown in FIGS. 8A-8C of histological staining, comparing to healthy control the osteoarthritis group developed severe cartilage defect from 3 weeks on. When the OA model was treated with iMSC+TGFβ3 modRNA (both low and high dose), cartilage defect was significant improved after 3 weeks and improved even further on 7 weeks.

### Conclusion:

The inventors of the present disclosure have shown that the cartilage volume of joints from rats treated with iMSCs electroporated with TGFβ3 modRNA is 42% structural improved after 7 weeks compared to non-treated rats.

### Example 9: MRNA Levels Over Time

### Aim:

To investigate the duration of TGFβ3 mRNA inside transfected cells.

### Material and Methods:

The cells were prepared as described in example 1. The mRNA levels were measured via Quantitative Reverse Transcription PCR (qRT-PCR).

### Results:

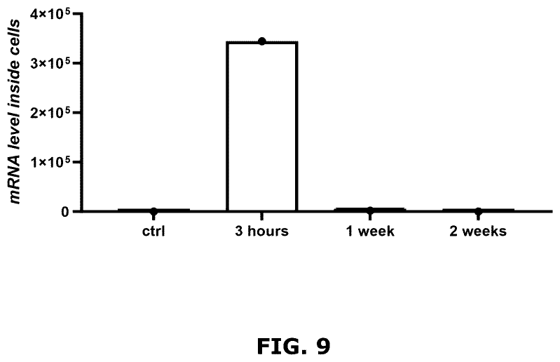

The results (FIG. 9) demonstrate that the cells comprised close to 3500× more TGFβ3 mRNA construct 3 hours after transfection compared to 14 days after transfection.

Sequence overview
SEQ ID NO: 1:
IL-10 optimized mRNA without signal peptide
AGCCCTGGCCAGGGCACCCAATCTGAAAATTCCTGCACCCACTTCCCCGGCAACCTGCCTAATAT
GCTGAGAGATCTGCGCGACGCATTCAGCAGAGTGAAGACATTCTTCCAAATGAAGGACCAGCTGG
ACAACCTGCTGCTTAAAGAGAGCCTGCTGGAAGATTTTAAGGGATATCTGGGATGTCAGGCTCTG
TCTGAGATGATCCAGTTCTACCTGGAAGAGGTAATGCCTCAGGCCGAGAACCAGGACCCCGATAT
TAAGGCCCATGTGAACTCCCTGGGCGAGAATCTGAAGACCCTGCGGCTGAGACTGCGGAGATGCC
ACAGATTCCTGCCATGTGAAAACAAAAGCAAGGCCGTGGAACAGGTCAAGAACGCATTTAACAAG
CTGCAGGAGAAGGGCATCTACAAGGCTATGAGCGAGTTCGACATCTTCATCAACTACATCGAGGC
CTACATGACAATGAAAATCAGAAACTGA
SEQ ID NO: 2:
IL-10 optimized mRNA with signal peptide
ATGCACAGCAGCGCCCTGCTGTGCTGCCTGGTGCTGCTCACCGGAGTGCGGGCCAGCCCTGGCCA
GGGCACCCAATCTGAAAATTCCTGCACCCACTTCCCCGGCAACCTGCCTAATATGCTGAGAGATC
TGCGCGACGCATTCAGCAGAGTGAAGACATTCTTCCAAATGAAGGACCAGCTGGACAACCTGCTG
CTTAAAGAGAGCCTGCTGGAAGATTTTAAGGGATATCTGGGATGTCAGGCTCTGTCTGAGATGAT
CCAGTTCTACCTGGAAGAGGTAATGCCTCAGGCCGAGAACCAGGACCCCGATATTAAGGCCCATG
TGAACTCCCTGGGCGAGAATCTGAAGACCCTGCGGCTGAGACTGCGGAGATGCCACAGATTCCTG
CCATGTGAAAACAAAAGCAAGGCCGTGGAACAGGTCAAGAACGCATTTAACAAGCTGCAGGAGAA
GGGCATCTACAAGGCTATGAGCGAGTTCGACATCTTCATCAACTACATCGAGGCCTACATGACAA
TGAAAATCAGAAACTGA
SEQ ID NO: 3:
IL-10 mRNA without signal peptide
AGCCCAGGCCAGGGCACCCAGTCTGAGAACAGCTGCACCCACTTCCCAGGCAACCTGCCTAACAT
GCTTCGAGATCTCCGAGATGCCTTCAGCAGAGTGAAGACTTTCTTTCAAATGAAGGATCAGCTGG
ACAACTTGTTGTTAAAGGAGTCCTTGCTGGAGGACTTTAAGGGTTACCTGGGTTGCCAAGCCTTG
TCTGAGATGATCCAGTTTTACCTGGAGGAGGTGATGCCCCAAGCTGAGAACCAAGACCCAGACAT
CAAGGCGCATGTGAACTCCCTGGGGGAGAACCTGAAGACCCTCAGGCTGAGGCTACGGCGCTGTC
ATCGATTTCTTCCCTGTGAAAACAAGAGCAAGGCCGTGGAGCAGGTGAAGAATGCCTTTAATAAG
CTCCAAGAGAAAGGCATCTACAAAGCCATGAGTGAGTTTGACATCTTCATCAACTACATAGAAGC
CTACATGACAATGAAGATACGAAACTGA
SEQ ID NO: 4:
IL-10 mRNA with signal peptide
ATGCACAGCTCAGCACTGCTCTGTTGCCTGGTCCTCCTGACTGGGGTGAGGGCCAGCCCAGGCCA
GGGCACCCAGTCTGAGAACAGCTGCACCCACTTCCCAGGCAACCTGCCTAACATGCTTCGAGATC
TCCGAGATGCCTTCAGCAGAGTGAAGACTTTCTTTCAAATGAAGGATCAGCTGGACAACTTGTTG
TTAAAGGAGTCCTTGCTGGAGGACTTTAAGGGTTACCTGGGTTGCCAAGCCTTGTCTGAGATGAT
CCAGTTTTACCTGGAGGAGGTGATGCCCCAAGCTGAGAACCAAGACCCAGACATCAAGGCGCATG
TGAACTCCCTGGGGGAGAACCTGAAGACCCTCAGGCTGAGGCTACGGCGCTGTCATCGATTTCTT
CCCTGTGAAAACAAGAGCAAGGCCGTGGAGCAGGTGAAGAATGCCTTTAATAAGCTCCAAGAGAA
AGGCATCTACAAAGCCATGAGTGAGTTTGACATCTTCATCAACTACATAGAAGCCTACATGACAA
TGAAGATACGAAACTGA
SEQ ID NO: 5:
amino acid sequence of IL-10 without signal peptide
SPGQGTQSENSCTHFPGNLPNMLRDLRDAFSRVKTFFQMKDQLDNLLLKESLLEDFKGYLGCQAL
SEMIQFYLEEVMPQAENQDPDIKAHVNSLGENLKTLRLRLRRCHRFLPCENKSKAVEQVKNAFNK
LQEKGIYKAMSEFDIFINYIEAYMTMKIRN*
SEQ ID NO: 6:
amino acid sequence of IL-10 with signal peptide
MHSSALLCCLVLLTGVRASPGQGTQSENSCTHFPGNLPNMLRDLRDAFSRVKTFFQMKDQLDNLL
LKESLLEDFKGYLGCQALSEMIQFYLEEVMPQAENQDPDIKAHVNSLGENLKTLRLRLRRCHRFL
PCENKSKAVEQVKNAFNKLQEKGIYKAMSEFDIFINYIEAYMTMKIRN*
SEQ ID NO: 7:
IL1RN optimized mRNA without signal peptide
AGACCTAGCGGAAGAAAGAGCAGCAAGATGCAGGCCTTTAGAATCTGGGATGTTAATCAGAAAAC
CTTCTACCTGCGGAACAACCAGCTGGTCGCCGGCTACCTGCAGGGCCCCAACGTGAATCTGGAAG
AGAAGATCGACGTGGTGCCAATCGAGCCCCACGCCCTGTTCCTGGGCATCCATGGCGGAAAAATG
TGCCTGAGCTGCGTGAAGTCCGGCGACGAAACCAGACTGCAACTCGAGGCCGTGAACATTACCGA
CCTGTCTGAGAACCGGAAGCAAGACAAGCGCTTCGCATTCATCAGAAGCGACAGCGGCCCTACCA
CAAGCTTCGAGTCTGCCGCTTGTCCTGGCTGGTTCCTGTGCACAGCCATGGAAGCTGATCAGCCT
GTGTCCCTGACCAACATGCCTGATGAAGGCGTGATGGTGACCAAGTTCTACTTCCAGGAGGACGA
GTGA
SEQ ID NO: 8:
IL1RN optimized mRNA with signal peptide
ATGGAAATCTGCAGGGGCCTGAGATCTCACCTGATCACCCTGCTGCTTTTTCTGTTCCACAGCGA
GACAATCTGTAGACCTAGCGGAAGAAAGAGCAGCAAGATGCAGGCCTTTAGAATCTGGGATGTTA
ATCAGAAAACCTTCTACCTGCGGAACAACCAGCTGGTCGCCGGCTACCTGCAGGGCCCCAACGTG
AATCTGGAAGAGAAGATCGACGTGGTGCCAATCGAGCCCCACGCCCTGTTCCTGGGCATCCATGG
CGGAAAAATGTGCCTGAGCTGCGTGAAGTCCGGCGACGAAACCAGACTGCAACTCGAGGCCGTGA
ACATTACCGACCTGTCTGAGAACCGGAAGCAAGACAAGCGCTTCGCATTCATCAGAAGCGACAGC
GGCCCTACCACAAGCTTCGAGTCTGCCGCTTGTCCTGGCTGGTTCCTGTGCACAGCCATGGAAGC
TGATCAGCCTGTGTCCCTGACCAACATGCCTGATGAAGGCGTGATGGTGACCAAGTTCTACTTCC
AGGAGGACGAGTGA
SEQ ID NO: 9:
IL1RN mRNA without signal peptide
CGACCCTCTGGGAGAAAATCCAGCAAGATGCAAGCCTTCAGAATCTGGGATGTTAACCAGAAGAC
CTTCTATCTGAGGAACAACCAACTAGTTGCTGGATACTTGCAAGGACCAAATGTCAATTTAGAAG
AAAAGATAGATGTGGTACCCATTGAGCCTCATGCTCTGTTCTTGGGAATCCATGGAGGGAAGATG
TGCCTGTCCTGTGTCAAGTCTGGTGATGAGACCAGACTCCAGCTGGAGGCAGTTAACATCACTGA
CCTGAGCGAGAACAGAAAGCAGGACAAGCGCTTCGCCTTCATCCGCTCAGACAGTGGCCCCACCA
CCAGTTTTGAGTCTGCCGCCTGCCCCGGTTGGTTCCTCTGCACAGCGATGGAAGCTGACCAGCCC
GTCAGCCTCACCAATATGCCTGACGAAGGCGTCATGGTCACCAAATTCTACTTCCAGGAGGACGA
GTAG
SEQ ID NO: 10:
IL1RN mRNA with signal peptide
ATGGAAATCTGCAGAGGCCTCCGCAGTCACCTAATCACTCTCCTCCTCTTCCTGTTCCATTCAGA
GACGATCTGCCGACCCTCTGGGAGAAAATCCAGCAAGATGCAAGCCTTCAGAATCTGGGATGTTA
ACCAGAAGACCTTCTATCTGAGGAACAACCAACTAGTTGCTGGATACTTGCAAGGACCAAATGTC
AATTTAGAAGAAAAGATAGATGTGGTACCCATTGAGCCTCATGCTCTGTTCTTGGGAATCCATGG
AGGGAAGATGTGCCTGTCCTGTGTCAAGTCTGGTGATGAGACCAGACTCCAGCTGGAGGCAGTTA
ACATCACTGACCTGAGCGAGAACAGAAAGCAGGACAAGCGCTTCGCCTTCATCCGCTCAGACAGT
GGCCCCACCACCAGTTTTGAGTCTGCCGCCTGCCCCGGTTGGTTCCTCTGCACAGCGATGGAAGC
TGACCAGCCCGTCAGCCTCACCAATATGCCTGACGAAGGCGTCATGGTCACCAAATTCTACTTCC
AGGAGGACGAGTAG
SEQ ID NO: 11:
amino acid sequence of IL1RN without signal peptide
RPSGRKSSKMQAFRIWDVNQKTFYLRNNQLVAGYLQGPNVNLEEKIDVVPIEPHALFLGIHGGKM
CLSCVKSGDETRLQLEAVNITDLSENRKQDKRFAFIRSDSGPTTSFESAACPGWFLCTAMEADQP
VSLTNMPDEGVMVTKFYFQEDE*
SEQ ID NO: 12:
amino acid sequence of IL1RN with signal peptide
MEICRGLRSHLITLLLFLFHSETICRPSGRKSSKMQAFRIWDVNQKTFYLRNNQLVAGYLQGPNV
NLEEKIDVVPIEPHALFLGIHGGKMCLSCVKSGDETRLQLEAVNITDLSENRKQDKRFAFIRSDS
GPTTSFESAACPGWFLCTAMEADQPVSLTNMPDEGVMVTKFYFQEDE*
SEQ ID NO: 13:
BMP6 optimized mRNA without signal peptide
TGTTGCGGACCACCTCCACTTAGACCACCATTGCCAGCAGCTGCAGCTGCCGCAGCCGGTGGACA
GCTGCTGGGAGATGGCGGCTCTCCTGGCAGGACCGAGCAGCCTCCTCCAAGCCCTCAATCTTCGA
GCGGCTTCCTGTACCGGAGACTCAAAACCCAGGAGAAGCGGGAAATGCAGAAAGAGATCCTGAGC
GTGCTGGGCCTGCCTCACCGGCCCAGACCTCTGCACGGCCTGCAGCAACCGCAGCCACCAGCCCT
GCGGCAACAGGAAGAGCAGCAACAGCAACAGCAACTGCCTAGAGGCGAGCCTCCTCCTGGCAGAC
TGAAATCTGCTCCTCTGTTCATGCTGGATCTGTATAACGCCCTGAGCGCCGATAACGACGAGGAC
GGCGCCTCTGAGGGAGAAAGACAACAGAGCTGGCCTCATGAGGCAGCCAGCAGCTCCCAGAGAAG
ACAGCCTCCTCCAGGAGCTGCACACCCTCTGAATAGAAAGAGCCTGCTGGCTCCTGGTTCCGGCA
GCGGCGGAGCCTCCCCCCTGACCAGCGCTCAGGACAGCGCTTTTCTGAACGACGCCGACATGGTT
ATGAGCTTCGTGAACCTGGTGGAATACGATAAGGAATTCAGCCCTAGACAGCGGCACCACAAGGA
GTTCAAGTTCAACCTGTCTCAGATCCCCGAGGGCGAGGTGGTGACAGCCGCCGAGTTCAGAATCT
ATAAGGACTGCGTGATGGGCAGCTTCAAAAACCAGACCTTCCTGATCTCCATCTACCAGGTGCTG
CAAGAACACCAGCATCGGGATAGCGACCTGTTCCTGCTGGACACCAGAGTGGTGTGGGCCAGCGA
AGAAGGCTGGCTGGAATTTGACATCACCGCCACAAGCAACCTGTGGGTCGTCACACCTCAGCACA
ACATGGGACTGCAGCTGAGCGTGGTTACCCGCGACGGCGTGCACGTGCACCCCAGAGCCGCAGGC
CTGGTGGGCCGGGACGGACCTTACGACAAGCAGCCTTTCATGGTGGCCTTCTTCAAGGTGTCCGA
GGTGCACGTGCGGACCACCCGGTCTGCCTCATCTAGACGGAGACAGCAGAGCAGAAACAGAAGCA
CACAGTCTCAGGATGTGGCCAGGGTGAGCAGCGCCTCTGACTACAACAGCAGCGAACTGAAGACA
GCATGCCGGAAGCACGAGCTGTACGTGTCCTTCCAGGATCTGGGCTGGCAGGACTGGATCATCGC
CCCCAAGGGCTACGCCGCTAACTACTGCGACGGAGAGTGCAGCTTTCCTCTCAACGCCCACATGA
ACGCCACCAACCACGCCATTGTGCAGACCCTGGTGCACCTGATGAATCCCGAGTACGTGCCAAAG
CCCTGTTGCGCCCCTACAAAGCTGAATGCCATCAGCGTGCTGTACTTCGACGATAATTCTAATGT
GATCCTGAAGAAGTACAGGAACATGGTGGTCAGAGCTTGTGGTTGTCACTGA
SEQ ID NO: 14:
BMP6 optimized mRNA with signal peptide
ATGCCTGGCCTGGGCAGAAGAGCCCAGTGGCTGTGCTGGTGGTGGGGACTGCTCTGTAGCTGTTG
CGGACCACCTCCACTTAGACCACCATTGCCAGCAGCTGCAGCTGCCGCAGCCGGTGGACAGCTGC
TGGGAGATGGGGGCTCTCCTGGCAGGACCGAGCAGCCTCCTCCAAGCCCTCAATCTTCGAGCGGC
TTCCTGTACCGGAGACTCAAAACCCAGGAGAAGCGGGAAATGCAGAAAGAGATCCTGAGCGTGCT
GGGCCTGCCTCACCGGCCCAGACCTCTGCACGGCCTGCAGCAACCGCAGCCACCAGCCCTGCGGC
AACAGGAAGAGCAGCAACAGCAACAGCAACTGCCTAGAGGCGAGCCTCCTCCTGGCAGACTGAAA
TCTGCTCCTCTGTTCATGCTGGATCTGTATAACGCCCTGAGCGCCGATAACGACGAGGACGGCGC
CTCTGAGGGAGAAAGACAACAGAGCTGGCCTCATGAGGCAGCCAGCAGCTCCCAGAGAAGACAGC
CTCCTCCAGGAGCTGCACACCCTCTGAATAGAAAGAGCCTGCTGGCTCCTGGTTCCGGCAGCGGC
GGAGCCTCCCCCCTGACCAGCGCTCAGGACAGCGCTTTTCTGAACGACGCCGACATGGTTATGAG
CTTCGTGAACCTGGTGGAATACGATAAGGAATTCAGCCCTAGACAGCGGCACCACAAGGAGTTCA
AGTTCAACCTGTCTCAGATCCCCGAGGGCGAGGTGGTGACAGCCGCCGAGTTCAGAATCTATAAG
GACTGCGTGATGGGCAGCTTCAAAAACCAGACCTTCCTGATCTCCATCTACCAGGTGCTGCAAGA
ACACCAGCATCGGGATAGCGACCTGTTCCTGCTGGACACCAGAGTGGTGTGGGCCAGCGAAGAAG
GCTGGCTGGAATTTGACATCACCGCCACAAGCAACCTGTGGGTCGTCACACCTCAGCACAACATG
GGACTGCAGCTGAGCGTGGTTACCCGCGACGGCGTGCACGTGCACCCCAGAGCCGCAGGCCTGGT
GGGCCGGGACGGACCTTACGACAAGCAGCCTTTCATGGTGGCCTTCTTCAAGGTGTCCGAGGTGC
ACGTGCGGACCACCCGGTCTGCCTCATCTAGACGGAGACAGCAGAGCAGAAACAGAAGCACACAG
TCTCAGGATGTGGCCAGGGTGAGCAGCGCCTCTGACTACAACAGCAGCGAACTGAAGACAGCATG
CCGGAAGCACGAGCTGTACGTGTCCTTCCAGGATCTGGGCTGGCAGGACTGGATCATCGCCCCCA
AGGGCTACGCCGCTAACTACTGCGACGGAGAGTGCAGCTTTCCTCTCAACGCCCACATGAACGCC
ACCAACCACGCCATTGTGCAGACCCTGGTGCACCTGATGAATCCCGAGTACGTGCCAAAGCCCTG
TTGCGCCCCTACAAAGCTGAATGCCATCAGCGTGCTGTACTTCGACGATAATTCTAATGTGATCC
TGAAGAAGTACAGGAACATGGTGGTCAGAGCTTGTGGTTGTCACTGA
SEQ ID NO: 15:
BMP6 mRNA without signal peptide
TGCTGCGGGCCCCCGCCGCTGCGGCCGCCCTTGCCCGCTGCCGCGGCCGCCGCCGCCGGGGGGCA
GCTGCTGGGGGACGGCGGGAGCCCCGGCCGCACGGAGCAGCCGCCGCCGTCGCCGCAGTCCTCCT
CGGGCTTCCTGTACCGGGGCTCAAGACGCAGGAGAAGCGGGAGATGCAGAAGGAGATCTTGTCGG
TGCTGGGGCTCCCGCACCGGCCCCGGCCCCTGCACGGCCTCCAACAGCCGCAGCCCCCGGCGCTC
CGGCAGCAGGAGGAGCAGCAGCAGCAGCAGCAGCTGCCTCGCGGAGAGCCCCCTCCCGGGCGACT
GAAGTCCGCGCCCCTCTTCATGCTGGATCTGTACAACGCCCTGTCCGCCGACAACGACGAGGACG
GGGCGTCGGAGGGGGAGAGGCAGCAGTCCTGGCCCCACGAAGCAGCCAGCTCGTCCCAGCGTCGG
CAGCCGCCCCCGGGCGCCGCGCACCCGCTCAACCGCAAGAGCCTTCTGGCCCCCGGATCTGGCAG
CGGCGGCGCGTCCCCACTGACCAGCGCGCAGGACAGCGCCTTCCTCAACGACGCGGACATGGTCA
TGAGCTTTGTGAACCTGGTGGAGTACGACAAGGAGTTCTCCCCTCGTCAGCGACACCACAAAGAG
TTCAAGTTCAACTTATCCCAGATTCCTGAGGGTGAGGTGGTGACGGCTGCAGAATTCCGCATCTA
CAAGGACTGTGTTATGGGGAGTTTTAAAAACCAAACTTTTCTTATCAGCATTTATCAAGTCTTAC
AGGAGCATCAGCACAGAGACTCTGACCTGTTTTTGTTGGACACCCGTGTAGTATGGGCCTCAGAA
GAAGGCTGGCTGGAATTTGACATCACGGCCACTAGCAATCTGTGGGTTGTGACTCCACAGCATAA
CATGGGGCTTCAGCTGAGCGTGGTGACAAGGGATGGAGTCCACGTCCACCCCCGAGCCGCAGGCC
TGGTGGGCAGAGACGGCCCTTACGACAAGCAGCCCTTCATGGTGGCTTTCTTCAAAGTGAGTGAG
GTGCACGTGCGCACCACCAGGTCAGCCTCCAGCCGGCGCCGACAACAGAGTCGTAATCGCTCTAC
CCAGTCCCAGGACGTGGCGCGGGTCTCCAGTGCTTCAGATTACAACAGCAGTGAATTGAAAACAG
CCTGCAGGAAGCATGAGCTGTATGTGAGTTTCCAAGACCTGGGATGGCAGGACTGGATCATTGCA
CCCAAGGGCTATGCTGCCAATTACTGTGATGGAGAATGCTCCTTCCCACTCAACGCACACATGAA
TGCAACCAACCACGCGATTGTGCAGACCTTGGTTCACCTTATGAACCCCGAGTATGTCCCCAAAC
CGTGCTGTGCGCCAACTAAGCTAAATGCCATCTCGGTTCTTTACTTTGATGACAACTCCAATGTC
ATTCTGAAAAAATACAGGAATATGGTTGTAAGAGCTTGTGGATGCCACTAA
SEQ ID NO: 16:
BMP6 mRNA with signal peptide
ATGCCGGGGCTGGGGCGGAGGGCGCAGTGGCTGTGCTGGTGGTGGGGGCTGCTGTGCAGCTGCTG
CGGGCCCCCGCCGCTGCGGCCGCCCTTGCCCGCTGCCGCGGCCGCCGCCGCCGGGGGGCAGCTGC
TGGGGGACGGCGGGAGCCCCGGCCGCACGGAGCAGCCGCCGCCGTCGCCGCAGTCCTCCTCGGGC
TTCCTGTACCGGCGGCTCAAGACGCAGGAGAAGCGGGAGATGCAGAAGGAGATCTTGTCGGTGCT
GGGGCTCCCGCACCGGCCCCGGCCCCTGCACGGCCTCCAACAGCCGCAGCCCCCGGCGCTCCGGC
AGCAGGAGGAGCAGCAGCAGCAGCAGCAGCTGCCTCGCGGAGAGCCCCCTCCCGGGCGACTGAAG
TCCGCGCCCCTCTTCATGCTGGATCTGTACAACGCCCTGTCCGCCGACAACGACGAGGACGGGGC
GTCGGAGGGGGAGAGGCAGCAGTCCTGGCCCCACGAAGCAGCCAGCTCGTCCCAGCGTCGGCAGC
CGCCCCCGGGCGCCGCGCACCCGCTCAACCGCAAGAGCCTTCTGGCCCCCGGATCTGGCAGCGGC
GGCGCGTCCCCACTGACCAGCGCGCAGGACAGCGCCTTCCTCAACGACGCGGACATGGTCATGAG
CTTTGTGAACCTGGTGGAGTACGACAAGGAGTTCTCCCCTCGTCAGCGACACCACAAAGAGTTCA
AGTTCAACTTATCCCAGATTCCTGAGGGTGAGGTGGTGACGGCTGCAGAATTCCGCATCTACAAG
GACTGTGTTATGGGGAGTTTTAAAAACCAAACTTTTCTTATCAGCATTTATCAAGTCTTACAGGA
GCATCAGCACAGAGACTCTGACCTGTTTTTGTTGGACACCCGTGTAGTATGGGCCTCAGAAGAAG
GCTGGCTGGAATTTGACATCACGGCCACTAGCAATCTGTGGGTTGTGACTCCACAGCATAACATG
GGGCTTCAGCTGAGCGTGGTGACAAGGGATGGAGTCCACGTCCACCCCCGAGCCGCAGGCCTGGT
GGGCAGAGACGGCCCTTACGACAAGCAGCCCTTCATGGTGGCTTTCTTCAAAGTGAGTGAGGTGC
ACGTGCGCACCACCAGGTCAGCCTCCAGCCGGCGCCGACAACAGAGTCGTAATCGCTCTACCCAG
TCCCAGGACGTGGCGCGGGTCTCCAGTGCTTCAGATTACAACAGCAGTGAATTGAAAACAGCCTG
CAGGAAGCATGAGCTGTATGTGAGTTTCCAAGACCTGGGATGGCAGGACTGGATCATTGCACCCA
AGGGCTATGCTGCCAATTACTGTGATGGAGAATGCTCCTTCCCACTCAACGCACACATGAATGCA
ACCAACCACGCGATTGTGCAGACCTTGGTTCACCTTATGAACCCCGAGTATGTCCCCAAACCGTG
CTGTGCGCCAACTAAGCTAAATGCCATCTCGGTTCTTTACTTTGATGACAACTCCAATGTCATTC
TGAAAAAATACAGGAATATGGTTGTAAGAGCTTGTGGATGCCACTAA
SEQ ID NO: 17:
amino acid sequence of BMP6 without signal peptide
MPGLGRRAQWLCWWWGLLCSCCGPPPLRPPLPAAAAAAAGGQLLGDGGSPGRTEQPPPSPQSSSG
FLYRRLKTQEKREMQKEILSVLGLPHRPRPLHGLQQPQPPALRQQEEQQQQQQLPRGEPPPGRLK
SAPLFMLDLYNALSADNDEDGASEGERQQSWPHEAASSSQRRQPPPGAAHPLNRKSLLAPGSGSG
GASPLTSAQDSAFLNDADMVMSFVNLVEYDKEFSPRQRHHKEFKFNLSQIPEGEVVTAAEFRIYK
DCVMGSFKNQTFLISIYQVLQEHQHRDSDLFLLDTRVVWASEEGWLEFDITATSNLWVVTPQHNM
GLQLSVVTRDGVHVHPRAAGLVGRDGPYDKQPFMVAFFKVSEVHVRTTRSASSRRRQQSRNRSTQ
SQDVARVSSASDYNSSELKTACRKHELYVSFQDLGWQDWIIAPKGYAANYCDGECSFPLNAHMNA
TNHAIVQTLVHLMNPEYVPKPCCAPTKLNAISVLYFDDNSNVILKKYRNMVVRACGCH*
SEQ ID NO: 18:
amino acid sequence of BMP6 with signal peptide
CCGPPPLRPPLPAAAAAAAGGQLLGDGGSPGRTEQPPPSPQSSSGFLYRRLKTQEKREMQKEILS
VLGLPHRPRPLHGLQQPQPPALRQQEEQQQQQQLPRGEPPPGRLKSAPLFMLDLYNALSADNDED
GASEGERQQSWPHEAASSSQRRQPPPGAAHPLNRKSLLAPGSGSGGASPLTSAQDSAFLNDADMV
MSFVNLVEYDKEFSPRQRHHKEFKFNLSQIPEGEVVTAAEFRIYKDCVMGSFKNQTFLISIYQVL
QEHQHRDSDLFLLDTRVVWASEEGWLEFDITATSNLWVVTPQHNMGLQLSVVTRDGVHVHPRAAG
LVGRDGPYDKQPFMVAFFKVSEVHVRTTRSASSRRRQQSRNRSTQSQDVARVSSASDYNSSELKT
ACRKHELYVSFQDLGWQDWIIAPKGYAANYCDGECSFPLNAHMNATNHAIVQTLVHLMNPEYVPK
PCCAPTKLNAISVLYFDDNSNVILKKYRNMVVRACGCH*
SEQ ID NO: 19:
IGF1 optimized mRNA without signal peptide
GTGAAGATGCACACCATGAGCAGCAGCCACCTCTTTTACCTGGCCCTGTGCCTGCTGACCTTCAC
CAGCAGCGCTACAGCCGGACCTGAGACCCTGTGTGGCGCCGAGCTGGTCGATGCCCTGCAGTTCG
TGTGCGGAGACAGAGGCTTCTATTTCAACAAGCCTACAGGCTACGGCTCTAGCTCCAGAAGAGCC
CCACAGACCGGCATCGTGGACGAGTGCTGTTTTAGAAGCTGTGATCTGAGAAGGCTGGAAATGTA
CTGCGCCCCTCTGAAACCCGCTAAGTCAGCCAGATCCGTGCGGGCCCAGCGGCACACAGACATGC
CTAAGACCCAAAAGGAAGTGCATCTGAAGAATGCCTCTAGAGGATCTGCTGGAAACAAGAACTAC
CGGATGTGA
SEQ ID NO: 20:
IGF1 optimized mRNA with signal peptide
ATGGGCAAGATCAGCAGCCTGCCCACACAGCTGTTCAAGTGCTGCTTCTGCGACTTCCTGAAGGT
GAAGATGCACACCATGAGCAGCAGCCACCTCTTTTACCTGGCCCTGTGCCTGCTGACCTTCACCA
GCAGCGCTACAGCCGGACCTGAGACCCTGTGTGGCGCCGAGCTGGTCGATGCCCTGCAGTTCGTG
TGCGGAGACAGAGGCTTCTATTTCAACAAGCCTACAGGCTACGGCTCTAGCTCCAGAAGAGCCCC
ACAGACCGGCATCGTGGACGAGTGCTGTTTTAGAAGCTGTGATCTGAGAAGGCTGGAAATGTACT
GCGCCCCTCTGAAACCCGCTAAGTCAGCCAGATCCGTGCGGGCCCAGCGGCACACAGACATGCCT
AAGACCCAAAAGGAAGTGCATCTGAAGAATGCCTCTAGAGGATCTGCTGGAAACAAGAACTACCG
GATGTGA
SEQ ID NO: 21:
IGF1 mRNA without signal peptide
GTGAAGATGCACACCATGTCCTCCTCGCATCTCTTCTACCTGGCGCTGTGCCTGCTCACCTTCAC
CAGCTCTGCCACGGCTGGACCGGAGACGCTCTGCGGGGCTGAGCTGGTGGATGCTCTTCAGTTCG
TGTGTGGAGACAGGGGCTTTTATTTCAACAAGCCCACAGGGTATGGCTCCAGCAGTCGGAGGGCG
CCTCAGACAGGCATCGTGGATGAGTGCTGCTTCCGGAGCTGTGATCTAAGGAGGCTGGAGATGTA
TTGCGCACCCCTCAAGCCTGCCAAGTCAGCTCGCTCTGTCCGTGCCCAGCGCCACACCGACATGC
CCAAGACCCAGAAGGAAGTACATTTGAAGAACGCAAGTAGAGGGAGTGCAGGAAACAAGAACTAC
AGGATGTAG
SEQ ID NO: 22:
IGF1 mRNA with signal peptide
ATGGGAAAAATCAGCAGTCTTCCAACCCAATTATTTAAGTGCTGCTTTTGTGATTTCTTGAAGGT
GAAGATGCACACCATGTCCTCCTCGCATCTCTTCTACCTGGCGCTGTGCCTGCTCACCTTCACCA
GCTCTGCCACGGCTGGACCGGAGACGCTCTGCGGGGCTGAGCTGGTGGATGCTCTTCAGTTCGTG
TGTGGAGACAGGGGCTTTTATTTCAACAAGCCCACAGGGTATGGCTCCAGCAGTCGGAGGGCGCC
TCAGACAGGCATCGTGGATGAGTGCTGCTTCCGGAGCTGTGATCTAAGGAGGCTGGAGATGTATT
GCGCACCCCTCAAGCCTGCCAAGTCAGCTCGCTCTGTCCGTGCCCAGCGCCACACCGACATGCCC
AAGACCCAGAAGGAAGTACATTTGAAGAACGCAAGTAGAGGGAGTGCAGGAAACAAGAACTACAG
GATGTAG
SEQ ID NO: 23:
amino acid sequence of IGF1 without signal peptide
VKMHTMSSSHLFYLALCLLTFTSSATAGPETLCGAELVDALQFVCGDRGFYFNKPTGYGSSSRRA
PQTGIVDECCFRSCDLRRLEMYCAPLKPAKSARSVRAQRHTDMPKTQKEVHLKNASRGSAGNKNY
RM*
SEQ ID NO: 24:
amino acid sequence of IGF1 with signal peptide
MGKISSLPTQLFKCCFCDFLKVKMHTMSSSHLFYLALCLLTFTSSATAGPETLCGAELVDALQFV
CGDRGFYFNKPTGYGSSSRRAPQTGIVDECCFRSCDLRRLEMYCAPLKPAKSARSVRAQRHTDMP
KTQKEVHLKNASRGSAGNKNYRM*
SEQ ID NO: 25:
TGFβ3 optimized mRNA without signal peptide
CTGTCTACATGCACCACCCTGGATTTCGGCCACATCAAGAAAAAACGGGTCGAGGCCATCAGAGG
TCAGATCCTGAGCAAGCTGAGGCTGACCAGCCCTCCAGAGCCTACCGTGATGACCCACGTGCCTT
ACCAGGTGCTGGCCCTGTATAATTCTACAAGAGAGCTGCTGGAAGAGATGCACGGAGAAAGAGAG
GAAGGCTGTACCCAGGAGAATACCGAGAGCGAGTACTACGCCAAGGAAATCCACAAGTTCGACAT
GATCCAGGGCCTGGCTGAACATAACGAGCTGGCCGTGTGCCCTAAGGGCATCACCAGCAAGGTGT
TCAGATTCAACGTGTCCAGCGTGGAAAAGAACAGAACAAACCTGTTTCGGGCCGAGTTCCGGGTG
CTGAGAGTGCCCAATCCTTCTTCCAAGCGGAACGAGCAGAGAATCGAACTGTTCCAAATCCTGAG
ACCCGATGAGCACATCGCTAAACAGAGATACATCGGCGGCAAGAACCTGCCCACACGGGGCACCG
CCGAGTGGCTGAGCTTTGATGTGACCGACACCGTGAGAGAGTGGCTGCTGCGGCGGGAGAGCAAC
CTGGGCCTCGAAATCAGCATCCACTGCCCTTGCCACACATTCCAGCCTAACGGCGACATCCTGGA
AAACATCCACGAGGTGATGGAAATTAAGTTTAAGGGCGTGGACAACGAGGACGACCACGGCCGCG
GCGATCTGGGCAGACTGAAGAAGCAGAAGGACCACCACAACCCCCACCTGATTCTCATGATGATC
CCTCCACACAGACTGGATAACCCTGGACAGGGCGGACAAAGAAAGAAGAGAGCCCTGGACACCAA
CTACTGCTTCCGGAACCTGGAAGAGAACTGCTGCGTGCGGCCACTGTACATCGACTTCAGACAGG
ATCTGGGATGGAAGTGGGTGCACGAGCCCAAAGGCTACTACGCTAACTTCTGCTCAGGCCCTTGC
CCTTACCTGAGAAGCGCCGACACAACCCACAGCACAGTGCTGGGACTGTACAACACCCTGAACCC
AGAGGCCAGCGCCAGCCCCTGTTGTGTGCCACAGGACCTCGAGCCTCTGACAATCCTGTACTATG
TGGGCAGAACCCCTAAGGTCGAACAGCTGTCTAATATGGTGGTGAAGTCTTGTAAATGCAGCTGA
SEQ ID NO: 26:
TGFβ3 optimized mRNA with signal peptide
ATGAAAATGCACCTGCAGAGAGCTCTGGTCGTGCTGGCCCTGCTGAACTTCGCCACCGTATCCCT
GAGCCTGTCTACATGCACCACCCTGGATTTCGGCCACATCAAGAAAAAACGGGTCGAGGCCATCA
GAGGTCAGATCCTGAGCAAGCTGAGGCTGACCAGCCCTCCAGAGCCTACCGTGATGACCCACGTG
CCTTACCAGGTGCTGGCCCTGTATAATTCTACAAGAGAGCTGCTGGAAGAGATGCACGGAGAAAG
AGAGGAAGGCTGTACCCAGGAGAATACCGAGAGCGAGTACTACGCCAAGGAAATCCACAAGTTCG
ACATGATCCAGGGCCTGGCTGAACATAACGAGCTGGCCGTGTGCCCTAAGGGCATCACCAGCAAG
GTGTTCAGATTCAACGTGTCCAGCGTGGAAAAGAACAGAACAAACCTGTTTCGGGCCGAGTTCCG
GGTGCTGAGAGTGCCCAATCCTTCTTCCAAGCGGAACGAGCAGAGAATCGAACTGTTCCAAATCC
TGAGACCCGATGAGCACATCGCTAAACAGAGATACATCGGCGGCAAGAACCTGCCCACACGGGGC
ACCGCCGAGTGGCTGAGCTTTGATGTGACCGACACCGTGAGAGAGTGGCTGCTGCGGCGGGAGAG
CAACCTGGGCCTCGAAATCAGCATCCACTGCCCTTGCCACACATTCCAGCCTAACGGCGACATCC
TGGAAAACATCCACGAGGTGATGGAAATTAAGTTTAAGGGCGTGGACAACGAGGACGACCACGGC
CGCGGCGATCTGGGCAGACTGAAGAAGCAGAAGGACCACCACAACCCCCACCTGATTCTCATGAT
GATCCCTCCACACAGACTGGATAACCCTGGACAGGGCGGACAAAGAAAGAAGAGAGCCCTGGACA
CCAACTACTGCTTCCGGAACCTGGAAGAGAACTGCTGCGTGCGGCCACTGTACATCGACTTCAGA
CAGGATCTGGGATGGAAGTGGGTGCACGAGCCCAAAGGCTACTACGCTAACTTCTGCTCAGGCCC
TTGCCCTTACCTGAGAAGCGCCGACACAACCCACAGCACAGTGCTGGGACTGTACAACACCCTGA
ACCCAGAGGCCAGCGCCAGCCCCTGTTGTGTGCCACAGGACCTCGAGCCTCTGACAATCCTGTAC
TATGTGGGCAGAACCCCTAAGGTCGAACAGCTGTCTAATATGGTGGTGAAGTCTTGTAAATGCAG
CTGA
SEQ ID NO: 27:
TGFβ3 mRNA without signal peptide
CTGTCCACTTGCACCACCTTGGACTTCGGCCACATCAAGAAGAAGAGGGTGGAAGCCATTAGGGG
ACAGATCTTGAGCAAGCTCAGGCTCACCAGCCCCCCTGAGCCAACGGTGATGACCCACGTCCCCT
ATCAGGTCCTGGCCCTTTACAACAGCACCCGGGAGCTGCTGGAGGAGATGCATGGGGAGAGGGAG
GAAGGCTGCACCCAGGAAAACACCGAGTCGGAATACTATGCCAAAGAAATCCATAAATTCGACAT
GATCCAGGGGCTGGCGGAGCACAACGAACTGGCTGTCTGCCCTAAAGGAATTACCTCCAAGGTTT
TCCGCTTCAATGTGTCCTCAGTGGAGAAAAATAGAACCAACCTATTCCGAGCAGAATTCCGGGTC
TTGCGGGTGCCCAACCCCAGCTCTAAGCGGAATGAGCAGAGGATCGAGCTCTTCCAGATCCTTCG
GCCAGATGAGCACATTGCCAAACAGCGCTATATCGGTGGCAAGAATCTGCCCACACGGGGCACTG
CCGAGTGGCTGTCCTTTGATGTCACTGACACTGTGCGTGAGTGGCTGTTGAGAAGAGAGTCCAAC
TTAGGTCTAGAAATCAGCATTCACTGTCCATGTCACACCTTTCAGCCCAATGGAGATATCCTGGA
AAACATTCACGAGGTGATGGAAATCAAATTCAAAGGCGTGGACAATGAGGATGACCATGGCCGTG
GAGATCTGGGGCGCCTCAAGAAGCAGAAGGATCACCACAACCCTCATCTAATCCTCATGATGATT
CCCCCACACCGGCTCGACAACCCGGGCCAGGGGGGTCAGAGGAAGAAGCGGGCTTTGGACACCAA
TTACTGCTTCCGCAACTTGGAGGAGAACTGCTGTGTGCGCCCCCTCTACATTGACTTCCGACAGG
ATCTGGGCTGGAAGTGGGTCCATGAACCTAAGGGCTACTATGCCAACTTCTGCTCAGGCCCTTGC
CCATACCTCCGCAGTGCAGACACAACCCACAGCACGGTGCTGGGACTGTACAACACTCTGAACCC
TGAAGCATCTGCCTCGCCTTGCTGCGTGCCCCAGGACCTGGAGCCCCTGACCATCCTGTACTATG
TTGGGAGGACCCCCAAAGTGGAGCAGCTCTCCAACATGGTGGTGAAGTCTTGTAAATGTAGCTGA
SEQ ID NO: 28:
TGFβ3 mRNA with signal peptide
ATGAAGATGCACTTGCAAAGGGCTCTGGTGGTCCTGGCCCTGCTGAACTTTGCCACGGTCAGCCT
CTCTCTGTCCACTTGCACCACCTTGGACTTCGGCCACATCAAGAAGAAGAGGGTGGAAGCCATTA
GGGGACAGATCTTGAGCAAGCTCAGGCTCACCAGCCCCCCTGAGCCAACGGTGATGACCCACGTC
CCCTATCAGGTCCTGGCCCTTTACAACAGCACCCGGGAGCTGCTGGAGGAGATGCATGGGGAGAG
GGAGGAAGGCTGCACCCAGGAAAACACCGAGTCGGAATACTATGCCAAAGAAATCCATAAATTCG
ACATGATCCAGGGGCTGGCGGAGCACAACGAACTGGCTGTCTGCCCTAAAGGAATTACCTCCAAG
GTTTTCCGCTTCAATGTGTCCTCAGTGGAGAAAAATAGAACCAACCTATTCCGAGCAGAATTCCG
GGTCTTGCGGGTGCCCAACCCCAGCTCTAAGCGGAATGAGCAGAGGATCGAGCTCTTCCAGATCC
TTCGGCCAGATGAGCACATTGCCAAACAGCGCTATATCGGTGGCAAGAATCTGCCCACACGGGGC
ACTGCCGAGTGGCTGTCCTTTGATGTCACTGACACTGTGCGTGAGTGGCTGTTGAGAAGAGAGTC
CAACTTAGGTCTAGAAATCAGCATTCACTGTCCATGTCACACCTTTCAGCCCAATGGAGATATCC
TGGAAAACATTCACGAGGTGATGGAAATCAAATTCAAAGGCGTGGACAATGAGGATGACCATGGC
CGTGGAGATCTGGGGCGCCTCAAGAAGCAGAAGGATCACCACAACCCTCATCTAATCCTCATGAT
GATTCCCCCACACCGGCTCGACAACCCGGGCCAGGGGGGTCAGAGGAAGAAGCGGGCTTTGGACA
CCAATTACTGCTTCCGCAACTTGGAGGAGAACTGCTGTGTGCGCCCCCTCTACATTGACTTCCGA
CAGGATCTGGGCTGGAAGTGGGTCCATGAACCTAAGGGCTACTATGCCAACTTCTGCTCAGGCCC
TTGCCCATACCTCCGCAGTGCAGACACAACCCACAGCACGGTGCTGGGACTGTACAACACTCTGA
ACCCTGAAGCATCTGCCTCGCCTTGCTGCGTGCCCCAGGACCTGGAGCCCCTGACCATCCTGTAC
TATGTTGGGAGGACCCCCAAAGTGGAGCAGCTCTCCAACATGGTGGTGAAGTCTTGTAAATGTAG
CTGA
SEQ ID NO: 29:
amino acid sequence of TGFβ3 without signal peptide
LSTCTTLDFGHIKKKRVEAIRGQILSKLRLTSPPEPTVMTHVPYQVLALYNSTRELLEEMHGERE
EGCTQENTESEYYAKEIHKFDMIQGLAEHNELAVCPKGITSKVFRFNVSSVEKNRTNLFRAEFRV
LRVPNPSSKRNEQRIELFQILRPDEHIAKQRYIGGKNLPTRGTAEWLSFDVTDTVREWLLRRESN
LGLEISIHCPCHTFQPNGDILENIHEVMEIKFKGVDNEDDHGRGDLGRLKKQKDHHNPHLILMMI
PPHRLDNPGQGGQRKKRALDTNYCFRNLEENCCVRPLYIDFRQDLGWKWVHEPKGYYANFCSGPC
PYLRSADTTHSTVLGLYNTLNPEASASPCCVPQDLEPLTILYYVGRTPKVEQLSNMVVKSCKCS*
SEQ ID NO: 30:
amino acid sequence of TGFβ3 with signal peptide
MKMHLQRALVVLALLNFATVSLSLSTCTTLDFGHIKKKRVEAIRGQILSKLRLTSPPEPTVMTHV
PYQVLALYNSTRELLEEMHGEREEGCTQENTESEYYAKEIHKFDMIQGLAEHNELAVCPKGITSK
VFRFNVSSVEKNRTNLFRAEFRVLRVPNPSSKRNEQRIELFQILRPDEHIAKQRYIGGKNLPTRG
TAEWLSFDVTDTVREWLLRRESNLGLEISIHCPCHTFQPNGDILENIHEVMEIKFKGVDNEDDHG
RGDLGRLKKQKDHHNPHLILMMIPPHRLDNPGQGGQRKKRALDTNYCFRNLEENCCVRPLYIDFR
QDLGWKWVHEPKGYYANFCSGPCPYLRSADTTHSTVLGLYNTLNPEASASPCCVPQDLEPLTILY
YVGRTPKVEQLSNMVVKSCKCS*
SEQ ID NO: 31:
WNT3a optimized mRNA without signal peptide
AGCTACCCCATCTGGTGGTCCCTCGCTGTGGGCCCTCAGTACAGCTCTCTGGGATCTCAGCCTAT
CCTGTGCGCCAGCATCCCTGGCCTGGTCCCCAAGCAGCTGAGATTCTGCAGAAACTACGTGGAAA
TCATGCCCAGCGTGGCAGAAGGCATCAAGATCGGAATTCAGGAGTGCCAACATCAATTCAGAGGC
CGGCGCTGGAACTGCACAACAGTGCACGACAGCCTGGCCATCTTCGGCCCTGTGCTGGATAAGGC
CACCAGGGAGTCAGCTTTCGTGCACGCAATCGCATCAGCAGGTGTAGCCTTTGCGGTGACCAGAA
GCTGTGCAGAGGGCACAGCCGCTATCTGTGGCTGTAGCAGCCGGCACCAGGGCAGCCCCGGCAAG
GGCTGGAAGTGGGGAGGTTGTAGCGAGGACATCGAGTTCGGCGGCATGGTTTCTAGAGAATTCGC
CGACGCTAGAGAGAACCGGCCAGATGCCCGCAGCGCCATGAACAGACACAACAACGAGGCCGGAC
GGCAGGCCATCGCCTCTCATATGCACCTGAAGTGCAAGTGCCACGGCCTGTCTGGCAGCTGCGAG
GTGAAGACCTGCTGGTGGAGCCAGCCTGATTTTCGGGCCATCGGCGACTTCCTGAAAGACAAGTA
CGACAGCGCCAGCGAGATGGTCGTGGAGAAGCACAGAGAAAGCAGAGGATGGGTGGAAACCCTGC
GGCCTAGATACACCTACTTCAAGGTGCCTACCGAGCGGGACCTGGTGTACTACGAGGCCAGCCCT
AATTTCTGTGAACCCAATCCAGAGACCGGCAGCTTCGGAACAAGAGATAGAACATGCAACGTGTC
CAGCCACGGCATCGACGGCTGCGACCTGCTTTGCTGCGGCAGAGGCCACAACGCCAGAGCTGAAA
GAAGACGGGAAAAATGTAGATGCGTGTTCCACTGGTGCTGCTACGTGTCCTGCCAGGAATGTACA
CGGGTGTATGATGTGCACACCTGCAAATGA
SEQ ID NO: 32:
WNT3a optimized mRNA with signal peptide
ATGGCCCCTCTGGGCTACTTTCTGCTGCTGTGCTCCCTGAAGCAGGCCCTGGGCAGCTACCCCAT
CTGGTGGTCCCTCGCTGTGGGCCCTCAGTACAGCTCTCTGGGATCTCAGCCTATCCTGTGCGCCA
GCATCCCTGGCCTGGTCCCCAAGCAGCTGAGATTCTGCAGAAACTACGTGGAAATCATGCCCAGC
GTGGCAGAAGGCATCAAGATCGGAATTCAGGAGTGCCAACATCAATTCAGAGGCCGGCGCTGGAA
CTGCACAACAGTGCACGACAGCCTGGCCATCTTCGGCCCTGTGCTGGATAAGGCCACCAGGGAGT
CAGCTTTCGTGCACGCAATCGCATCAGCAGGTGTAGCCTTTGCGGTGACCAGAAGCTGTGCAGAG
GGCACAGCCGCTATCTGTGGCTGTAGCAGCCGGCACCAGGGCAGCCCCGGCAAGGGCTGGAAGTG
GGGAGGTTGTAGCGAGGACATCGAGTTCGGCGGCATGGTTTCTAGAGAATTCGCCGACGCTAGAG
AGAACCGGCCAGATGCCCGCAGCGCCATGAACAGACACAACAACGAGGCCGGACGGCAGGCCATC
GCCTCTCATATGCACCTGAAGTGCAAGTGCCACGGCCTGTCTGGCAGCTGCGAGGTGAAGACCTG
CTGGTGGAGCCAGCCTGATTTTCGGGCCATCGGCGACTTCCTGAAAGACAAGTACGACAGCGCCA
GCGAGATGGTCGTGGAGAAGCACAGAGAAAGCAGAGGATGGGTGGAAACCCTGCGGCCTAGATAC
ACCTACTTCAAGGTGCCTACCGAGCGGGACCTGGTGTACTACGAGGCCAGCCCTAATTTCTGTGA
ACCCAATCCAGAGACCGGCAGCTTCGGAACAAGAGATAGAACATGCAACGTGTCCAGCCACGGCA
TCGACGGCTGCGACCTGCTTTGCTGCGGCAGAGGCCACAACGCCAGAGCTGAAAGAAGACGGGAA
AAATGTAGATGCGTGTTCCACTGGTGCTGCTACGTGTCCTGCCAGGAATGTACACGGGTGTATGA
TGTGCACACCTGCAAATGA
SEQ ID NO: 33:
WNT3a mRNA without signal peptide
AGCTACCCGATCTGGTGGTCGCTGGCTGTTGGGCCACAGTATTCCTCCCTGGGCTCGCAGCCCAT
CCTGTGTGCCAGCATCCCGGGCCTGGTCCCCAAGCAGCTCCGCTTCTGCAGGAACTACGTGGAGA
TCATGCCCAGCGTGGCCGAGGGCATCAAGATTGGCATCCAGGAGTGCCAGCACCAGTTCCGCGGC
CGCCGGTGGAACTGCACCACCGTCCACGACAGCCTGGCCATCTTCGGGCCCGTGCTGGACAAAGC
TACCAGGGAGTCGGCCTTTGTCCACGCCATTGCCTCAGCCGGTGTGGCCTTTGCAGTGACACGCT
CATGTGCAGAAGGCACGGCCGCCATCTGTGGCTGCAGCAGCCGCCACCAGGGCTCACCAGGCAAG
GGCTGGAAGTGGGGTGGCTGTAGCGAGGACATCGAGTTTGGTGGGATGGTGTCTCGGGAGTTCGC
CGACGCCCGGGAGAACCGGCCAGATGCCCGCTCAGCCATGAACCGCCACAACAACGAGGCTGGGC
GCCAGGCCATCGCCAGCCACATGCACCTCAAGTGCAAGTGCCACGGGCTGTCGGGCAGCTGCGAG
GTGAAGACATGCTGGTGGTCGCAACCCGACTTCCGCGCCATCGGTGACTTCCTCAAGGACAAGTA
CGACAGCGCCTCGGAGATGGTGGTGGAGAAGCACCGGGAGTCCCGCGGCTGGGTGGAGACCCTGC
GGCCGCGCTACACCTACTTCAAGGTGCCCACGGAGCGCGACCTGGTCTACTACGAGGCCTCGCCC
AACTTCTGCGAGCCCAACCCTGAGACGGGCTCCTTCGGCACGCGCGACCGCACCTGCAACGTCAG
CTCGCACGGCATCGACGGCTGCGACCTGCTGTGCTGCGGCCGCGGCCACAACGCGCGAGCGGAGC
GGCGCCGGGAGAAGTGCCGCTGCGTGTTCCACTGGTGCTGCTACGTCAGCTGCCAGGAGTGCACG
CGCGTCTACGACGTGCACACCTGCAAGTAG
SEQ ID NO: 34:
WNT3a mRNA with signal peptide
ATGGCCCCACTCGGATACTTCTTACTCCTCTGCAGCCTGAAGCAGGCTCTGGGCAGCTACCCGAT
CTGGTGGTCGCTGGCTGTTGGGCCACAGTATTCCTCCCTGGGCTCGCAGCCCATCCTGTGTGCCA
GCATCCCGGGCCTGGTCCCCAAGCAGCTCCGCTTCTGCAGGAACTACGTGGAGATCATGCCCAGC
GTGGCCGAGGGCATCAAGATTGGCATCCAGGAGTGCCAGCACCAGTTCCGCGGCCGCCGGTGGAA
CTGCACCACCGTCCACGACAGCCTGGCCATCTTCGGGCCCGTGCTGGACAAAGCTACCAGGGAGT
CGGCCTTTGTCCACGCCATTGCCTCAGCCGGTGTGGCCTTTGCAGTGACACGCTCATGTGCAGAA
GGCACGGCCGCCATCTGTGGCTGCAGCAGCCGCCACCAGGGCTCACCAGGCAAGGGCTGGAAGTG
GGGTGGCTGTAGCGAGGACATCGAGTTTGGTGGGATGGTGTCTCGGGAGTTCGCCGACGCCCGGG
AGAACCGGCCAGATGCCCGCTCAGCCATGAACCGCCACAACAACGAGGCTGGGCGCCAGGCCATC
GCCAGCCACATGCACCTCAAGTGCAAGTGCCACGGGCTGTCGGGCAGCTGCGAGGTGAAGACATG
CTGGTGGTCGCAACCCGACTTCCGCGCCATCGGTGACTTCCTCAAGGACAAGTACGACAGCGCCT
CGGAGATGGTGGTGGAGAAGCACCGGGAGTCCCGCGGCTGGGTGGAGACCCTGCGGCCGCGCTAC
ACCTACTTCAAGGTGCCCACGGAGCGCGACCTGGTCTACTACGAGGCCTCGCCCAACTTCTGCGA
GCCCAACCCTGAGACGGGCTCCTTCGGCACGCGCGACCGCACCTGCAACGTCAGCTCGCACGGCA
TCGACGGCTGCGACCTGCTGTGCTGCGGCCGCGGCCACAACGCGCGAGCGGAGCGGCGCCGGGAG
AAGTGCCGCTGCGTGTTCCACTGGTGCTGCTACGTCAGCTGCCAGGAGTGCACGCGCGTCTACGA
CGTGCACACCTGCAAGTAG
SEQ ID NO: 35:
amino acid sequence of WNT3a without signal peptide
SYPIWWSLAVGPQYSSLGSQPILCASIPGLVPKQLRFCRNYVEIMPSVAEGIKIGIQECQHQFRG
RRWNCTTVHDSLAIFGPVLDKATRESAFVHAIASAGVAFAVTRSCAEGTAAICGCSSRHQGSPGK
GWKWGGCSEDIEFGGMVSREFADARENRPDARSAMNRHNNEAGRQAIASHMHLKCKCHGLSGSCE
VKTCWWSQPDFRAIGDFLKDKYDSASEMVVEKHRESRGWVETLRPRYTYFKVPTERDLVYYEASP
NFCEPNPETGSFGTRDRTCNVSSHGIDGCDLLCCGRGHNARAERRREKCRCVFHWCCYVSCQECT
RVYDVHTCK*
SEQ ID NO: 36:
amino acid sequence of WNT3a with signal peptide
MAPLGYFLLLCSLKQALGSYPIWWSLAVGPQYSSLGSQPILCASIPGLVPKQLRFCRNYVEIMPS
VAEGIKIGIQECQHQFRGRRWNCTTVHDSLAIFGPVLDKATRESAFVHAIASAGVAFAVTRSCAE
GTAAICGCSSRHQGSPGKGWKWGGCSEDIEFGGMVSREFADARENRPDARSAMNRHNNEAGRQAI
ASHMHLKCKCHGLSGSCEVKTCWWSQPDFRAIGDFLKDKYDSASEMVVEKHRESRGWVETLRPRY
TYFKVPTERDLVYYEASPNFCEPNPETGSFGTRDRTCNVSSHGIDGCDLLCCGRGHNARAERRRE
KCRCVFHWCCYVSCQECTRVYDVHTCK*

### REFERENCES

Abramson S B, Attur M. Developments in the scientific understanding of osteoarthritis. Arthritis Res Ther. 2009; 11(3):227. doi: 10.1186/ar2655. Epub 2009 May 19.
Chaplan S R, Bach F W, Pogrel J W, Chung J M, Yaksh T L. Quantitative assessment of tactile allodynia in the rat paw. J Neurosci Methods. 1994 July; 53(1):55-63. doi: 10.1016/0165-0270(94)90144-9.
Filippakopoulos P, Qi J, Picaud S, Shen Y, Smith W B, Fedorov O, Morse E M, Keates T, Hickman T T, Felletar I, Philpott M, Munro S, Mckeown M R, Wang Y, Christie A L, West N, Cameron M J, Schwartz B, Heightman T D, La Thangue N, French C A, Wiest O, Kung A L, Knapp S, Bradner J E. Selective inhibition of BET bromodomains. Nature. 2010 Dec. 23; 468(7327):1067-73. doi: 10.1038/nature09504. Epub 2010 Sep. 24.
Fitzsimmons R E B, Mazurek M S, Soos A, Simmons C A. Mesenchymal Stromal/Stem Cells in Regenerative Medicine and Tissue Engineering. Stem Cells Int. 2018 Aug. 19; 2018:8031718. doi: 10.1155/2018/8031718.
Gerwin N, Bendele A M, Glasson S, Carlson C S. The OARSI histopathology initiative-recommendations for histological assessments of osteoarthritis in the rat. Osteoarthritis Cartilage. 2010 October; 18 Suppl 3:S24-34. doi: 10.1016/j.joca.2010.05.030. PMID: 20864021.
Soontararak S, Chow L, Johnson V, Coy J, Wheat W, Regan D, Dow S. Mesenchymal Stem Cells (MSC) Derived from Induced Pluripotent Stem Cells (iPSC) Equivalent to Adipose-Derived MSC in Promoting Intestinal Healing and Microbiome Normalization in Mouse Inflammatory Bowel Disease Model. Stem Cells Transl Med. 2018 June; 7(6):456-467. doi: 10.1002/sctm.17-0305. Epub 2018 Apr. 10.
Ullah I, Subbarao R B, Rho G J. Human mesenchymal stem cells—current trends and future prospective. Biosci Rep. 2015 Apr. 28; 35(2):e00191. doi: 10.1042/BSR20150025.
Xu M, Shaw G, Murphy M, Barry F. Induced Pluripotent Stem Cell-Derived Mesenchymal Stromal Cells Are Functionally and Genetically Different From Bone Marrow-Derived Mesenchymal Stromal Cells. Stem Cells. 2019 June; 37(6):754-765. doi: 10.1002/stem.2993. Epub 2019 Mar. 6.
Yang S, Xie C, Chen Y, Wang J, Chen X, Lu Z, June R R, Zheng S G. Differential roles of TNFα-TNFR1 and TNFα-TNFR2 in the differentiation and function of CD4+Foxp3+ induced Treg cells in vitro and in vivo periphery in autoimmune diseases. Cell Death Dis. 2019 Jan. 10; 10(1):27. doi: 10.1038/s41419-018-1266-6.
Zhu C, Wu W, Qu X. Mesenchymal stem cells in osteoarthritis therapy: a review. Am J Transl Res. 2021 Feb. 15; 13(2):448-461.

### Items

1. Mesenchymal stem cells (MSCs) for use in a method of treatment of arthritis, wherein the MSCs are transfected with an mRNA construct encoding a pro-chondrogenic and/or chondrocyte protective factor.
2. Mesenchymal stem cells (MSCs), wherein the MSCs are transfected with an mRNA construct encoding a pro-chondrogenic and/or chondrocyte protective factor.
3. The MSCs or the MSCs for use in a method of treatment of arthritis according to any one of the preceding items, wherein the mRNA construct comprises mRNA encoding an TGFβ3 of SEQ ID NO: 29 or a functional homologue of said TGFβ3 sharing at least 70%, such as at least 80%, for example at least 90%, such as at least 95% sequence identity therewith.
4. The MSCs or the MSCs for use in a method of treatment of arthritis according to any one of the preceding items, wherein the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 25.
5. The MSCs or the MSCs for use in a method of treatment of arthritis according to any one of the preceding items, wherein the mRNA construct comprises mRNA encoding TGFβ3 of SEQ ID NO: 29 or a functional homologue of said TGFβ3 sharing at least 70%, such as at least 80%, for example at least 90%, such as at least 95% sequence identity therewith and a signal peptide.
6. The MSCs or the MSCs for use in a method of treatment of arthritis according to any one of the preceding items, wherein the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 25 and a signal peptide.
7. The MSCs or the MSCs for use in a method of treatment of arthritis according to any one of items 4 to 5, wherein the signal peptide is a heterologous or native signal peptide.
8. The MSCs or the MSCs for use in a method of treatment of arthritis according to any one of the preceding items, wherein the mRNA construct encodes an TGFβ3 of SEQ ID NO: 30 or a functional homologue of said TGFβ3 sharing at least 70%, such as at least 80%, for example at least 90%, such as at least 95% sequence identity therewith.
9. The MSCs or the MSCs for use in a method of treatment of arthritis according to any one of the preceding items, wherein the mRNA construct comprises an mRNA construct having at least 70%, such as at least 80%, such as at least 85%, such as at least 90%, such as at least 95%, such as at least 97%, such as at least 99%, such as 100% sequence identity to SEQ ID NO: 26.
10. The MSCs or the MSCs for use in a method of treatment of arthritis according to any one of the preceding items, wherein the mRNA construct comprises mRNA encoding an IGF1 of SEQ ID NO: 23 or a functional homologue of said IGF1 sharing at least 70%, such as at least 80%, for example at least 90%, such as at least 95% sequence identity therewith.
11. The MSCs or the MSCs for use in a method of treatment of arthritis according to any one of the preceding items, wherein the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 19.
12. The MSCs or the MSCs for use in a method of treatment of arthritis according to any one of the preceding items, wherein the mRNA construct comprises mRNA encoding IGF1 of SEQ ID NO: 23 or a functional homologue of said IGF1 sharing at least 70%, such as at least 80%, for example at least 90%, such as at least 95% sequence identity therewith and a signal peptide.
13. The MSCs or the MSCs for use in a method of treatment of arthritis according to any one of the preceding items, wherein the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 19 and a signal peptide.
14. The MSCs or the MSCs for use in a method of treatment of arthritis according to any one of the preceding items, wherein the mRNA construct encodes an IGF1 of SEQ ID NO: 24 or a functional homologue of said IGF1 sharing at least 70%, such as at least 80%, for example at least 90%, such as at least 95% sequence identity therewith.
15. The MSCs or the MSCs for use in a method of treatment of arthritis according to any one of the preceding items, wherein the mRNA construct comprises an mRNA construct having at least 70%, such as at least 80%, such as at least 85%, such as at least 90%, such as at least 95%, such as at least 97%, such as at least 99%, such as 100% sequence identity to SEQ ID NO: 20.
16. The MSCs or the MSCs for use in a method of treatment of arthritis according to any one of the preceding items, wherein the mRNA construct comprises mRNA encoding an BMP6 of SEQ ID NO: 17 or a functional homologue of said BMP6 sharing at least 70%, such as at least 80%, for example at least 90%, such as at least 95% sequence identity therewith.
17. The MSCs or the MSCs for use in a method of treatment of arthritis according to any one of the preceding items, wherein the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 13.
18. The MSCs or the MSCs for use in a method of treatment of arthritis according to any one of the preceding items, wherein the mRNA construct comprises mRNA encoding BMP6 of SEQ ID NO: 17 or a functional homologue of said BMP6 sharing at least 70%, such as at least 80%, for example at least 90%, such as at least 95% sequence identity therewith and a signal peptide.
19. The MSCs or the MSCs for use in a method of treatment of arthritis according to any one of the preceding items, wherein the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 13 and a signal peptide.
20. The MSCs or the MSCs for use in a method of treatment of arthritis according to any one of the preceding items, wherein the mRNA construct encodes an BMP6 of SEQ ID NO: 18 or a functional homologue of said BMP6 sharing at least 70%, such as at least therewith.
21. The MSCs or the MSCs for use in a method of treatment of arthritis according to any one of the preceding items, wherein the mRNA construct comprises an mRNA construct having at least 70%, such as at least 80%, such as at least 85%, such as at least 90%, such as at least 95%, such as at least 97%, such as at least 99%, such as 100% sequence identity to SEQ ID NO: 14.
22. The MSCs or the MSCs for use in a method of treatment of arthritis according to any one of the preceding items, wherein the mRNA construct comprises mRNA encoding an IL-10 of SEQ ID NO: 5 or a functional homologue of said IL-10 sharing at least 70%, such as at least 80%, for example at least 90%, such as at least 95% sequence identity therewith.
23. The MSCs or the MSCs for use in a method of treatment of arthritis according to any one of the preceding items, wherein the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 1.
24. The MSCs or the MSCs for use in a method of treatment of arthritis according to any one of the preceding items, wherein the mRNA construct comprises mRNA encoding IL-10 of SEQ ID NO: 5 or a functional homologue of said IL-10 sharing at least 70%, such as at least 80%, for example at least 90%, such as at least 95% sequence identity therewith and a signal peptide.
25. The MSCs or the MSCs for use in a method of treatment of arthritis according to any one of the preceding items, wherein the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 1 and a signal peptide.
26. The MSCs or the MSCs for use in a method of treatment of arthritis according to any one of the preceding items, wherein the mRNA construct encodes an IL-10 of SEQ ID NO: 6 or a functional homologue of said IL-10 sharing at least 70%, such as at least 80%, for example at least 90%, such as at least 95% sequence identity therewith.
27. The MSCs or the MSCs for use in a method of treatment of arthritis according to any one of the preceding items, wherein the mRNA construct comprises an mRNA construct having at least 70%, such as at least 80%, such as at least 85%, such as at least 90%, such as at least 95%, such as at least 97%, such as at least 99%, such as 100% sequence identity to SEQ ID NO: 2.
28. The MSCs or the MSCs for use in a method of treatment of arthritis according to any one of the preceding items, wherein the mRNA construct comprises mRNA encoding an IL1RN of SEQ ID NO: 11 or a functional homologue of said IL1RN sharing at least 70%, such as at least 80%, for example at least 90%, such as at least 95% sequence identity therewith.
29. The MSCs or the MSCs for use in a method of treatment of arthritis according to any one of the preceding items, wherein the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 7.
30. The MSCs or the MSCs for use in a method of treatment of arthritis according to any one of the preceding items, wherein the mRNA construct comprises mRNA encoding IL1RN of SEQ ID NO: 11 or a functional homologue of said IL1RN sharing at least 70%, such as at least 80%, for example at least 90%, such as at least 95% sequence identity therewith and a signal peptide.
31. The MSCs or the MSCs for use in a method of treatment of arthritis according to any one of the preceding items, wherein the mRNA construct comprises mRNA having at least 70%, such as at least 80%, such as at least 85%, such as at least 90%, such as at least 95%, such as at least 97%, such as at least 99%, such as 100% sequence identity to SEQ ID NO: 7 and a signal peptide.
32. The MSCs or the MSCs for use in a method of treatment of arthritis according to any one of the preceding items, wherein the mRNA construct encodes an IL1RN of SEQ ID NO: 12 or a functional homologue of said IL1RN sharing at least 70%, such as at least 80%, for example at least 90%, such as at least 95% sequence identity therewith.
33. The MSCs or the MSCs for use in a method of treatment of arthritis according to any one of the preceding items, wherein the mRNA construct comprises an mRNA construct having at least 70%, such as at least 80%, such as at least 85%, such as at least 90%, such as at least 95%, such as at least 97%, such as at least 99%, such as 100% sequence identity to SEQ ID NO: 8.
34. The MSCs or the MSCs for use in a method of treatment of arthritis according to any one of the preceding items, wherein the MSCs are selected from the group consisting of: Bone marrow derived MSCs, Adipose tissue derived MSCs, Umbilical cord tissue derived MSCs, Dental pulps derived MSCs, Endometrium derived MSCs, peripheral blood derived MSCs, skin derived MSCs, Placenta derived MSCs, Synovial fluid derived MSCs, muscle derived MSCs, Wharton's jelly derived MSCs and induced MSCs.
35. The MSCs or the MSCs for use in a method of treatment of arthritis according to any one of the preceding items, wherein the MSCs are induced mesenchymal stem cells (iMSCs).
36. The MSCs or the MSCs for use in a method of treatment of arthritis according to any one of items 1 to 34, wherein the MSCs are autologous MSCs from a mammal.
37. The MSCs or the MSCs for use in a method of treatment of arthritis according to item 36, wherein the MSCs are obtained from a mammalian donor, such as a dog, cat, horse or human.
38. The MSCs or the MSCs for use in a method of treatment of arthritis according to any one of the preceding items, wherein the MSCs possess the ability of self-replication.
39. The MSCs or the MSCs for use in a method of treatment of arthritis according to any one of the preceding items, wherein the MSCs are able to differentiate into osteoblasts, chondrocytes, myoblasts, adipocytes, stroma cells and/or tendon cells.
40. The MSCs or the MSCs for use in a method of treatment of arthritis according to any one of the preceding items, wherein the MSCs retain a surface marker profile characteristic of undifferentiated mesenchymal stem cells, including, for example, expression of CD73, CD90, and CD105, and absence of hematopoietic or lineage-specific markers such as CD34, CD45, CD19, CD11b, or HLA-DR.
41. The MSCs or the MSCs for use in a method of treatment of arthritis according to any one of the preceding items, wherein the MSCs exhibit reduced or undetectable expression levels of chondrogenic differentiation markers SOX9 and/or aggrecan (ACAN), relative to MSCs cultured under chondrogenic differentiation conditions.
42. The MSCs or the MSCs for use in a method of treatment of arthritis according to any one of the preceding items, wherein the MSCs exhibit reduced or undetectable expression levels of osteogenic differentiation markers RUNX2 and/or osteocalcin (BGLAP) relative to MSCs cultured under osteogenic differentiation conditions.
43. The MSCs or the MSCs for use in a method of treatment of arthritis according to any one of items 40 to 42, wherein the expression levels are determined by flow cytometry, RT-qPCR, or immunohistochemical analysis.
44. The MSCs or the MSCs for use in a method of treatment of arthritis according to any one the items 40 to 42, wherein the expression level of one or more markers selected from SOX9, collagen type II, RUNX2, and osteocalcin (BGLAP), as determined by immunohistochemistry, is reduced by at least 30%, 40%, or 50% relative to mesenchymal stem cells differentiated under chondrogenic or osteogenic induction conditions.
45. The MSCs or the MSCs for use in a method of treatment of arthritis according to any one of the preceding items, wherein the level of sulfated glycosaminoglycans (sGAGs) produced by the MSCs is reduced by at least 30%, 40%, or 50% relative to the level produced by mesenchymal stem cells cultured under chondrogenic induction conditions, as measured optionally by a dimethylmethylene blue (DMMB) assay.
46. The MSCs for use in a method of treatment of arthritis according to any one of the preceding items, wherein the cells are transfected with at least one mRNA construct, such as at least two different mRNA constructs, such as at least three different mRNA constructs, such as at least 4 different mRNA constructs, such as at least 5 different mRNA constructs, such as 6 different mRNA constructs.
47. The MSCs for use in a method of treatment of arthritis according to item 46, wherein the cells are transfected with an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 25 and one or more mRNA construct(s), such as one or more mRNA construct(s) comprising mRNA having at least 70%, such as at least 80%, such as at least 85%, such as at least 90%, such as at least 95%, such as at least 97%, such as at least 99%, such as 100% sequence identity to
   a. SEQ ID NO: 31; or
   b. SEQ ID NO: 1; or
   c. SEQ ID NO: 19; or
   d. SEQ ID NO: 13; or
   e. SEQ ID NO: 7.
48. The MSCs for use in a method of treatment of arthritis according to item 46, wherein the cells are transfected with an mRNA construct comprising mRNA encoding TGFβ3 of SEQ ID NO: 29 or a functional homologue of said TGFβ3 sharing at least 70%, such as at least 80%, for example at least 90%, such as at least 95% sequence identity therewith and one or more mRNA construct(s), such as one or more mRNA construct(s) comprising an mRNA construct encoding for a factor of the group comprising:
   a. WNT3a of SEQ ID NO: 35 or a functional homologue of said WNT3a sharing at least 70%, such as at least 80%, for example at least 90%, such as at least 95% sequence identity therewith; or
   b. IL-10 of SEQ ID NO: 5 or a functional homologue of said IL-10 sharing at least 70%, such as at least 80%, for example at least 90%, such as at least 95% sequence identity therewith; or
   c. IGF1 of SEQ ID NO: 23 or a functional homologue of said IGF1 sharing at least 70%, such as at least 80%, for example at least 90%, such as at least 95% sequence identity therewith;
   d. BMP6 of SEQ ID NO: 17 or a functional homologue of said BMP6 sharing at least 70%, such as at least 80%, for example at least 90%, such as at least 95% sequence identity therewith;
   e. IL1RN of SEQ ID NO: 11 or a functional homologue of said IL1RN sharing at least 70%, such as at least 80%, for example at least 90%, such as at least 95% sequence identity therewith.
49. The MSCs for use in a method of treatment of arthritis according to any one of items 46 to 48, wherein the ratio between the mRNA construct encoding for TGFβ3 and one of the mRNA constructs encoding one of the factors a to e is at least 1:0.5, such as at least 1:0.7, such as at least 1:0.9, such as at least 1:1, such as at least 1:1.1, such as at least 1:1.2, such as at least 1:1.5, such as at least 1:2.
50. The MSCs for use in a method of treatment of arthritis according to any one of items 46 to 49, wherein the mRNA construct(s) comprise a heterologous or native signal peptide.
51. The MSCs for use in a method of treatment of arthritis according to any one of the preceding items, wherein the arthritis is hemarthrosis, osteoarthritis, rheumatoid arthritis, Gout, septic arthritis, ankylosing spondylitis, Juvenile idiopathic arthritis, still's disease or psoriatic arthritis, preferably osteoarthritis or rheumatoid arthritis.
52. The MSCs for use in a method of treatment of arthritis according to any one of the preceding claims, wherein the MSCs are administered repeatedly to a subject.
53. The MSCs for use in a method of treatment of arthritis according to item 52, wherein the MSCs are administered at least once, such as at least twice, such as at least three times, such as at least 5 times.
54. The MSCs for use in a method of treatment of arthritis according to item 52, wherein the MSCs are administered at most 5 times, such as at most three times, such as at most twice, such as at most once.
55. The MSCs for use in a method of treatment of arthritis according to any one of items 52 to 54, wherein the MSCs are administered repeatedly to a subject, such as at least once a week, such as at least every two weeks, such as at least once a month, such as at least every two month, such as at least every six month, such as at least once a year, such as at least every second year, such as at least every 3 years.
56. The MSCs or the MSCs for use in a method of treatment of arthritis according to any one of the preceding items, wherein at least 1 ng mRNA construct, such as at least 10 ng mRNA construct, such as at least 100 ng mRNA construct, such as at least 0.5 g mRNA construct, such as at least 1 μg mRNA construct, such as at least 5 μg mRNA construct, such as at least 7 μg mRNA construct, such as at least 10 μg mRNA construct, such as at least 100 μg mRNA construct is introduced into 1×10⁶ mesenchymal stem cells.
57. The MSCs or the MSCs for use in a method of treatment of arthritis according to any one of the items 1 to 55, wherein at most 1 mg mRNA construct, such as at most 100 μg mRNA construct, such as 50 μg mRNA construct, such as at most 25 μg mRNA construct, such as at most 20 μg mRNA construct, such as at most 10 μg mRNA construct is introduced into 1×10⁶ mesenchymal stem cells.
58. The MSCs or the MSCs for use in a method of treatment of arthritis according to any one of the items 1 to 55, wherein between 1 ng mRNA construct and 1 mg mRNA construct, such as between 10 ng mRNA construct and 100 μg mRNA construct, such as between 0.5 μg mRNA construct and 50 μg mRNA construct, such as between 1 μg mRNA construct and 25 μg mRNA construct, such as between 5 μg mRNA construct and 20 μg mRNA construct is introduced into 1×10⁶ mesenchymal stem cells.
59. The MSCs or the MSCs for use in a method of treatment of arthritis according to any one of the items 1 to 55, wherein 1×10⁶ MSCs comprise at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct, such as at least 7 μg mRNA construct, such as at least 10 μg mRNA construct, such as at least 100 μg mRNA construct.
60. The MSCs or the MSCs for use in a method of treatment of arthritis according to any one of the items 1 to 55, wherein 1×10⁶ mesenchymal stem cells comprise at most 1 mg mRNA construct, such as at most 100 μg mRNA construct, such as 50 μg mRNA construct, such as at most 25 μg mRNA construct, such as at most 20 μg mRNA construct, such as at most 10 μg mRNA construct.
61. The MSCs or the MSCs for use in a method of treatment of arthritis according to any one of the items 1 to 55, wherein 1×10⁶ mesenchymal stem cells comprise between 1 ng mRNA construct and 1 mg mRNA construct, such as between 10 ng mRNA construct and 100 μg mRNA construct, such as between 0.5 μg mRNA construct and 50 μg mRNA construct, such as between 1 μg mRNA construct and 25 μg mRNA construct, such as between 5 μg mRNA construct and 20 μg mRNA construct.
62. The MSCs for use in a method of treatment of arthritis according to any one of the items 1 to 55, wherein 1×10⁶ MSCs comprise at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct, such as at least 7 μg mRNA construct, such as at least 10 μg mRNA construct, such as at least 100 μg mRNA construct at the time of administration.
63. The MSCs for use in a method of treatment of arthritis according to any one of the items 1 to 55, wherein 1×10⁶ mesenchymal stem cells comprise at most 1 mg mRNA construct, such as at most 100 μg mRNA construct, such as 50 μg mRNA construct, such as at most 25 μg mRNA construct, such as at most 20 μg mRNA construct, such as at most 10 μg mRNA construct at the time of administration.
64. The MSCs for use in a method of treatment of arthritis according to any one of the items 1 to 55, wherein 1×10⁶ mesenchymal stem cells comprise between 1 ng mRNA construct and 1 mg mRNA construct, such as between 10 ng mRNA construct and 100 g mRNA construct, such as between 0.5 μg mRNA construct and 50 μg mRNA construct, such as between 1 μg mRNA construct and 25 μg mRNA construct, such as between 5 μg mRNA construct and 20 μg mRNA construct. at the time of administration.
65. The MSCs for use in a method of treatment of arthritis according to any one of the items 1 to 55, wherein the MSCs are administered at most 1 minute, such as at most 10 minutes, such as at most 30 minutes, such as at most 1 hour, such as at most 2 hours, such as at most 3 hours, such as at most 4 hours, such as at most 5 hours, such as at most 10 hours, such as at most 12 hours, such as at most 15 hours, such as at most 24 hours, such as at most 36 hours, such as at most 48 hours after transfection.
66. The MSCs or the MSCs for use in a method of treatment of arthritis according to any one of the items 1 to 55, wherein the MSCs are cryopreserved at most 1 minute, such as at most 10 minutes, such as at most 30 minutes, such as at most 1 hour, such as at most 2 hours, such as at most 3 hours, such as at most 4 hours, such as at most 5 hours, such as at most 10 hours, such as at most 12 hours, such as at most 15 hours, such as at most 24 hours, such as at most 36 hours, such as at most 48 hours after transfection.
67. The MSCs or the MSCs for use in a method of treatment of arthritis according to any one of the items 1 to 55, wherein the transfected MSCs are administered at most 1 minute, such as at most 10 minutes, such as at most 30 minutes, such as at most 1 hour, such as at most 2 hours, such as at most 3 hours, such as at most 4 hours, such as at most 5 hours, such as at most 10 hours, such as at most 12 hours, such as at most 15 hours, such as at most 24 hours, such as at most 36 hours, such as at most 48 hours after thawing.
68. The MSCs or the MSCs for use in a method of treatment of arthritis according to any one of the items 56 to 68, wherein the method of detecting the amount of mRNA molecules in the mesenchymal stem cells is selected from the group consisting of: RNA sequencing, northern analysis, nuclease protection assays, In-situ hybridization, labelled mRNAs, ddPCR and RT-PCR.
69. The MSCs for use in a method of treatment of arthritis according to any one of items 52 to 68, wherein the subject is a subject diagnosed with arthritis.
70. The MSCs for use in a method of treatment of arthritis according to any one of items 52 to 69, wherein the MSCs are administered in combination with a treatment selected from the group consisting of acetaminophen, NSAID, glucosamine, chondroitin, opioid, steroid and lubricants, such as hyaluronic acid.
71. The MSCs for use in a method of treatment of arthritis according to any one of items 52 to 70, wherein the administration of the MSCs leads to joint damage repair.
72. The MSCs for use in a method of treatment of arthritis according to any one of items 52 to 70, wherein the administration of the MSCs leads to restoration of joint functions.
73. The MSCs for use in a method of treatment of arthritis according to any one of items 52 to 70, wherein the administration of the MSCs leads to pain relief.
74. The MSCs for use in a method of treatment of arthritis according to any one of items 52 to 73, wherein prior to administering the MSCs comprising an mRNA construct comprising mRNA encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding WNT3a of SEQ ID NO: 35 or a functional homologue of said WNT3a sharing at least 70%, such as at least 80%, for example at least 90%, such as at least 95% sequence identity therewith.
75. The MSCs for use in a method of treatment of arthritis according to any one of items 52 to 73, wherein prior to administering the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding IL-10 of SEQ ID NO: 5 or a functional homologue of said IL-10 sharing at least 70%, such as at least 80%, for example at least 90%, such as at least 95% sequence identity therewith.
76. The MSCs for use in a method of treatment of arthritis according to any one of items 52 to 73, wherein prior to administering the cells comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding IGF1 of SEQ ID NO: 23 or a functional homologue of said IGF1 sharing at least 70%, such as at least 80%, for example at least 90%, such as at least 95% sequence identity therewith.
77. The MSCs for use in a method of treatment of arthritis according to any one of items 52 to 73, wherein prior to administering the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding BMP6 of SEQ ID NO: 17 or a functional homologue of said BMP6 sharing at least 70%, such as at least 80%, for example at least 90%, such as at least 95% sequence identity therewith.
78. The MSCs for use in a method of treatment of arthritis according to any one of items 52 to 73, wherein prior to administering the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding IL1RN of SEQ ID NO: 11 or a functional homologue of said IL1RN sharing at least 70%, such as at least 80%, for example at least 90%, such as at least 95% sequence identity therewith.
79. The MSCs for use in a method of treatment of arthritis according to any one of items 74 to 78, wherein the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith are administered at least 1 day, such as at least 2 days, such as at least 5 days, such as at least 1 week, such as at least 2 weeks, such as at least 3 weeks, such as at least 4 weeks after administration of MSCs comprising mRNA construct encoding
   a. WNT3a of SEQ ID NO: 35 or a functional homologue of said WNT3a sharing at least 70%, such as at least 80%, for example at least 90%, such as at least 95% sequence identity therewith; or
   b. IL-10 of SEQ ID NO: 5 or a functional homologue of said IL-10 sharing at least 70%, such as at least 80%, for example at least 90%, such as at least 95% sequence identity therewith; or
   c. IGF1 of SEQ ID NO:23 or a functional homologue of said IGF1 sharing at least 70%, such as at least 80%, for example at least 90%, such as at least 95% sequence identity therewith;
   d. BMP6 of SEQ ID NO: 17 or a functional homologue of said BMP6 sharing at least 70%, such as at least 80%, for example at least 90%, such as at least 95% sequence identity therewith; or
   e. IL1RN of SEQ ID NO: 11 or a functional homologue of said IL1RN sharing at least 70%, such as at least 80%, for example at least 90%, such as at least 95% sequence identity therewith.
80. The MSCs for use in a method of treatment of arthritis according to any one of items 74 to 78, wherein the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith are administered at most 1 day, such as at most 2 days, such as at most 5 days, such as at most 1 week, such as at most 2 weeks, such as at most 3 weeks, such as at most 4 weeks after administration of MSCs comprising mRNA construct encoding
   a. WNT3a of SEQ ID NO: 35 or a functional homologue of said WNT3a sharing at least 70%, such as at least 80%, for example at least 90%, such as at least 95% sequence identity therewith; or
   b. IL-10 of SEQ ID NO: 5 or a functional homologue of said IL-10 sharing at least 70%, such as at least 80%, for example at least 90%, such as at least 95% sequence identity therewith; or
   c. IGF1 of SEQ ID NO: 23 or a functional homologue of said IGF1 sharing at least 70%, such as at least 80%, for example at least 90%, such as at least 95% sequence identity therewith;
   d. BMP6 of SEQ ID NO: 17 or a functional homologue of said BMP6 sharing at least 70%, such as at least 80%, for example at least 90%, such as at least 95% sequence identity therewith; or
   e. IL1RN of SEQ ID NO: 11 or a functional homologue of said IL1RN sharing at least 70%, such as at least 80%, for example at least 90%, such as at least 95% sequence identity therewith.
81. The MSCs for use in a method of treatment of arthritis according to any one of items 52 to 73, wherein after administering the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding WNT3a of SEQ ID NO: 35 or a functional homologue of said WNT3a sharing at least 70%, such as at least 80%, for example at least 90%, such as at least 95% sequence identity therewith.
82. The MSCs for use in a method of treatment of arthritis according to any one of items 52 to 73, wherein after administering the cells comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding IL-10 of SEQ ID NO: 5 or a functional homologue of said IL-10 sharing at least 70%, such as at least 80%, for example at least 90%, such as at least 95% sequence identity therewith.
83. The MSCs for use in a method of treatment of arthritis according to any one of items 52 to 73, wherein after administering the cells comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding IGF1 of SEQ ID NO: 23 or a functional homologue of said IGF1 sharing at least 70%, such as at least 80%, for example at least 90%, such as at least 95% sequence identity therewith.
84. The MSCs for use in a method of treatment of arthritis according to any one of items 52 to 73, wherein after administering the cells comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding BMP6 of SEQ ID NO: 17 or a functional homologue of said BMP6 sharing at least 70%, such as at least 80%, for example at least 90%, such as at least 95% sequence identity therewith.
85. The MSCs for use in a method of treatment of arthritis according to any one of items 52 to 73, wherein after administering the cells comprising an mRNA construct encoding TGFβ3 of SEQ ID NO: 29 or a functional homologue of said TGFβ3 sharing at least 70%, such as at least 80%, for example at least 90%, such as at least 95% sequence identity therewith, MSCs are administered comprising an mRNA construct encoding IL1RN of SEQ ID NO: 11 or a functional homologue of said IL1RN sharing at least 70%, such as at least 80%, for example at least 90%, such as at least 95% sequence identity therewith.
86. The MSCs for use in a method of treatment of arthritis according to any one of items 81 to 85, wherein the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith are administered at least 1 day, such as at least 2 days, such as at least 5 days, such as at least 1 week, such as at least 2 weeks, such as at least 3 weeks, such as at least 4 weeks before administration of MSCs comprising mRNA construct encoding
   a. WNT3a of SEQ ID NO: 35 or a functional homologue of said WNT3a sharing at least 70%, such as at least 80%, for example at least 90%, such as at least 95% sequence identity therewith; or
   b. IL-10 of SEQ ID NO: 5 or a functional homologue of said IL-10 sharing at least 70%, such as at least 80%, for example at least 90%, such as at least 95% sequence identity therewith; or
   c. IGF1 of SEQ ID NO: 23 or a functional homologue of said IGF1 sharing at least 70%, such as at least 80%, for example at least 90%, such as at least 95% sequence identity therewith;
   d. BMP6 of SEQ ID NO: 17 or a functional homologue of said BMP6 sharing at least 70%, such as at least 80%, for example at least 90%, such as at least 95% sequence identity therewith; or
   e. IL1RN of SEQ ID NO: 11 or a functional homologue of said IL1RN sharing at least 70%, such as at least 80%, for example at least 90%, such as at least 95% sequence identity therewith.
87. The MSCs for use in a method of treatment of arthritis according to any one of items 81 to 85, wherein the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith are administered at most 1 day, such as at most 2 days, such as at most 5 days, such as at most 1 week, such as at most 2 weeks, such as at most 3 weeks, such as at most 4 weeks before administration of MSCs comprising mRNA construct encoding
   a. WNT3a of SEQ ID NO: 35 or a functional homologue of said WNT3a sharing at least 70%, such as at least 80%, for example at least 90%, such as at least 95% sequence identity therewith; or
   b. IL-10 of SEQ ID NO: 5 or a functional homologue of said IL-10 sharing at least 70%, such as at least 80%, for example at least 90%, such as at least 95% sequence identity therewith; or
   c. IGF1 of SEQ ID NO: 23 or a functional homologue of said IGF1 sharing at least 70%, such as at least 80%, for example at least 90%, such as at least 95% sequence identity therewith;
   d. BMP6 of SEQ ID NO: 17 or a functional homologue of said BMP6 sharing at least 70%, such as at least 80%, for example at least 90%, such as at least 95% sequence identity therewith; or
   e. IL1RN of SEQ ID NO: 11 or a functional homologue of said IL1RN sharing at least 70%, such as at least 80%, for example at least 90%, such as at least 95% sequence identity therewith.
88. The MSCs for use in a method of treatment of arthritis according to any one of items 52 to 73, wherein the MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith are administered simultaneously with MSCs comprising mRNA construct encoding
   a. WNT3a of SEQ ID NO: 35 or a functional homologue of said WNT3a sharing at least 70%, such as at least 80%, for example at least 90%, such as at least 95% sequence identity therewith; or
   b. IL-10 of SEQ ID NO: 5 or a functional homologue of said IL-10 sharing at least 70%, such as at least 80%, for example at least 90%, such as at least 95% sequence identity therewith; or
   c. IGF1 of SEQ ID NO: 23 or a functional homologue of said IGF1 sharing at least 70%, such as at least 80%, for example at least 90%, such as at least 95% sequence identity therewith;
   d. BMP6 of SEQ ID NO: 17 or a functional homologue of said BMP6 sharing at least 70%, such as at least 80%, for example at least 90%, such as at least 95% sequence identity therewith; or
   e. IL1RN of SEQ ID NO: 11 or a functional homologue of said IL1RN sharing at least 70%, such as at least 80%, for example at least 90%, such as at least 95% sequence identity therewith.
89. The MSCs for use in a method of treatment of arthritis according to any one of the preceding items, wherein at least 1×10⁵, such as at least 5×10⁵, such as at least 1×10⁶, such as at least 5×10⁶, such as at least 1×10⁷, such as at least 5×10⁷ cells are administered to the subject.
90. The MSCs for use in a method of treatment of arthritis according to any one of items 48, 74, 79, 80, 81, 86, 87 and 88, wherein WNT3a is encoded by an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 31 or SEQ ID NO: 32.
91. The MSCs for use in a method of treatment of arthritis according to any one of items 48, 75, 79, 80, 82, 86, 87 and 88, wherein IL-10 is encoded by an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 1 or SEQ ID NO: 2.
92. The MSCs for use in a method of treatment of arthritis according to any one of items 48, 76, 79, 80, 83, 86, 87 and 88, wherein IGF1 is encoded by an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 19 or SEQ ID NO: 20.
93. The MSCs for use in a method of treatment of arthritis according to any one of items 48, 77, 79, 80, 84, 86, 87 and 88, wherein BMP6 is encoded by an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 13 or SEQ ID NO: 14.
94. The MSCs for use in a method of treatment of arthritis according to any one of items 48, 78, 79, 80, 85, 86, 87 and 88, wherein IL1RN is encoded by an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 7 or SEQ ID NO: 8.
95. The MSCs for use in a method of treatment of arthritis according to any one of the preceding claims, wherein the cells are cryopreserved and/or frozen and subsequently thawed prior to administering to the subject.
96. The MSCs for use in a method of treatment of arthritis according to item 95, wherein the cells are cryopreserved in 5% to 10% DMSO.
97. A kit of parts of use in a method of treatment of arthritis and/or pain, said kit of part comprising:
   a. MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith; and
   b. MSCs comprising mRNA construct encoding
      i. IL-10 of SEQ ID NO: 5 IL-10 of SEQ ID NO: 5 or a functional homologue of said IL-10 sharing at least 70%, such as at least 80%, for example at least 90%, such as at least 95% sequence identity therewith; or
      ii. WNT3a of SEQ ID NO: 35 or a functional homologue of said WNT3a sharing at least 70%, such as at least 80%, for example at least 90%, such as at least 95% sequence identity therewith; or
      iii. IGF1 of SEQ ID NO:23 or a functional homologue of said IGF1 sharing at least 70%, such as at least 80%, for example at least 90%, such as at least 95% sequence identity therewith;
      iv. BMP6 of SEQ ID NO: 17 or a functional homologue of said BMP6 sharing at least 70%, such as at least 80%, for example at least 90%, such as at least 95% sequence identity therewith; or
      v. IL1RN of SEQ ID NO: 11 or a functional homologue of said IL1RN sharing at least 70%, such as at least 80%, for example at least 90%, such as at least 95% sequence identity therewith;
         wherein said method comprises administering the MSC of a) at least 1 day, such as at least 2 days, such as at least 5 days, such as at least 1 week, such as at least 2 weeks, such as at least 3 weeks, such as at least 4 weeks after the MSCs of b).
98. A kit of parts of use in a method of treatment of arthritis and/or pain, said kit of part comprising:
   a. MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith; and
   b. MSCs comprising mRNA construct encoding
      i. IL-10 of SEQ ID NO: 5 IL-10 of SEQ ID NO: 5 or a functional homologue of said IL-10 sharing at least 70%, such as at least 80%, for example at least 90%, such as at least 95% sequence identity therewith; or
      ii. WNT3a of SEQ ID NO: 35 or a functional homologue of said WNT3a sharing at least 70%, such as at least 80%, for example at least 90%, such as at least 95% sequence identity therewith; or
      iii. IGF1 of SEQ ID NO:23 or a functional homologue of said IGF1 sharing at least 70%, such as at least 80%, for example at least 90%, such as at least 95% sequence identity therewith;
      iv. BMP6 of SEQ ID NO: 17 or a functional homologue of said BMP6 sharing at least 70%, such as at least 80%, for example at least 90%, such as at least 95% sequence identity therewith; or
      v. IL1RN of SEQ ID NO: 11 or a functional homologue of said IL1RN sharing at least 70%, such as at least 80%, for example at least 90%, such as at least 95% sequence identity therewith;
         wherein said method comprises administering the MSC of a) at most 1 day, such as at most 2 days, such as at most 5 days, such as at most 1 week, such as at most 2 weeks, such as at most 3 weeks, such as at most 4 weeks after the MSCs of b).
99. A kit of parts of use in a method of treatment of arthritis and/or pain, said kit of part comprising:
   a. MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith; and
   b. MSCs comprising mRNA construct encoding
      i. IL-10 of SEQ ID NO: 5 IL-10 of SEQ ID NO: 5 or a functional homologue of said IL-10 sharing at least 70%, such as at least 80%, for example at least 90%, such as at least 95% sequence identity therewith; or
      ii. WNT3a of SEQ ID NO: 35 or a functional homologue of said WNT3a sharing at least 70%, such as at least 80%, for example at least 90%, such as at least 95% sequence identity therewith; or
      iii. IGF1 of SEQ ID NO:23 or a functional homologue of said IGF1 sharing at least 70%, such as at least 80%, for example at least 90%, such as at least 95% sequence identity therewith;
      iv. BMP6 of SEQ ID NO: 17 or a functional homologue of said BMP6 sharing at least 70%, such as at least 80%, for example at least 90%, such as at least 95% sequence identity therewith; or
      v. IL1RN of SEQ ID NO: 11 or a functional homologue of said IL1RN sharing at least 70%, such as at least 80%, for example at least 90%, such as at least 95% sequence identity therewith;
         wherein said method comprises administering the MSC of a) at least 1 day, such as at least 2 days, such as at least 5 days, such as at least 1 week, such as at least 2 weeks, such as at least 3 weeks, such as at least 4 weeks before the MSCs of b).
100. A kit of parts of use in a method of treatment of arthritis and/or pain, said kit of part comprising:
   a. MSCs comprising an mRNA construct encoding TGFβ3 of SEQ ID NO: 29 or a functional homologue of said TGFβ3 sharing at least 70%, such as at least 80%, for example at least 90%, such as at least 95% sequence identity therewith; and
   b. MSCs comprising mRNA construct encoding
      i. IL-10 of SEQ ID NO: 5 IL-10 of SEQ ID NO: 5 or a functional homologue of said IL-10 sharing at least 70%, such as at least 80%, for example at least 90%, such as at least 95% sequence identity therewith; or
      ii. WNT3a of SEQ ID NO: 35 or a functional homologue of said WNT3a sharing at least 70%, such as at least 80%, for example at least 90%, such as at least 95% sequence identity therewith; or
      iii. IGF1 of SEQ ID NO:23 or a functional homologue of said IGF1 sharing at least 70%, such as at least 80%, for example at least 90%, such as at least 95% sequence identity therewith;
      iv. BMP6 of SEQ ID NO: 17 or a functional homologue of said BMP6 sharing at least 70%, such as at least 80%, for example at least 90%, such as at least 95% sequence identity therewith; or
      v. IL1RN of SEQ ID NO: 11 or a functional homologue of said IL1RN sharing at least 70%, such as at least 80%, for example at least 90%, such as at least 95% sequence identity therewith;
         wherein said method comprises administering the MSC of a) at most 1 day, such as at most 2 days, such as at most 5 days, such as at most 1 week, such as at most 2 weeks, such as at most 3 weeks, such as at most 4 weeks before the MSCs of b).
101. A kit of parts of use in a method of treatment of arthritis and/or pain, said kit of part comprising:
   a. MSCs comprising an mRNA construct encoding WNT3a of SEQ ID NO: 35 or a functional homologue of said WNT3a sharing at least 70%, such as at least 80%, for example at least 90%, such as at least 95% sequence identity therewith; and
   b. MSCs comprising mRNA construct encoding
      i. IL-10 of SEQ ID NO: 5 IL-10 of SEQ ID NO: 5 or a functional homologue of said IL-10 sharing at least 70%, such as at least 80%, for example at least 90%, such as at least 95% sequence identity therewith; or
      ii. WNT3a of SEQ ID NO: 35 or a functional homologue of said WNT3a sharing at least 70%, such as at least 80%, for example at least 90%, such as at least 95% sequence identity therewith; or
      iii. IGF1 of SEQ ID NO:23 or a functional homologue of said IGF1 sharing at least 70%, such as at least 80%, for example at least 90%, such as at least 95% sequence identity therewith;
      iv. BMP6 of SEQ ID NO: 17 or a functional homologue of said BMP6 sharing at least 70%, such as at least 80%, for example at least 90%, such as at least 95% sequence identity therewith; or
      v. IL1RN of SEQ ID NO: 11 or a functional homologue of said IL1RN sharing at least 70%, such as at least 80%, for example at least 90%, such as at least 95% sequence identity therewith;
         wherein said method comprises administering the MSC of a) simultaneously with the MSCs of b).
102. The MSCs according to any one of items 1 to 36 for use in a method of treatment of pain.
103. The MSCs according to 102, wherein the administration of the MSCs leads to nociceptive pain relief, such as complete removal of the nociceptive pain or a reduction in the severity of the nociceptive pain.
104. The MSCs for use in a method of treatment of arthritis according to item 102, wherein the administration of the MSCs leads to inflammatory pain relief, such as complete removal of the inflammatory pain or a reduction in the severity of the inflammatory pain.
105. The MSCs according to item 102, wherein administration of the MSCs leads to degenerative pain relief, such as complete removal of the degenerative pain or a reduction in the severity of the degenerative pain.
106. The MSCs according to item 102, wherein the administration of the MSCs leads to neuropathic pain relief, such as complete removal of the neuropathic pain or a reduction in the severity of the neuropathic pain.
107. The MSCs according to any one of the items 103 to 106, wherein the pain relief is measured using one or more standardised clinical tools, such as the Visual Analogue Scale (VAS), Numerical Rating Scale (NRS), Brief Pain Inventory (BPI), or Western Ontario and McMaster Universities Osteoarthritis Index (WOMAC).
108. The MSCs according to any one of the items 103 to 107, wherein administration results in a reduction of at least 30%, such as at least 50%, in the pain score assessed by a standardised clinical instrument.
109. A method of generating MSCs according to any one of items 1 to 96, wherein the method comprises:
   a) Providing MSCs; and
   b) Transfecting the MSCs with an mRNA construct encoding TGFβ3 of SEQ ID NO: 29 or a functional homologue of said TGFβ3 sharing at least 70%, such as at least 80%, for example at least 90%, such as at least 95% sequence identity therewith.
110. The method according to item 109, wherein the MSCs in step a) are cultured for at least 1, such as at least 2, such as at least 5, such as at least 10 passages before transfection.
111. The method according to item 109, wherein the MSCs in step a) are cultured for at most 10, such as at most 5, such as at most 2, such as at most 1 passage(s) before transfection.
112. The method according to any one of items 109 to 111, wherein the MSCs are generated by
   a. culturing human pluripotent stem cells in a culture media comprising a WNT pathway agonist and a BET pathway antagonist for at least two days to generate induced cells; and
   b. culturing the induced cells from step (a) in a culture media comprising a PDGF pathway agonist, an IGF1 pathway agonist and an FGF-beta pathway agonist for at least ten days.
113. The method according to item 112, wherein the human pluripotent stem cells are human embryonic stem cells.
114. The method according to any one of items 112 to 113, wherein the WNT pathway agonist is selected from the group consisting of CHIR98014, CHIR99021, SB 216763, SB 415286, LY2090314, 3F8, A 1070722, AR-A 014418, BIO, AZD1080, WNT3A, and combinations thereof.
115. The method according to any one of items 112 to 114, wherein the WNT pathway agonist is present in the culture media at a concentration within a range of 0.25-0.75 μM.
116. The method according to any one of items 112 to 115, wherein the BET pathway antagonist is selected from the group consisting of (+)-JQ1, TEN-010, OTX015, I-BET762, I-BET151, BAY1238097, ABBV-744, ABBV-075, iBET-BD1, iBET-BD2, SJ432, RVX-208, MS417, AZD5153, and combinations thereof.
117. The method according to any one of items 112 to 116, wherein the BET pathway antagonist is present in the culture media at a concentration within a range of 25-75 nM.
118. The method according to any one of items 112 to 117, wherein the PDGF pathway agonist is PDGF-BB.
119. The method according to item 118, wherein PDGF-BB is present in the culture media at a concentration within a range of 7.5-12.5 ng/ml.
120. The method according to any one of items 112 to 119, wherein the IGF1 pathway agonist is IGF1.
121. The method according to item 120, wherein IGF1 is present in the culture media at a concentration within a range of 15-25 ng/ml.
122. The method according to any one of items 112 to 121, wherein the FGF-beta pathway agonist is FGF-beta.
123. The method according to item 122, wherein FGF-beta is present in the culture media at a concentration within a range of 7.5-12.5 ng/ml.
124. The method according to any one of items 109 to 123, wherein the mRNA construct is introduced into the MSCs by a non-endosomal pathway of delivery.
125. The method according to item 124, wherein the non-endosomal pathway of delivery is selected from the group consisting of: biolistic particle delivery, ultrasonic nebulization, sonification, buffer-mediated delivery, delivery utilizing phagocytosis, pinocytosis, clathrin-dependent delivery and/or clathrin-independent delivery by virus particles and/or polymers, microinjection, streptolysin-O permeabilization, permeabilization using anionic peptides and electroporation.
126. The method according to any one of items 124 to 125, wherein the non-endosomal pathway of delivery is electroporation.
127. The method according to any one of the items 109 to 126, wherein at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct, such as at least 7 μg mRNA construct, such as at least 10 μg mRNA construct, such as at least 100 μg mRNA construct is introduced into 1×10⁶ mesenchymal stem cells.
128. The method according to any one of the items 109 to 126, wherein at most 1 mg mRNA construct, such as at most 100 μg mRNA construct, such as 50 μg mRNA construct, such as at most 25 μg mRNA construct, such as at most 20 μg mRNA construct, such as at most 10 μg mRNA construct is introduced into 1×10⁶ mesenchymal stem cells.
129. The method according to any one of items 109 to 126, wherein between 1 ng mRNA construct and 1 mg mRNA construct, such as between 10 ng mRNA construct and 100 μg mRNA construct, such as between 0.5 μg mRNA construct and 50 μg mRNA construct, such as between 1 μg mRNA construct and 25 μg mRNA construct, such as between 5 μg mRNA construct and 20 μg mRNA construct is introduced into 1×10⁶ mesenchymal stem cells.
130. The method according to any one of items 127 to 129, wherein, the method of detecting the amount of mRNA molecules in the mesenchymal stem cells is selected from the group consisting of: RNA sequencing, northern analysis, nuclease protection assays, In-situ hybridization and RT-PCR.
131. The method according to any one of items 109 to 130, wherein the mRNA construct comprises only unmodified nucleotide bases.
132. The method according to any one of items 109 to 131, wherein the mRNA construct is codon optimized
   a. to enhance the expression in a subject; or
   b. to optimize the uridine content; or
   c. to increase the stability in the MSCs.
133. The method according to any one of items 109 to 132, wherein the MSCs comprise a substantially single stranded composition of a mRNA construct introduced into the cell by a non-endosomal pathway of delivery, the mRNA construct comprising a coding sequence, wherein all nucleosides within the mRNA construct are chemically unmodified.
134. The method according to any one of items 131 to 133, wherein the mRNA construct has been designed to reduce the uridine content.
135. The method according to any one of items 133 to 134, wherein the method to remove dsRNA of the mRNA composition is selected from the group consisting of: cellulose chromatography, High-performance liquid chromatography, silica based chromatography and polyA affinity annealing.
136. The method according to any one of items 133 to 135, wherein the mRNA construct is purified at least once before transfection, such as at least twice, such as at least three times.
137. The method according to any one of items 133 to 136, wherein the method to detect the amount of dsRNA in the mRNA construct is selected from the group consisting of: Dot blot, lateral flow immunoassay, ELISA, electrophoresis, northernblot, spectroscopy e.g., mass spectroscopy and dsRNA-sequencing.
138. The method according to any one of items 132 to 137, wherein the codons of the coding sequence have been selected to reduce the uridine content by a method, wherein the method comprises following steps:
   a. Codon optimization for expression in humans;
   b. Reduction of uridine content by selecting uridine low or uridine free codons.
139. The method according to item 138, wherein the codons are optimized to rebalance codon usage, decrease sequence complexity, avoid rare codons, minimize secondary structures and/or reduce complexity.
140. The method according to any one of items 132 to 139, wherein the absolute uridine content of the mRNA construct in the mRNA composition is reduced by at least 0.5%, such as at least 1%, such as at least 2.5%, such as at least 5%, such as at least 10%, such as at least 15% compared to the uridine content of the naturally occurring mRNA.
141. The method according to any one of items 132 to 139, wherein the absolute uridine content of the mRNA construct in the mRNA composition is reduced by at most 15%, such as at most 10%, such as at most 5% compared to the uridine content of the naturally occurring mRNA.
142. The method according to any one of items 132 to 139, wherein the absolute uridine content of the mRNA construct in the mRNA composition is reduced by between 0.5% and 15%, such as between 0.5% and 15%, such as between 2.5% and 15%, such as between 5% and 15%, such as by between 10% and 15% compared to the uridine content of the naturally occurring mRNA.
143. The method according to any one of items 109 to 130, wherein the mRNA construct comprises at least one modified nucleotide base.
144. The method according to item 143, wherein at least one uridine is replaced with a modified nucleotide base, such as Pseudouridine or N1-methyl-pseudouridine.
145. The method according to any one of items 143 to 144, wherein at least one cytosine is replaced with a modified nucleotide base, such as 5-methylcytosine.
146. The method according to any one of items 109 to 145, wherein the mRNA construct comprises a 3′ and/or 5′ untranslated region (UTR).
147. The method according to any one of items 109 to 146, wherein the mRNA construct comprises a 5′ cap.
148. The method according to any one of items 109 to 147, wherein the mRNA construct comprises a poly-A tail.
149. The method according to any one of items 109 to 148, wherein the mRNA construct comprises a 5′ untranslated region (UTR), a 3′ untranslated region (UTR), a 5′ cap, and a poly-A tail.
150. The method according to any one of items 109 to 149, wherein the method to synthesize the mRNA construct is selected from the group of Enzymatic (IVT) methods, solid-phase methods, liquid-phase methods, combined synthetic methods, small region synthesis, and ligation methods.
151. The method according to item 150, wherein a T7 polymerase is used for synthesizing the mRNA construct.
152. The method according to any one of items 109 to 151, wherein the mRNA construct can be detected in the MSCs at least 1 day, such as at least 2 days, such as at least 3 days, such as at least 5 days, such as at least 7 days, such as at least 8 days, such as at least 10 days, such as at least 12 days, such as at least 15 days, such as at least 20 days after transfection.
153. The method according to any one of items 109 to 152, wherein after 1 day at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct per 1×10⁶ mesenchymal stem cells can be detected.
154. The method according to any one of items 109 to 153, wherein after 2 days at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct per 1×10⁶ mesenchymal stem cells can be detected.
155. The method according to any one of items 109 to 154, wherein after 3 days at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA per 1×10⁶ mesenchymal stem cells can be detected.
156. The method according to any one of items 109 to 155, wherein after 5 days at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct per 1×10⁶ mesenchymal stem cells can be detected.
157. The method according to any one of items 109 to 156, wherein after 7 days at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct per 1×10⁶ mesenchymal stem cells can be detected.
158. The method according to any one of items 109 to 157, wherein after 8 days at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct per 1×10⁶ mesenchymal stem cells can be detected.
159. The method according to any one of items 109 to 158, wherein after 10 days at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct per 1×10⁶ mesenchymal stem cells can be detected.
160. The method according to any one of items 109 to 159, wherein after 12 days at least 1 ng mRNA construct, such as at least 10 ng mRNA construct, such as at least 100 ng mRNA construct, such as at least 0.5 μg mRNA construct, such as at least 1 μg mRNA construct, such as at least 5 μg mRNA construct per 1×10⁶ mesenchymal stem cells can be detected.
161. The method according to any one of items 152 to 160, wherein, the method of detecting the amount of mRNA molecules in the mesenchymal stem cells is selected from the group consisting of: RNA sequencing, northern analysis, nuclease protection assays, In-situ hybridization and RT-PCR.
162. The method according to any one of items 109 to 161, wherein the protein encoded on said mRNA construct can be detected at least 1 day, such as at least 2 days, such as at least 3 days, such as at least 5 days, such as at least 7 days, such as at least 8 days, such as at least 10 days, such as at least 12 days, such as at least 15 days, such as at least 20 days after transfection in the supernatant.
163. The method according to any one of items 109 to 162, wherein 1 day after transfection 1000 cells express at least 10 pg, such as at least 100 pg, such as at least 1 ng, such as at least 1.25 ng, such as at least 1.5 ng, such as at least 1.75 ng, such as at least 2 ng protein.
164. The method according to any one of items 109 to 163, wherein 2 days after transfection 1000 cells express at least 10 pg, such as at least 100 pg, such as at least 1 ng, such as at least 1.25 ng, such as at least 1.5 ng, such as at least 1.75 ng, such as at least 2 ng protein.
165. The method according to any one of the items 109 to 164, wherein 3 days after transfection 1000 cells express at least 10 pg, such as at least 100 pg, such as at least 1 ng, such as at least 1.25 ng, such as at least 1.5 ng, such as at least 1.75 ng, such as at least 2 ng protein.
166. The method according to any one of the items 109 to 165, wherein 5 days after transfection 1000 cells express at least 10 pg, such as at least 100 pg, such as at least 1 ng, such as at least 1.25 ng, such as at least 1.5 ng, such as at least 1.75 ng, such as at least 2 ng protein.
167. The method according to any one of the items 109 to 166, wherein 7 days after transfection 1000 cells express at least 10 pg, such as at least 100 pg, such as at least 1 ng, such as at least 1.25 ng, such as at least 1.5 ng, such as at least 1.75 ng, such as at least 2 ng protein.
168. The method according to any one of the items 109 to 167, wherein 8 days after transfection 1000 cells express at least 10 pg, such as at least 100 pg, such as at least 1 ng, such as at least 1.25 ng, such as at least 1.5 ng, such as at least 1.75 ng, such as at least 2 ng protein.
169. The method according to any one of the items 109 to 168, wherein 10 days after transfection 1000 cells express at least 10 pg, such as at least 100 pg, such as at least 1 ng, such as at least 1.25 ng, such as at least 1.5 ng, such as at least 1.75 ng, such as at least 2 ng protein.
170. The method according to any one of the items 109 to 169, wherein 12 days after transfection 1000 cells express at least 10 pg, such as at least 100 pg, such as at least 1 ng, such as at least 1.25 ng, such as at least 1.5 ng, such as at least 1.75 ng, such as at least 2 ng protein.
171. A composition comprising MSCs according to any one of items 1 to 96.
172. A method of expressing TGFβ3, the method comprising introducing an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 25 into MSCs according to any one of the items 1 to 96 by a non-endosomal pathway of delivery.
173. An mRNA construct comprising mRNA having at least 70%, such as at least 80%, such as at least 85%, such as at least 90%, such as at least 95%, such as at least 97%, such as at least 99%, such as 100% sequence identity to SEQ ID NO: 25.
174. An engineered MSC comprising an mRNA construct comprising mRNA having at least 70%, such as at least 80%, such as at least 85%, such as at least 90%, such as at least 95%, such as at least 97%, such as at least 99%, such as 100% sequence identity to SEQ ID NO: 25.
175. The engineered MSC according to item 174, wherein said MSC is generated by a method according to any one of items 109 to 170.
176. The MSCs for use in a method of treatment of arthritis according to any one of item 1 to 96, wherein said MSCs are generated by a method according to items 109 to 170.
177. The MSCs for use in a method of treatment of arthritis according to any one of item 1 to 96, wherein said MSCs are MSCs according to any one of items 174 to 175.
178. The MSCs for use in a method of treatment of pain according to any one of item 102 to 106, wherein said MSCs are generated by a method according to items 109 to 170.
179. The MSCs for use in a method of treatment of pain according to any one of item 102 to 106, wherein said MSCs are MSCs according to any one of items 174 to 175.
180. A method according to the disclosure, wherein one MSC comprises at least 1×10⁴ copies of the mRNA construct, such as at least 5×10⁴ copies of the mRNA construct, such as at least 1×10⁵ copies of the mRNA construct, such as at least 2×10⁵ copies of the mRNA construct, such as at least 3×10⁵ copies of the mRNA construct at the time of administration.
181. A method of treatment of arthritis in a subject, the method comprising administering to the subject mesenchymal stem cells (MSCs), wherein the MSCs are transfected with an mRNA construct comprising mRNA encoding a TGFβ3 of SEQ ID NO: 29 or a functional homologue of said TGFβ3 sharing at least 70% sequence identity, such as at least 80%, such as at least 85%, such as at least 90%, such as at least 95%, such as at least 97%, such as at least 99%, such as 100% sequence identity, therewith; and wherein the mRNA construct further comprises mRNA encoding a WNT3a of SEQ ID NO: 35 or a functional homologue of said WNT3a sharing at least 70% sequence identity, such as at least 80%, such as at least 85%, such as at least 90%, such as at least 95%, such as at least 97%, such as at least 99%, such as 100% sequence identity, therewith.
182. The method according to item 181, wherein the mRNA construct encoding WNT3a comprises mRNA having at least 70% sequence identity to SEQ ID NO: 31.
183. A kit of parts of use in treatment of arthritis and/or pain, said kit of parts comprising:
   a. Mesenchymal stem cells (MSCs) comprising an mRNA construct encoding TGFβ3 of SEQ ID NO: 29 or a functional homologue of said TGFβ3 sharing at least 70% sequence identity therewith;
   b. MSCs comprising mRNA construct encoding
      i. IL-10 of SEQ ID NO: 5 or a functional homologue of said IL-10 sharing at least 70% sequence identity therewith; or
      ii. WNT3a of SEQ ID NO: 35 or a functional homologue of said WNT3a sharing at least 70% sequence identity therewith; or
      iii. IGF1 of SEQ ID NO:23 or a functional homologue of said IGF1 sharing at least 70% sequence identity therewith; or
      iv. BMP6 of SEQ ID NO: 17 or a functional homologue of said BMP6 sharing at least 70% sequence identity therewith; or
      v. IL1RN of SEQ ID NO: 11 or a functional homologue of said IL1RN sharing at least 70% sequence identity therewith; and
      c. Instructions for use in a subject comprising administering the MSC of a) at least 1 day after the MSCs of b).
184. A kit of parts of use in treatment of arthritis and/or pain, said kit of parts comprising:
   c. Mesenchymal stem cells (MSCs) comprising an mRNA construct encoding TGFβ3 of SEQ ID NO: 29 or a functional homologue of said TGFβ3 sharing at least 70% sequence identity therewith;
   d. MSCs comprising mRNA construct encoding
      vi. IL-10 of SEQ ID NO: 5 or a functional homologue of said IL-10 sharing at least 70% sequence identity therewith; or
      vii. WNT3a of SEQ ID NO: 35 or a functional homologue of said WNT3a sharing at least 70% sequence identity therewith; or
      viii. IGF1 of SEQ ID NO:23 or a functional homologue of said IGF1 sharing at least 70% sequence identity therewith; or
      ix. BMP6 of SEQ ID NO: 17 or a functional homologue of said BMP6 sharing at least 70% sequence identity therewith; or
      x. IL1RN of SEQ ID NO: 11 or a functional homologue of said IL1RN sharing at least 70% sequence identity therewith; and
   c. Instructions for use in a subject comprising administering the MSC of a) at least 1 day before the MSCs of b).

1. A method of treatment of arthritis in a subject, the method comprising administering to the subject mesenchymal stem cells (MSCs), wherein the MSCs are transfected with an mRNA construct comprising mRNA encoding a TGFβ3 of SEQ ID NO: 29 or a functional homologue of said TGFβ3 sharing at least 70% sequence identity therewith.

1. The method according to claim 1, wherein the mRNA construct comprises mRNA having at least 70% sequence identity to SEQ ID NO: 25.

2. The method according to claim 1, wherein the mRNA construct comprises mRNA encoding TGFβ3 of SEQ ID NO: 29 or a functional homologue of said TGFβ3 sharing at least 70% sequence identity therewith and a signal peptide.

3. The method according to claim 1, wherein the mRNA construct comprises mRNA having at least 70% sequence identity to SEQ ID NO: 25 and a signal peptide.

4. The method according to claim 3, wherein the signal peptide is a heterologous signal peptide.

5. The method according to claim 1, wherein the mRNA construct further comprises mRNA encoding an IGF1 of SEQ ID NO: 23 or a functional homologue of said IGF1 sharing at least 70% sequence identity therewith.

6. The method according to claim 6, wherein the mRNA construct comprises mRNA having at least 70% sequence identity to SEQ ID NO: 19.

7. The method according to claim 1, wherein the mRNA construct further comprises mRNA encoding a BMP6 of SEQ ID NO: 17 or a functional homologue of said BMP6 sharing at least 70% sequence identity therewith.

8. The method according to claim 8, wherein the mRNA construct comprises mRNA having at least 70% sequence identity to SEQ ID NO: 13.

9. The method according to claim 1, wherein the mRNA construct further comprises mRNA encoding an IL-10 of SEQ ID NO: 5 or a functional homologue of said IL-10 sharing at least 70% sequence identity therewith.

10. The method according to claim 10, wherein the mRNA construct comprises mRNA having at least 70% sequence identity to SEQ ID NO: 1.

11. The method according to claim 1, wherein the mRNA construct further comprises mRNA encoding an IL1RN of SEQ ID NO: 11 or a functional homologue of said IL1RN sharing at least 70% sequence identity therewith.

12. The method according to claim 12, wherein the mRNA construct comprises mRNA having at least 70% sequence identity to SEQ ID NO: 7.

13. The method according to claim 1, wherein the mRNA construct further comprises mRNA encoding a WNT3a of SEQ ID NO: 35 or a functional homologue of said WNT3a sharing at least 70% sequence identity therewith.

14. The method according to claim 14, wherein the mRNA construct comprises mRNA having at least 70% sequence identity to SEQ ID NO: 31.

15. The method according to claim 3, wherein the signal peptide is a native signal peptide.

16. The method according to claim 1, wherein the mRNA construct is a fully unmodified mRNA construct, wherein all nucleosides in the mRNA construct are chemically unmodified.

17. The method according to claim 1, wherein the MSCs are induced mesenchymal stem cells (iMSCs).

18. The method according to claim 1, wherein the MSCs are able to differentiate into osteoblasts, chondrocytes, myoblasts, adipocytes, stroma cells and/or tendon cells.

19. The method according to claim 1, wherein the MSCs exhibit reduced or undetectable expression levels of chondrogenic differentiation markers SOX9 and/or aggrecan (ACAN), relative to MSCs cultured under chondrogenic differentiation conditions.

20. The method according to claim 1, wherein the MSCs exhibit reduced or undetectable expression levels of osteogenic differentiation markers RUNX2 and/or osteocalcin (BGLAP) relative to MSCs cultured under osteogenic differentiation conditions.

21. The method according to claim 1, wherein the level of sulfated glycosaminoglycans (sGAGs) produced by the MSCs is reduced by at least 30%, 40%, or 50% relative to the level produced by mesenchymal stem cells cultured under chondrogenic induction conditions, as measured optionally by a dimethylmethylene blue (DMMB) assay.

22. The method according to claim 1, wherein the arthritis is hemarthrosis, osteoarthritis, rheumatoid arthritis, Gout, septic arthritis, ankylosing spondylitis, Juvenile idiopathic arthritis, still's disease or psoriatic arthritis.

23. The method according to claim 1, wherein 1×10⁶ mesenchymal stem cells comprise at most 1 mg mRNA construct at the time of administration.

24. The method according to claim 1, wherein the MSCs are administered at most 48 hours after transfection.

25. The method according to claim 1, wherein the subject is treated for pain.

26. A kit of parts of use in treatment of arthritis and/or pain, said kit of parts comprising:
a. Mesenchymal stem cells (MSCs) comprising an mRNA construct encoding TGFβ3 of SEQ ID NO: 29 or a functional homologue of said TGFβ3 sharing at least 70% sequence identity therewith;
b. MSCs comprising mRNA construct encoding
i. IL-10 of SEQ ID NO: 5 or a functional homologue of said IL-10 sharing at least 70% sequence identity therewith; or
ii. WNT3a of SEQ ID NO: 35 or a functional homologue of said WNT3a sharing at least 70% sequence identity therewith; or
iii. IGF1 of SEQ ID NO:23 or a functional homologue of said IGF1 sharing at least 70% sequence identity therewith; or
iv. BMP6 of SEQ ID NO: 17 or a functional homologue of said BMP6 sharing at least 70% sequence identity therewith; or
v. IL1RN of SEQ ID NO: 11 or a functional homologue of said IL1RN sharing at least 70% sequence identity therewith; and
c. Instructions for use in a subject comprising administering the MSC of a) at least 1 day after the MSCs of b).

27. A kit of parts of use in treatment of arthritis and/or pain, said kit of parts comprising:
a. Mesenchymal stem cells (MSCs) comprising an mRNA construct encoding TGFβ3 of SEQ ID NO: 29 or a functional homologue of said TGFβ3 sharing at least 70% sequence identity therewith;
b. MSCs comprising mRNA construct encoding
vi. IL-10 of SEQ ID NO: 5 or a functional homologue of said IL-10 sharing at least 70% sequence identity therewith; or
vii. WNT3a of SEQ ID NO: 35 or a functional homologue of said WNT3a sharing at least 70% sequence identity therewith; or
viii. IGF1 of SEQ ID NO:23 or a functional homologue of said IGF1 sharing at least 70% sequence identity therewith; or
ix. BMP6 of SEQ ID NO: 17 or a functional homologue of said BMP6 sharing at least 70% sequence identity therewith; or
x. IL1RN of SEQ ID NO: 11 or a functional homologue of said IL 1RN sharing at least 70% sequence identity therewith; and
c. Instructions for use in a subject comprising administering the MSC of a) at least 1 day before the MSCs of b).

28. A method of generating mesenchymal stem cells (MSCs), wherein the method comprises:
a. Providing MSCs; and
b. Transfecting the MSCs with an mRNA construct encoding TGFβ3 of SEQ ID NO: 29 or a functional homologue of said TGFβ3 sharing at least 70% sequence identity therewith.

29. The method according to claim 29, wherein the MSCs are generated by
a. culturing human pluripotent stem cells in a culture media comprising a WNT pathway agonist and a BET pathway antagonist for at least two days to generate induced cells; and
b. culturing the induced cells from step (a) in a culture media comprising a PDGF pathway agonist, an IGF1 pathway agonist and an FGF-beta pathway agonist for at least ten days.

30. The method according to claim 29, wherein the mRNA construct is transfected into the MSCs by a non-endosomal pathway of delivery.

31. The method according to claim 29, wherein the MSCs comprise a substantially single stranded composition of a mRNA construct transfected into the cell by a non-endosomal pathway of delivery, the mRNA construct comprising a coding sequence, wherein all nucleosides within the mRNA construct are chemically unmodified.

32. The method according to claim 29, wherein the codons of the coding sequence have been selected to reduce the uridine content by a method, wherein the method comprises following steps:
a. Codon optimization for expression in humans; and
b. Reduction of uridine content by selecting uridine low or uridine free codons.

33. The method according to claim 29, wherein the mRNA construct comprises at least one modified nucleotide base.

34. A method of expressing TGFβ3, the method comprising introducing an mRNA construct comprising mRNA having at least 70% sequence identity to SEQ ID NO: 25 into mesenchymal stem cells (MSCs) by a non-endosomal pathway of delivery.

35. An engineered mesenchymal stem cells (MSC) comprising an mRNA construct comprising mRNA having at least 70% sequence identity to SEQ ID NO: 25.

36. The method according to claim 1, wherein one MSC comprises at least 1×10⁴ copies of the mRNA construct at the time of administration.
